(19) United States
(12) Patent Application Publication
(10) Pub. No.: US 2024/0238323 A1
(43) Pub. Date: Jul. 18, 2024

(54) MODIFIED NUCLEOSIDES AND NUCLEOTIDES ANALOGS AS ANTIVIRAL AGENTS FOR CORONA AND OTHER VIRUSES

(71) Applicant: Emory University, Atlanta, GA (US)

(72) Inventors: Raymond Schinazi, Miami, FL (US); Franck Amblard, Atlanta, GA (US); HongWang Zhang, Atlanta, GA (US); Keivan Zandi, Poway, CA (US)

(21) Appl. No.: 18/286,163

(22) PCT Filed: Apr. 11, 2022
(86) PCT No.: PCT/US2022/024289

§ 371 Date: Oct. 9, 2023

Related U.S. Application Data
(60) Provisional application No. 63/173,354, filed on Apr. 9, 2021.
(60) Provisional application No. 63/175,673, filed on Apr. 16, 2021.
(60) Provisional application No. 63/210,246, filed on Jun. 14, 2021.
(60) Provisional application No. 63/288,163, filed on Dec. 10, 2021.
(60) Provisional application No. 63/298,836, filed on Jan. 12, 2022.

(51) Int. Cl. A61K 31/7072; A61K 31/52; A61K 31/675; A61K 31/706; A61K 31/7064; A61K 31/7068; A61K 45/06; A61P 31/14

(52) U.S. Cl. CPC A61K 31/7072; A61K 31/52; A61K 31/675; A61K 31/706; A61K 31/7064; A61K 31/7068; A61K 45/06; A61P 31/14

(57) ABSTRACT

Compounds, compositions and methods for preventing, treating or curing a coronavirus infection in human subjects or other animal hosts. In one embodiment, the compounds can be used to treat an infection with a severe acute respiratory syndrome virus, such as human coronavirus 229E, SARS, MERS, SARS-CoV-1 (OC43), and SARS-CoV-2. In another embodiment, the methods are used to treat a patient infected with a Flavivirus, Picornavus, Togavirus, or Bunyavirus.

### FIELD

The present disclosure is directed to compounds, methods and compositions for treating or preventing coronavirus infections. More specifically, the disclosure describes certain nucleoside and nucleotide analogs, pharmaceutically acceptable salts, or other derivatives thereof, and the use thereof in the treatment of coronaviruses, especially SARS-CoV-2.

### BACKGROUND

Coronaviruses are a species of virus belonging to the subfamily Coronavirinae in the family Coronaviridae, and are enveloped viruses with a positive-sense single-stranded RNA genome and with a nucleocapsid of helical symmetry.

Coronaviruses primarily infect the upper respiratory and gastrointestinal tract of mammals and birds, though several known strains infect humans as well. Coronaviruses are believed to cause a significant percentage of all common colds in human adults and children.

Coronaviruses cause colds in humans, primarily in the winter and early spring seasons. Coronaviruses can also cause pneumonia, either direct viral pneumonia or a secondary bacterial pneumonia, bronchitis, either direct viral bronchitis or a secondary bacterial bronchitis, and severe acute respiratory syndrome (SARS).

Coronaviruses also cause a range of diseases in farm animals and domesticated pets, some of which can be serious and are a threat to the farming industry. In chickens, the infectious bronchitis virus (IBV), a coronavirus, targets not only the respiratory tract but also the uro-genital tract. The virus can spread to different organs throughout the chicken.

Economically significant coronaviruses of farm animals include porcine coronavirus (transmissible gastroenteritis coronavirus, TGE) and bovine coronavirus, which both result in diarrhea in young animals. Feline Coronavirus: two forms, Feline enteric coronavirus is a pathogen of minor clinical significance, but spontaneous mutation of this virus can result in feline infectious peritonitis (FIP), a disease associated with high mortality. There are two types of canine coronavirus (CCoV), one that causes mild gastrointestinal disease and one that has been found to cause respiratory disease. Mouse hepatitis virus (MHV) is a coronavirus that causes an epidemic murine illness with high mortality, especially among colonies of laboratory mice.

Some strains of MHV cause a progressive demyelinating encephalitis in mice which has been used as a murine model for multiple sclerosis.

More recently a coronavirus pandemic has caused a dual threat to the health and the economy of the U.S. and the world. COVID-19 was first identified in December 2019 in Wuhan, Hubei province, China, resulting in the ongoing 2019-2020 pandemic. COVID-19 is caused by the severe acute respiratory syndrome coronavirus 2 (SARS-CoV-2). Common symptoms of the disease include fever (88%), dry cough (68%), shortness of breath (19%), and loss of smell (15 to 30%). Complications may include pneumonia, viral sepsis, acute respiratory distress syndrome, diarrhea, renal disease, cardiac issues and encephalitis. As of June, 2020, the total number of infected worldwide stood at over 4 million and at least 102,753 had died, and, according to the Johns Hopkins University Coronavirus Resource Center, almost two million people had tested positive for coronavirus in the U.S. and over one hundred thousand people had died of the disease. Local transmission of the disease has been recorded in over 200 countries. Risk factors include travel and viral exposure, and prevention is assisted by social distancing and quarantine.

Current treatments for these infections are mainly supportive, minimizing the symptoms rather than treating the underlying viral infection. For example, patients may be treated with analgesics to relieve pain, and patients with enteroviral carditis can be treated for complications such as arrhythmias, pericardial effusion, and cardiac failure.

It would be advantageous to provide new antiviral agents, compositions including these agents, and methods of treatment using these agents to treat coronaviruses. The present disclosure provides such agents, compositions and methods.

### SUMMARY

The present disclosure relates to compounds, methods and compositions for treating or preventing coronaviruses and/or other viral infections in a host. The methods involve administering a therapeutically or prophylactically-effective amount of at least one compound described herein to treat or prevent an infection by, or an amount sufficient to reduce the biological activity of, coronaviruses or other viral infections including, but not limited to, SARS-CoV-2, MERS, SARS, and OC-43. In other embodiments, the compounds described herein can be used for treating or preventing infections by Flaviviruses, Picornaviridae, Togavirodae and Bunyaviridae.

In one embodiment, methods of using potent, selective antiviral agents to target coronaviruses and other viral infections and thus help eliminate and/or treat infection in patients infected by these viruses are disclosed.

In one aspect of this embodiment, the compounds used include one or more of the specific nucleoside inhibitors described herein.

In another embodiment, pharmaceutical compositions including one or more of the compounds described herein are disclosed, which in one embodiment comprises a combination of a cytidine and a uridine analog, in combination with a pharmaceutically acceptable carrier or excipient. These compositions can be used to treat a host infected with a coronavirus or other viral infections, to prevent one of these infections, and/or to reduce the biological activity of one of these viruses. The compositions can include a combination of one or more of the compounds described herein, optionally with other antiviral compounds or biological agents, including anti-SARS-CoV2 compounds and biological agents, fusion inhibitors, entry inhibitors, protease inhibitors, polymerase inhibitors, antiviral nucleosides, such as remdesivir, GS-441524, N⁴-hydroxycytidine, and other compounds disclosed in U.S. Pat. No. 9,809,616, and their prodrugs, viral entry inhibitors, viral maturation inhibitors, JAK inhibitors, angiotensin-converting enzyme 2 (ACE2) inhibitors, SARS-CoV-specific human monoclonal antibodies, including CR3022, NS5A inhibitors, such as daclastavir, and agents of distinct or unknown mechanism.

In yet another embodiment, the present disclosure relates to processes for preparing the specific nucleoside compounds described herein.

In some embodiments, the compounds described herein are deuterated at one or more positions. Where the compounds are nucleosides, deuteration can be present in one or more positions on the sugar moiety of the compounds, the base portion of the compounds, and/or the prodrug portion of the compounds, at any position.

In some embodiments, ester prodrugs were prepared to allow more drug, when given orally, to reach the plasma and not be trapped in the gut as a triphosphate.

In another embodiment, ester prodrugs were prepared to improve the oral bioavailability of drugs.

The present disclosure will be better understood with reference to the following Detailed Description.

### BRIEF DESCRIPTION OF THE DRAWINGS

FIG. 1 shows the structure of the COVID-19 virus nsp12-nsp7-nsp8 complex, including the domain organization of COVID-19 virus nsp12.

FIG. 2 is a schematic illustration of the structure of the N-terminal NiRAN domain and hairpin of RdRp. The interacting residues in the palm and fingers subdomain of the RdRp domain and the NiRAN domain are identified by the labels.

FIG. 3 is a schematic illustration showing one embodiment of how an inhibitor triphosphate can interfere with RNA synthesis.

FIG. 4 is a photograph showing the degree of polymerase inhibition when Remdesivir, or a specific nucleotide inhibitor as described herein, is added, in a dose-dependent manner (1, 10, 100, 250, or 500 μM) to a mixture including an RdRp complex and nucleoside triphosphates, and one of water (control), Remdesivir, or an inhibitor compound is added.

### DETAILED DESCRIPTION

The compounds described herein show inhibitory activity against Coronaviridae in cell-based assays. Therefore, the compounds can be used to treat or prevent a Coronaviridae infection in a host, or reduce the biological activity of the virus. The host can be a mammal, and in particular, a human, infected with Coronaviridae virus. The compounds are also effective against Flaviviridae, Picornaviridae, Togavirodae and Bunyaviridae viruses. The methods involve administering an effective amount of one or more of the compounds described herein.

Pharmaceutical formulations including one or more compounds described herein, in combination with a pharmaceutically acceptable carrier or excipient, are also disclosed. In one embodiment, the formulations include at least one compound described herein and at least one further therapeutic agent.

The present disclosure will be better understood with reference to the following definitions:

### I. Definitions

The term “independently” is used herein to indicate that the variable, which is independently applied, varies independently from application to application. Thus, in a compound such as R″XYR″, wherein R″ is “independently carbon or nitrogen,” both R″ can be carbon, both R″ can be nitrogen, or one R″ can be carbon and the other R″ nitrogen.

As used herein, the term “enantiomerically pure” refers to a compound composition that comprises at least approximately 95%, and, preferably, approximately 97%, 98%, 99% or 100% of a single enantiomer of that compound.

As used herein, the term “substantially free of” or “substantially in the absence of” refers to a compound composition that includes at least 85 to 90% by weight, preferably 95% to 98% by weight, and, even more preferably, 99% to 100% by weight, of the designated enantiomer of that compound. In a preferred embodiment, the compounds described herein are substantially free of enantiomers.

Similarly, the term “isolated” refers to a compound composition that includes at least 85 to 90% by weight, preferably 95% to 98% by weight, and, even more preferably, 99% to 100% by weight, of the compound, the remainder comprising other chemical species or enantiomers.

The term “alkyl,” as used herein, unless otherwise specified, refers to a saturated straight, branched, or cyclic, primary, secondary, or tertiary hydrocarbons, including both substituted and unsubstituted alkyl groups. The alkyl group can be optionally substituted with any moiety that does not otherwise interfere with the reaction or that provides an improvement in the process, including but not limited to but limited to halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference. Specifically included are CF₃ and CH₂CF₃.

In the text, whenever the term C(alkyl range) is used, the term independently includes each member of that class as if specifically and separately set out. The term “alkyl” includes C₁₋₂₂ alkyl moieties, and the term “lower alkyl” includes C₁₋₆ alkyl moieties. It is understood to those of ordinary skill in the art that the relevant alkyl radical is named by replacing the suffix “-ane” with the suffix “-yl”.

As used herein, a “bridged alkyl” refers to a bicyclo- or tricyclo alkane, for example, a 2:1:1 bicyclohexane.

As used herein, a “spiro alkyl” refers to two rings that are attached at a single (quaternary) carbon atom.

The term “alkenyl” refers to an unsaturated, hydrocarbon radical, linear or branched, in so much as it contains one or more double bonds. The alkenyl group disclosed herein can be optionally substituted with any moiety that does not adversely affect the reaction process, including but not limited to but not limited to those described for substituents on alkyl moieties. Non-limiting examples of alkenyl groups include ethylene, methylethylene, isopropylidene, 1,2-ethane-diyl, 1,1-ethane-diyl, 1,3-propane-diyl, 1,2-propane-diyl, 1,3-butane-diyl, and 1,4-butane-diyl.

The term “alkynyl” refers to an unsaturated, acyclic hydrocarbon radical, linear or branched, in so much as it contains one or more triple bonds. The alkynyl group can be optionally substituted with any moiety that does not adversely affect the reaction process, including but not limited to those described above for alkyl moeities. Non-limiting examples of suitable alkynyl groups include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, and hexyn-3-yl, 3,3-dimethylbutyn-1-yl radicals.

The term “alkylamino” or “arylamino” refers to an amino group that has one or two alkyl or aryl substituents, respectively.

The term “fatty alcohol” as used herein refers to straight-chain primary alcohols with between 4 and 26 carbons in the chain, preferably between 8 and 26 carbons in the chain, and most preferably, between 10 and 22 carbons in the chain. The precise chain length varies with the source. Representative fatty alcohols include lauryl, stearyl, and oleyl alcohols. They are colourless oily liquids (for smaller carbon numbers) or waxy solids, although impure samples may appear yellow. Fatty alcohols usually have an even number of carbon atoms and a single alcohol group (—OH) attached to the terminal carbon. Some are unsaturated and some are branched. They are widely used in industry. As with fatty acids, they are often referred to generically by the number of carbon atoms in the molecule, such as “a C12 alcohol”, that is an alcohol having 12 carbons, for example dodecanol.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis, and are described, for example, in Greene et al., Protective Groups in Organic Synthesis, supra.

The term “aryl”, alone or in combination, means a carbocyclic aromatic system containing one, two or three rings wherein such rings can be attached together in a pendent manner or can be fused. Non-limiting examples of aryl include phenyl, biphenyl, or naphthyl, or other aromatic groups that remain after the removal of a hydrogen from an aromatic ring. The term aryl includes both substituted and unsubstituted moieties. The aryl group can be optionally substituted with any moiety that does not adversely affect the process, including but not limited to but not limited to those described above for alkyl moieties. Non-limiting examples of substituted aryl include heteroarylamino, N-aryl-N-alkylamino, N-heteroarylamino-N-alkylamino, heteroaralkoxy, arylamino, aralkylamino, arylthio, monoarylamidosulfonyl, arylsulfonamido, diarylamidosulfonyl, monoaryl amidosulfonyl, arylsulfinyl, arylsulfonyl, heteroarylthio, heteroarylsulfinyl, heteroarylsulfonyl, aroyl, heteroaroyl, aralkanoyl, heteroaralkanoyl, hydroxyaralkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, and heteroarylalkenyl, carboaralkoxy.

The terms “alkaryl” or “alkylaryl” refer to an alkyl group with an aryl substituent. The terms “aralkyl” or “arylalkyl” refer to an aryl group with an alkyl substituent.

The term “halo,” as used herein, includes chloro, bromo, iodo and fluoro.

The term “acyl” refers to a carboxylic acid ester in which the non-carbonyl moiety of the ester group is selected from the group consisting of straight, branched, or cyclic alkyl or lower alkyl, alkoxyalkyl, including, but not limited to methoxymethyl, aralkyl, including, but not limited to, benzyl, aryloxyalkyl, such as phenoxymethyl, aryl, including, but not limited to, phenyl, optionally substituted with halogen (F, Cl, Br, or I), alkyl (including but not limited to C₁, C₂, C₃, and C₄) or alkoxy (including but not limited to C₁, C₂, C₃, and C₄), sulfonate esters such as alkyl or aralkyl sulphonyl including but not limited to methanesulfonyl, the mono, di or triphosphate ester, trityl or monomethoxytrityl, substituted benzyl, trialkylsilyl (e.g., dimethyl-t-butylsilyl) or diphenylmethylsilyl. Aryl groups in the esters optimally comprise a phenyl group. The term “lower acyl” refers to an acyl group in which the non-carbonyl moiety is lower alkyl.

The terms “alkoxy” and “alkoxyalkyl” embrace linear or branched oxy-containing radicals having alkyl moieties, such as methoxy radical. The term “alkoxyalkyl” also embraces alkyl radicals having one or more alkoxy radicals attached to the alkyl radical, that is, to form monoalkoxyalkyl and dialkoxyalkyl radicals. The “alkoxy” radicals can be further substituted with one or more halo atoms, such as fluoro, chloro or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy.

The term “alkylamino” denotes “monoalkylamino” and “dialkylamino” containing one or two alkyl radicals, respectively, attached to an amino radical. The terms arylamino denotes “monoarylamino” and “diarylamino” containing one or two aryl radicals, respectively, attached to an amino radical. The term “aralkylamino”, embraces aralkyl radicals attached to an amino radical. The term aralkylamino denotes “monoaralkylamino” and “diaralkylamino” containing one or two aralkyl radicals, respectively, attached to an amino radical. The term aralkylamino further denotes “monoaralkyl monoalkylamino” containing one aralkyl radical and one alkyl radical attached to an amino radical.

The term “heteroatom,” as used herein, refers to oxygen, sulfur, nitrogen and phosphorus.

The terms “heteroaryl” or “heteroaromatic,” as used herein, refer to an aromatic that includes at least one sulfur, oxygen, nitrogen or phosphorus in the aromatic ring.

The term “heterocyclic,” “heterocyclyl,” and cycloheteroalkyl refer to a nonaromatic cyclic group wherein there is at least one heteroatom, such as oxygen, sulfur, nitrogen, or phosphorus in the ring.

Nonlimiting examples of heteroaryl and heterocyclic groups include furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, pyrrole, isopyrrole, pyrazole, imidazole, 1,2,3-triazole, 1,2,4-triazole, oxazole, isoxazole, thiazole, isothiazole, pyrimidine or pyridazine, and pteridinyl, aziridines, thiazole, isothiazole, 1,2,3-oxadiazole, thiazine, pyridine, pyrazine, piperazine, pyrrolidine, oxaziranes, phenazine, phenothiazine, morpholinyl, pyrazolyl, pyridazinyl, pyrazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, adenine, N⁶-alkylpurines, N⁶-benzylpurine, N⁶-halopurine, N⁶-vinypurine, N⁶-acetylenic purine, N⁶-acyl purine, N⁶-hydroxyalkyl purine, N⁶-thioalkyl purine, thymine, cytosine, 6-azapyrimidine, 2-mercaptopyrmidine, uracil, N⁵-alkylpyrimidines, N⁵-benzylpyrimidines, N⁵-halopyrimidines, N⁵-vinylpyrimidine, N⁵-acetylenic pyrimidine, N⁵-acyl pyrimidine, N⁵-hydroxyalkyl purine, and N⁶-thioalkyl purine, and isoxazolyl. The heteroaromatic group can be optionally substituted as described above for aryl. The heterocyclic or heteroaromatic group can be optionally substituted with one or more substituents selected from the group consisting of halogen, haloalkyl, alkyl, alkoxy, hydroxy, carboxyl derivatives, amido, amino, alkylamino, and dialkylamino. The heteroaromatic can be partially or totally hydrogenated as desired. As a nonlimiting example, dihydropyridine can be used in place of pyridine. Functional oxygen and nitrogen groups on the heterocyclic or heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-toluenelsulfonyl. The heterocyclic or heteroaromatic group can be substituted with any moiety that does not adversely affect the reaction, including but not limited to but not limited to those described above for aryl.

The term “host,” as used herein, refers to a unicellular or multicellular organism in which the virus can replicate, including but not limited to cell lines and animals, and, preferably, humans. Alternatively, the host can be carrying a part of the viral genome, whose replication or function can be altered by the compounds described herein. The term host specifically refers to infected cells, cells transfected with all or part of the viral genome and animals, in particular, primates (including but not limited to chimpanzees) and humans. In most animal applications described herein, the host is a human being. Veterinary applications, in certain indications, however, are clearly contemplated (such as for use in treating chimpanzees).

The term nucleoside also includes ribonucleosides, and representative ribonucleosides are disclosed, for example, in the Journal of Medicinal Chemistry, 43(23), 4516-4525 (2000), Antimicrobial Agents and Chemotherapy, 45(5), 1539-1546 (2001), and PCT WO 2000069876.

The term “peptide” refers to a natural or synthetic compound containing two to one hundred amino acids linked by the carboxyl group of one amino acid to the amino group of another.

The term “pharmaceutically acceptable salt or prodrug” is used throughout the specification to describe any pharmaceutically acceptable form (such as an ester) compound which, upon administration to a patient, provides the compound. Pharmaceutically-acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art.

Pharmaceutically acceptable prodrugs refer to a compound that is metabolized, for example hydrolyzed or oxidized, in the host to form the compound described herein. Typical examples of prodrugs include compounds that have biologically labile protecting groups on functional moieties of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. The prodrug forms of the compounds described herein can possess antiviral activity, can be metabolized to form a compound that exhibits such activity, or both.

### II. Active Compounds

The nucleoside compounds described herein are of one of the following formulas. In one embodiment, the compounds are compounds of Formula (A) or Formula (A1):

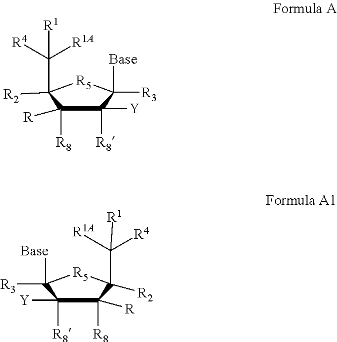

or a pharmaceutically acceptable salt or prodrug thereof, wherein:
Y and R are, independently, selected from the group consisting of H, OH, halo, an optionally substituted O-linked amino acid, substituted or unsubstituted C₁₋₆ alkyl, C₁₋₆ haloalkyl, C₁₋₆ alkoxy, substituted or unsubstituted C₂₋₆ alkenyl, substituted or unsubstituted C₂₋₆ alkynyl, substituted or unsubstituted C₃₋₆ cycloalkyl, cyano, cyanoalkyl, azido, azidoalkyl, OR′, SR′, wherein each R′ is independently a —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, C₁₋₆ alkyl, C₁₋₆ haloalkyl, C₁₋₆ alkoxy, C₂₋₆ alkenyl, C₂₋₆ alkynyl, and C₃₋₆ cycloalkyl, wherein the groups can be substituted with one or more substituents selected from the group consisting of halogen (fluoro, chloro, bromo or iodo), hydroxyl, amino, alkylamino, arylamino, alkoxy, nitro, and cyano,
R¹ is and R^{1A} are, independently, H, CH₃, CH₂F, CHF₂, or CF₃, wherein, when R¹ is Me, the carbon to which it is attached may be wholly or partially R or S or any mixture thereof, or R¹ and R^{1A} can combine to form a C₃₋₇ cycloalkyl ring;
R² is H, CN, N₃, F, CH₂-halogen, CH₂—N₃, O—CH₂—P—(OH)₃, substituted or unsubstituted C₁₋₈alkyl, substituted or unsubstituted C₂₋₈ alkenyl or substituted or unsubstituted C₂₋₈ alkynyl;
R³ is H, substituted or unsubstituted C₁₋₈alkyl, substituted or unsubstituted C₂₋₈ alkenyl, substituted or unsubstituted C₂₋₈ alkynyl, or N₃ when R⁵ is O, and R³ is selected from the group consisting of H, F, N₃, substituted or unsubstituted (C₁₋₈)alkyl, substituted or unsubstituted (C₂₋₈)alkenyl, substituted or unsubstituted (C₂₋₈)alkynyl, O—(C₁₋₈) alkyl and N₃ when R⁵ is Se, CH₂, CHF, CF₂, —C(CH₃)—, —C(cyclopropyl)-, C═CF₂ or C═CH₂,
R⁵ is O, CH₂, Se, CHF, CF₂, —C(CH₃)—, —C(cyclopropyl)-, C═CF₂ or C═CH₂,
R⁸ and R^{8′} are independently selected from the group consisting of H, OH, halo, an optionally substituted O-linked amino acid, substituted or unsubstituted C₁₋₆ alkyl, C₁₋₆ haloalkyl, C₁₋₆ alkoxy, substituted or unsubstituted C₂₋₆ alkenyl, substituted or unsubstituted C₂₋₆ alkynyl, substituted or unsubstituted C₃₋₆ cycloalkyl, cyano, cyanoalkyl, azido, azidoalkyl, OR′, SR′, wherein each R′ is independently a —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, C₁₋₆ alkyl, C₁₋₆ haloalkyl, C₁₋₆ alkoxy, C₂₋₆ alkenyl, C₂₋₆ alkynyl, C₃₋₆ cycloalkyl, wherein the groups can be substituted with one or more substituents selected from the group consisting of halogen (fluoro, chloro, bromo or iodo), hydroxyl, amino, alkylamino, arylamino, alkoxy, nitro, cyano,
R⁴ is OH, an optionally substituted O-linked amino acid, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, —O—C(O)O—C₃₋₆ cycloalkyl, OC₁₋₆ alkyl, OC₁₋₆ haloalkyl, OC₁₋₆ alkoxy, OC₂₋₆ alkenyl, OC₂₋₆ alkynyl, OC₃₋₆ cycloalkyl, O—P(O)R⁶R⁷, or a mono-, di-, or triphosphate, wherein, when chirality exists at the phosphorous center of R⁴, it may be wholly or partially Rₚ or Sₚ or any mixture thereof,
R⁶ and R⁷ are independently selected from the group consisting of:
(a) OR¹⁵ where R¹⁵ selected from the group consisting of H,

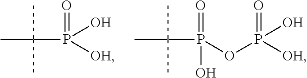

Li, Na, K, substituted or unsubstituted C₁₋₂₀alkyl, substituted or unsubstituted C₃₋₆cycloalkyl, C₁₋₄(alkyl)aryl, benzyl, C₁₋₆ haloalkyl, C₂₋₃(alkyl)OC₁₋₂₀alkyl, aryl, and heteroaryl, such as phenyl and pyridinyl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
where R¹⁶ is independently H, substituted or unsubstituted C₁₋₂₀ alkyl, the carbon chain derived from a fatty alcohol or C₁₋₂₀ alkyl substituted with a C₁₋₆ alkyl, C₁₋₆ alkoxy, di(C₁₋₆ alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆ alkyl, alkoxy, di(C₁₋₆ alkyl)-amino, fluoro, C₃₋₁₀ cycloalkyl, or cycloalkyl;
(b) the ester of a D- or L-amino acid

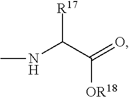

R¹⁷ and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl;

In one embodiment, Base is selected from the group consisting of:

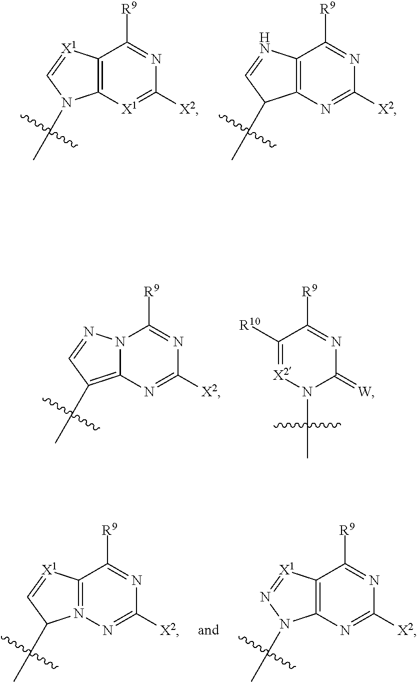

and in another embodiment, Base is:

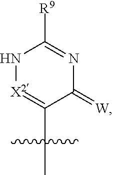

X¹ is CH, C—(C₁₋₆)alkyl, C—(C₂₋₆)alkenyl, C—(C₂₋₆)alkynyl, C—(C₃₋₇)cycloalkyl, C—(C₁₋₆) haloalkyl, C—(C₁₋₆)hydroxyalkyl, C—OR²², C—N(R²²)₂, C-halo, C—CN or N,
X^{1′} is CH, C—(C₁₋₆)alkyl, C—(C₂₋₆)alkenyl, C—(C₂₋₆)alkynyl, C-halo, C—CN or N
R⁹ and X² are independently H, OH, NH₂, halo (i.e., F, Cl, Br, or I), SH, NHOH, O(C₁₋₁₀)alkyl, O(C₂₋₁₀)alkene, O(C₂₋₁₀)alkyne, O(C₃₋₇)cycloalkyl, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, —O—C(O)O—C₃₋₆ cycloalkyl, S(C₁₋₁₀)alkyl, S(C₂₋₁₀)alkene, S(C₂₋₁₀)alkyne, S(C₃₋₇)cycloalkyl, an optionally unsaturated NH(C₁₋₁₀)alkyl, an optionally unsaturated N((C₁₋₁₀)alkyl)₂, NH(C₃₋₇)cycloalkyl, an optionally unsaturated NH(CO)(C₁₋₂₀)alkyl, an optionally unsaturated NH(CO)O(C₁₋₂₀)alkyl, NHOH, an optionally unsaturated NHO(CO)(C₁₋₂₀)alkyl, or an optionally unsaturated NHO(CO)NH(C₁₋₂₀)alkyl, (C₁₋₃)alkyl,
R^{9′} is OH, NH₂, SH, NHOH, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, or —O—C(O)O—C₃₋₆ cycloalkyl,
R¹⁰ is H or F,
X^{2′} is N or CH, and
W is O or S.

In one embodiment, R⁵ is O.

In another embodiment, R² is H or substituted or unsubstituted C₂₋₈ alkynyl.

In another embodiment, R³ is H.

In another embodiment, R³ is H or substituted or unsubstituted C₂₋₈ alkynyl.

In another embodiment, R² is CN or H.

In another embodiment, R¹ is and R^{1A} are H.

In another embodiment, R⁸ and R^{8′} are OH.

In another embodiment, R⁴ is OH or O—P(O)R⁶R⁷.

In another embodiment, Base is

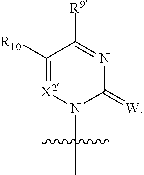

In one aspect of this embodiment, R⁹ is OH, NH₂, or NHOH

In another embodiment, Base is

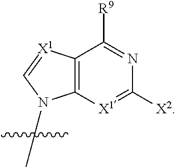

In one aspect of this embodiment, X² is NH₂, OH or SH.

These subfeatures can be present in any combination in any compound described herein.

In another embodiment, the compounds are compounds of Formula (B) or (B1):

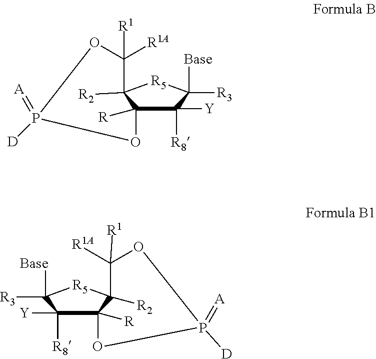

or a pharmaceutically acceptable salt or prodrug thereof, wherein:
Base, Y, R, R¹, R^{1A}, R², R³, R⁵, and R^{8′} are as defined in Formula A,
A is O or S, and
D is selected from the group consisting of:
(a) OR¹⁵ where R¹⁵ is selected from the group consisting of H, substituted or unsubstituted C₁₋₂₀alkyl, substituted or unsubstituted C₃₋₆cycloalkyl, C₁₋₄(alkyl)aryl, benzyl, C₁₋₆haloalkyl, C₂₋₃(alkyl)OC₁₋₂₀ alkyl, aryl, and heteroaryl, such as phenyl and pyridinyl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
(b) the ester of a D- or L-amino acid

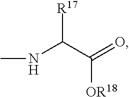

R¹⁷ and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl; and
(c)

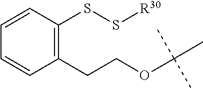

where R³⁰ is selected from the group consisting of substituted or unsubstituted C₁₋₂₀alkyl, substituted or unsubstituted C₃₋₆ cycloalkyl, substituted or unsubstituted (C₂₋₁₀)alkene, substituted or unsubstituted (C₂₋₁₀)alkyne, C₁₋₄(alkyl)aryl, aryl, heteroaryl, and C₁₋₆ haloalkyl.

In one embodiment, R⁵ is O.

In another embodiment, R² is H or substituted or unsubstituted C₂₋₈ alkynyl.

In another embodiment, R³ is H.

In another embodiment, R³ is H or substituted or unsubstituted C₂₋₈ alkynyl.

In another embodiment, R² is CN or H.

In another embodiment, R^{8′} is OH.

In another embodiment, Y is H.

In another embodiment, R¹ and R^{1A} are H.

In another embodiment, A is O.

These subfeatures can be present in any combination in any compound described herein.

In another embodiment, the compounds are compounds of Formula (C) or (C1):

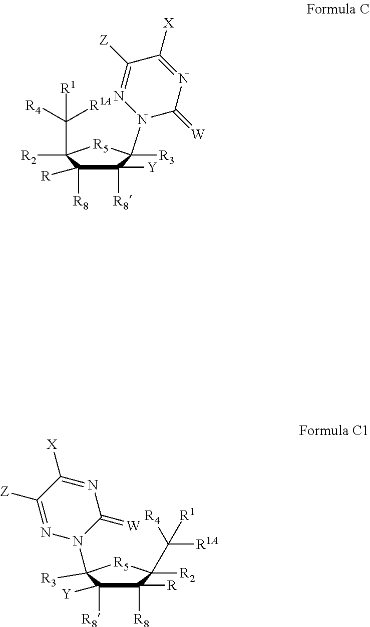

or a pharmaceutically acceptable salt or prodrug thereof, wherein:
R, R¹, R^{1A}, R², R³, R⁵, R⁸, R^{8′} and Y are as defined in Formula A,
X is OH, NH₂, SH, NHOH, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, or —O—C(O)O—C₃₋₆ cycloalkyl,
Z is H or F, and
W is O or S.

In one embodiment, R⁵ is O.

In another embodiment, R² is N₃ or substituted or unsubstituted C₂₋₈ alkynyl.

In another embodiment, R³ is H.

In another embodiment, R³ is N₃ or substituted or unsubstituted C₂₋₈ alkynyl.

In another embodiment, R² is CN or H.

In one embodiment, R⁸ and R^{8′} are OH

In one embodiment, Y is H.

In one embodiment, R is H.

In one embodiment, Z is H.

In one embodiment, X is OH, NH₂ or NHOH.

In one embodiment, W is O.

In one embodiment, R¹ and R^{1A} are H.

In one embodiment, R⁴ is OH or O—P(O)R⁶R⁷.

These subfeatures can be present in any combination in any compound described herein.

In another embodiment, the compounds are compounds of Formula (D) or (D1).

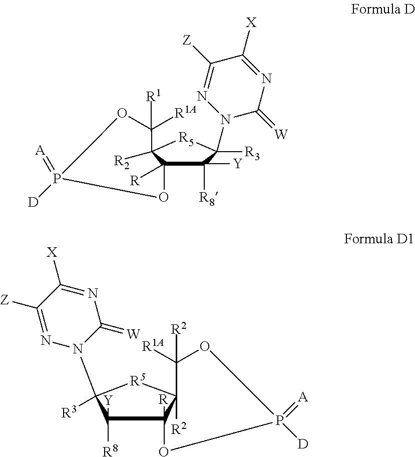

or a pharmaceutically acceptable salt or prodrug thereof, wherein R, R¹, R^{1A}, R², R³, R⁵, R^{8′} and Y are as defined in Formula A, and A and D are as defined in Formula C.

In one embodiment, R⁵ is O.

In another embodiment, R² is H or substituted or unsubstituted C₂₋₈ alkynyl.

In another embodiment, R³ is H.

In another embodiment, R³ is H or substituted or unsubstituted C₂₋₈ alkynyl.

In another embodiment, R² is CN or H.

In another embodiment, R^{8′} is OH.

In another embodiment, Y is H.

In another embodiment, R is H.

In another embodiment, Z is H.

In another embodiment, X is OH, NH₂ or NHOH.

In another embodiment, W is O.

In another embodiment, R¹ and R^{1A} are H.

In another embodiment, R⁴ is OH or O—P(O)R⁶R⁷.

These subfeatures can be present in any combination in any compound described herein.

In another embodiment, the compounds are compounds of Formula (E) or (E1):

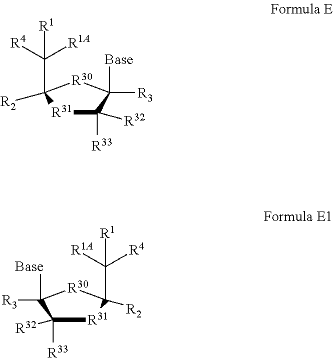

or a pharmaceutically acceptable salt or prodrug thereof, wherein:
Base, R¹, R^{1A}, R², R³, and R⁴ are as defined in Formula A,
R³⁰ is O or CH₂,
R³¹ is O or S when R³⁰ is O or CH₂,
R³² and R³³ are independently H, F, C₁-C₃ alkyl, C₂-C₃ alkene, or C₂-C₃ alkyne.

In one embodiment, R³⁰ is O.

In another embodiment, R³¹ is O.

In another embodiment, R³² and R³³ are, independently, H or F.

In another embodiment, R² is N₃ or substituted or unsubstituted C₂₋₈ alkynyl.

In another embodiment, R³ is N₃ or substituted or unsubstituted C₂₋₈ alkynyl.

In another embodiment, R² is CN.

In another embodiment, R¹ and R^{1A} are H.

In another embodiment, R⁴ is OH or O—P(O)R⁶R⁷.

In another embodiment, Base is

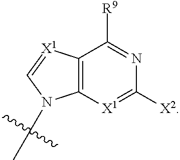

In another embodiment, X¹ is N.

These subfeatures can be present in any combination in any compound described herein.

In another embodiment, the compounds are compounds of Formula (F) or (F1):

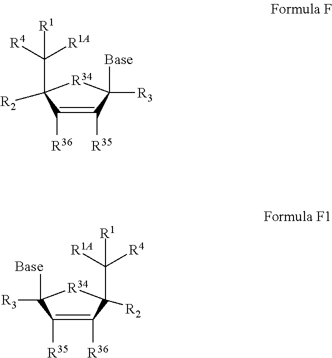

or a pharmaceutically acceptable salt or prodrug thereof, wherein:
Base, R¹, R^{1A}, R², R³, and R⁴ are as defined in Formula A,
R³⁴ is O or CH₂, and
R³⁵ and R³⁶ are independently H, F or CH₃.

In embodiment, R³⁵ and R³⁶ are H.

In one embodiment, R³⁴ is CH₂.

In one embodiment, R⁴ is OH or O—P(O)R⁶R⁷.

In one embodiment, R³ is H.

In one embodiment, R² is H or substituted or unsubstituted C₂₋₈ alkynyl.

In one embodiment, R² is CN or N₃.

In one embodiment, R³ is substituted or unsubstituted C₂₋₈ alkynyl.

In one embodiment, R¹ and R^{1A} are H.

These subfeatures can be present in any combination in any compound described herein.

In another embodiment, the compounds have one of the following formulas:

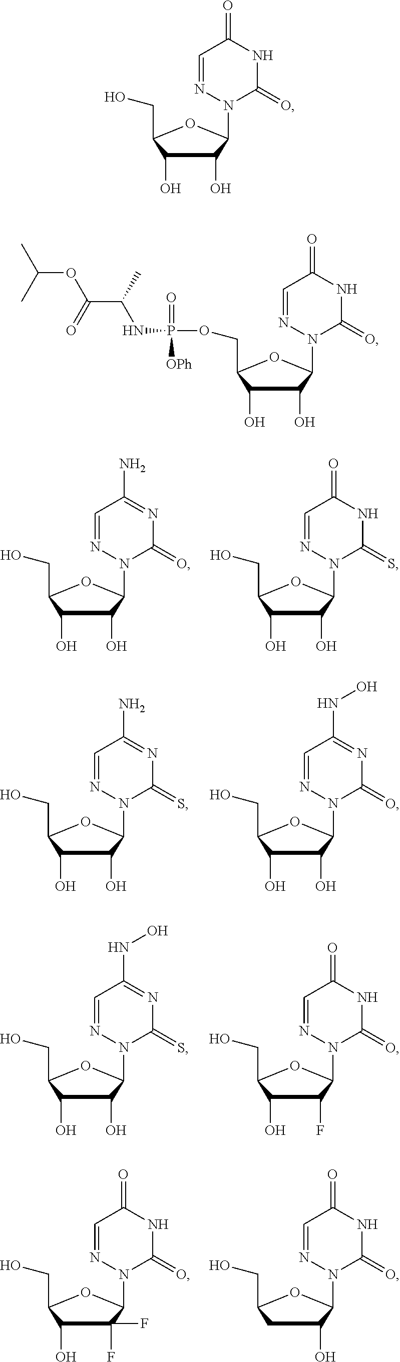

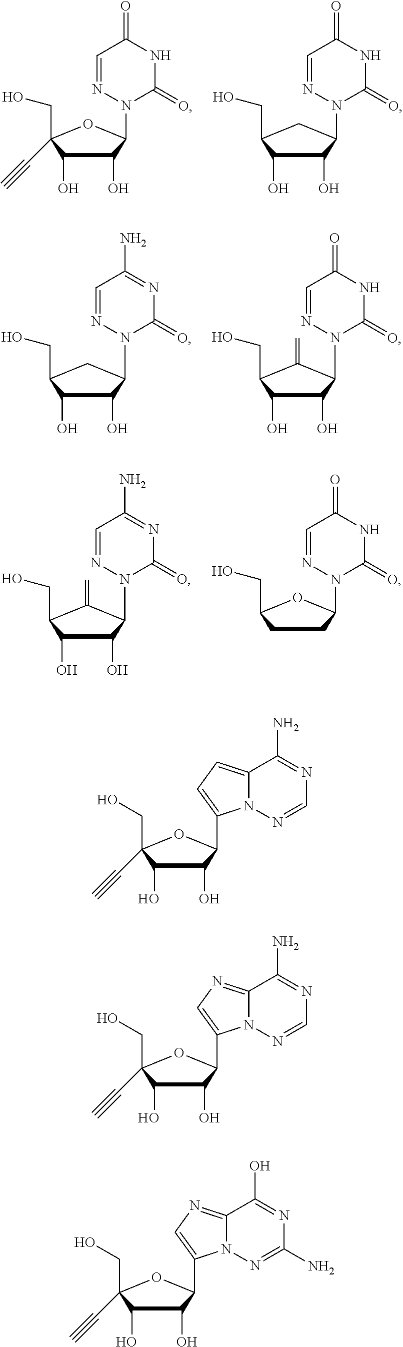

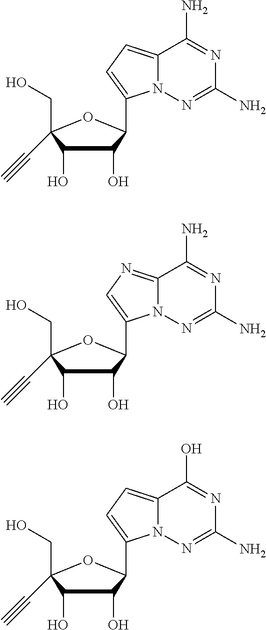

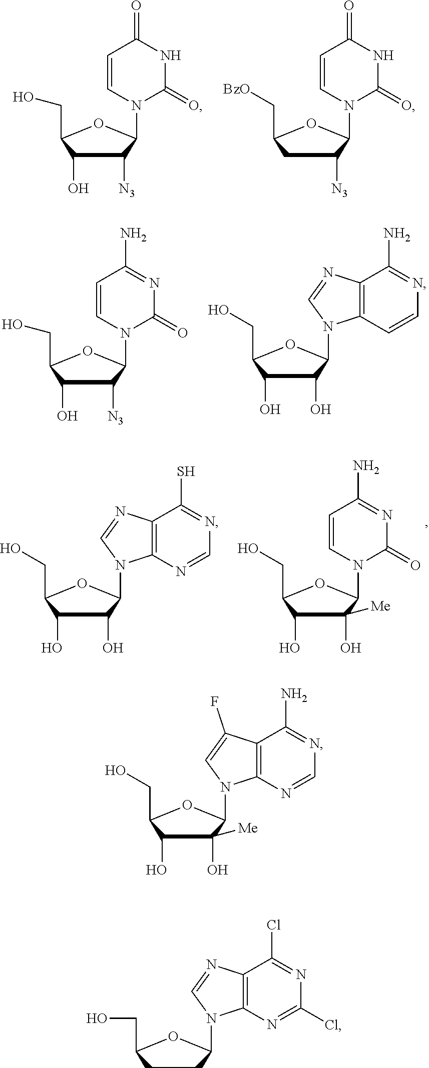

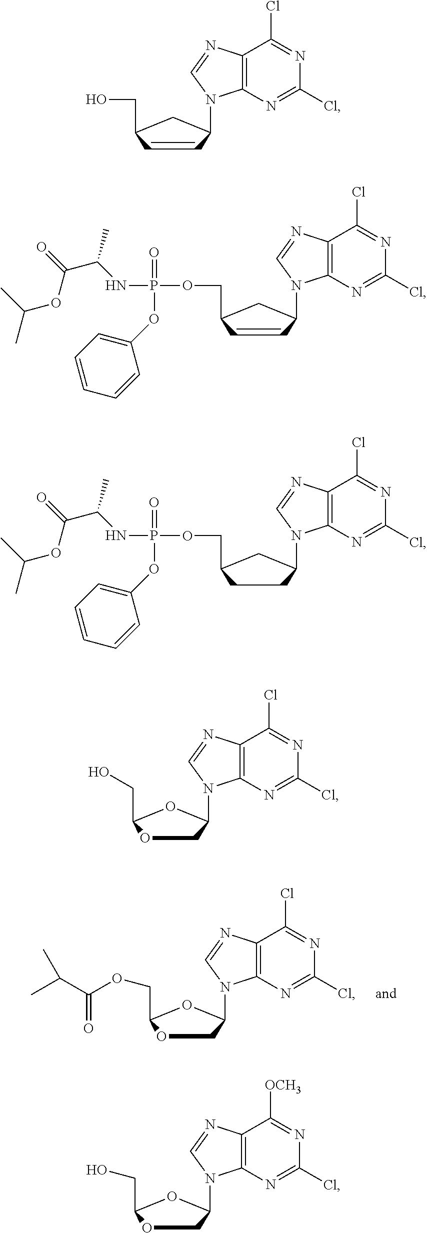

or a pharmaceutically acceptable salt or prodrug thereof.

In one embodiment, the compounds have one of the following formulas:

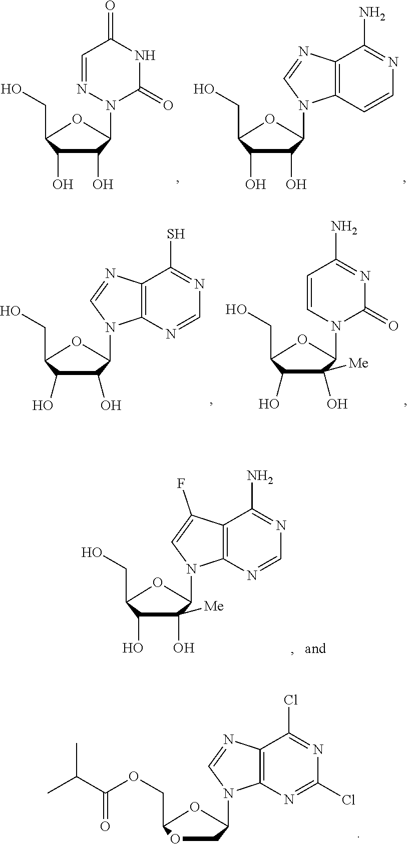

In another embodiment, the compounds have one of the following formulas:

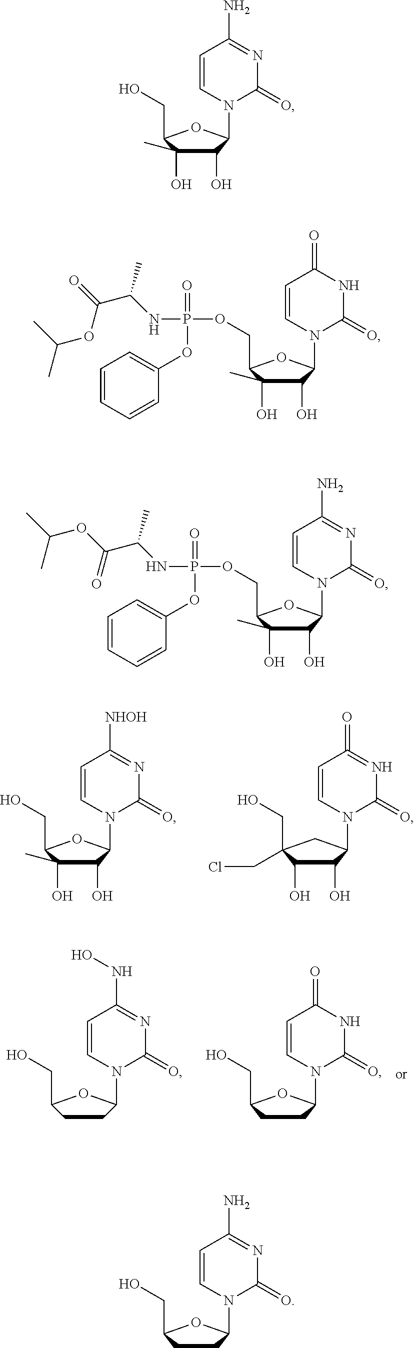

In any of these embodiments, the compounds can be present in the 3-D or 3-L configuration.

### III Stereoisomerism and Polymorphism

The compounds described herein can have asymmetric centers and occur as racemates, racemic mixtures, individual diastereomers or enantiomers, with all isomeric forms being included in the present disclosure. Compounds described herein having a chiral center can exist in and be isolated in optically active and racemic forms. Some compounds can exhibit polymorphism. The present disclosure encompasses racemic, optically-active, polymorphic, or stereoisomeric forms, or mixtures thereof, of a compound described herein, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution. One can either purify the respective compound, then derivatize the compound to form the compounds described herein, or purify the compound themselves.

Optically active forms of the compounds can be prepared using any method known in the art, including but not limited to by resolution of the racemic form by recrystallization techniques, by synthesis from optically-active starting materials, by chiral synthesis, or by chromatographic separation using a chiral stationary phase.

Examples of methods to obtain optically active materials include at least the following.
i) physical separation of crystals: a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization: a technique whereby the individual enantiomers are separately crystallized from a solution of the racemate, possible only if the latter is a conglomerate in the solid state;
iii) enzymatic resolutions: a technique whereby partial or complete separation of a racemate by virtue of differing rates of reaction for the enantiomers with an enzyme;
iv) enzymatic asymmetric synthesis: a synthetic technique whereby at least one step of the synthesis uses an enzymatic reaction to obtain an enantiomerically pure or enriched synthetic precursor of the desired enantiomer;
v) chemical asymmetric synthesis: a synthetic technique whereby the desired enantiomer is synthesized from an achiral precursor under conditions that produce asymmetry (i.e., chirality) in the product, which can be achieved using chiral catalysts or chiral auxiliaries;
vi) diastereomer separations: a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations: a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions: this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors: a synthetic technique whereby the desired enantiomer is obtained from non-chiral starting materials and where the stereochemical integrity is not or is only minimally compromised over the course of the synthesis;
x) chiral liquid chromatography: a technique whereby the enantiomers of a racemate are separated in a liquid mobile phase by virtue of their differing interactions with a stationary phase (including but not limited to via chiral HPLC). The stationary phase can be made of chiral material or the mobile phase can contain an additional chiral material to provoke the differing interactions;
xi) chiral gas chromatography: a technique whereby the racemate is volatilized and enantiomers are separated by virtue of their differing interactions in the gaseous mobile phase with a column containing a fixed non-racemic chiral adsorbent phase;
xii) extraction with chiral solvents: a technique whereby the enantiomers are separated by virtue of preferential dissolution of one enantiomer into a particular chiral solvent;
xiii) transport across chiral membranes: a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

Chiral chromatography, including but not limited to simulated moving bed chromatography, is used in one embodiment. A wide variety of chiral stationary phases are commercially available.

### IV. Salt or Prodrug Formulations

In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate and α-glycerophosphate. Suitable inorganic salts can also be formed, including but not limited to, sulfate, nitrate, bicarbonate and carbonate salts. For certain transdermal applications, it can be preferred to use fatty acid salts of the compounds described herein. The fatty acid salts can help penetrate the stratum corneum. Examples of suitable salts include salts of the compounds with stearic acid, oleic acid, lineoleic acid, palmitic acid, caprylic acid, and capric acid.

Pharmaceutically acceptable salts can be obtained using standard procedures well known in the art, for example by reacting a sufficiently basic compound such as an amine with a suitable acid, affording a physiologically acceptable anion. In those cases where a compound includes multiple amine groups, the salts can be formed with any number of the amine groups. Alkali metal (e.g., sodium, potassium or lithium) or alkaline earth metal (e.g., calcium) salts of carboxylic acids can also be made.

A prodrug is a pharmacological substance that is administered in an inactive (or significantly less active) form and subsequently metabolized in vivo to an active metabolite. Getting more drug to the desired target at a lower dose is often the rationale behind the use of a prodrug and is generally attributed to better absorption, distribution, metabolism, and/or excretion (ADME) properties. Prodrugs are usually designed to improve oral bioavailability, with poor absorption from the gastrointestinal tract usually being the limiting factor. Additionally, the use of a prodrug strategy can increase the selectivity of the drug for its intended target thus reducing the potential for off target effects.

### V. Methods of Treatment

In one embodiment, the compounds described herein can be used to prevent, treat or cure coronavirus infections, specifically including SARS-CoV2 infections, such as SARS-CoV-2, MERS, SARS, and OC-43. In other embodiments, the compounds described herein can be used to prevent, treat or cure infections by Flaviviruses, Picornaviridae, Togavirodae and Bunyaviridae.

The methods involve administering a therapeutically or prophylactically-effective amount of at least one compound as described herein to treat, cure or prevent an infection by, or an amount sufficient to reduce the biological activity of, a coronavirus infection, or an infection caused by a Flavivirus, Picornavus, Togavirus, or Bunyavirus, or other RNA virus.

In another embodiment, the compounds described herein can be used to inhibit a coronoviral, flaviviral, picornaviral, togaviral, or bunyaviral protease, or protease associated with another RNA virus, in a cell. The method includes contacting the cell with an effective amount of a compound described herein,

Hosts, including but not limited to humans infected with a coronavirus, flavivirus, picornavirus, togavirus, or bunyavirus, or other RNA virus, or a gene fragment thereof, can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, transdermally, subcutaneously, or topically, in liquid or solid form.

There are several species within the Coronavirus genus including, but not limited to, Middle East respiratory syndrome coronavirus (MERS-CoV), SARS coronavirus (SARS-CoV) and SARS-Cov2. In some embodiments, a compound described herein can ameliorate and/or treat a MERS-CoV infection, SARS-CoV infection, or SARS-Cov2 infection. An effective amount of a compound described herein can be administered to a subject infected with these viruses, and/or by contacting a cell infected with these viruses with an effective amount of a compound described herein. In some embodiments, a compound described herein can inhibit replication of these viruses. In some embodiments, a compound described herein can ameliorate one or more symptoms of these infections. Symptoms include, but are not limited to, extreme fatigue, malaise, headache, high fever (e.g., >100.4° F.), lethargy, confusion, rash, loss of appetite, myalgia, chills, diarrhea, dry cough, runny nose, sore throat, shortness of breath, breathing problems, gradual fall in blood-oxygen levels (such as, hypoxia) and pneumonia.

Some embodiments disclosed herein relate to a method of treating and/or ameliorating an infection caused by a Togaviridae virus that can include administering to a subject an effective amount of one or more compounds described herein, or a pharmaceutical composition that includes a compound described herein. Some embodiments described herein relate to using one or more compounds described herein in the manufacture of a medicament for ameliorating and/or treating an infection caused by a Togaviridae virus that can include administering to a subject an effective amount of one or more compounds described herein.

Some embodiments disclosed herein relate to methods of ameliorating and/or treating an infection caused by a Togaviridae virus that can include contacting a cell infected with the virus with an effective amount of one or more compounds described herein, or a pharmaceutical composition that includes one or more compounds described herein. Other embodiments described herein relate to using one or more compounds described herein in the manufacture of a medicament for ameliorating and/or treating an infection caused by a Togaviridae virus that can include contacting a cell infected with the virus with an effective amount of said compound(s).

In some embodiments, the Togaviridae virus can be an Alphavirus. One species of an Alphavirus is a Venezuelan equine encephalitis virus (VEEV). In some embodiments, a compound described herein can ameliorate and/or treat a VEEV infection. In other embodiments, one or more compounds described herein, can be manufactured into a medicament for ameliorating and/or treating an infection caused by a VEEV that can include contacting a cell infected with the virus with an effective amount of said compound(s). In still other embodiments, one or more compounds described herein, can be used for ameliorating and/or treating an infection caused by a VEEV that can include contacting a cell infected with the virus with an effective amount of said compound(s). In some embodiment, the VEEV can be an epizootic subtype. In some embodiment, the VEEV can be an enzootic subtype. As described herein, the Venezuelan equine encephalitis complex of viruses includes multiple subtypes that are further divided by antigenic variants. In some embodiments, a compound described herein can be effective against more than one subtype of a VEEV, such as 2, 3, 4, 5 or 6 subtypes. In some embodiments, a compound can be used to treat, ameliorate and/or prevent VEEV subtype I. In some embodiments, a compound described herein can be effective against more than one antigenic variants of a VEEV. In some embodiments, a compound can ameliorate one or more symptoms of a VEEV infection. Examples of symptoms manifested by a subject infected with VEEV include flu-like symptoms, such as high fever, headache, myalgia, fatigue, vomiting, nausea, diarrhea, and pharyngitis. Subjects with encephalitis show one or more of the following symptoms: somnolence, convulsions, confusion, photophobia, coma and bleeding of the brain, lung(s) and/or gastrointestinal tract. In some embodiments, the subject can be human. In other embodiments, the subject can be a horse.

Chikungunya (CHIKV) is another Alphavirus species. In some embodiments, a compound described herein can ameliorate and/or treat a CHIKV infection. In other embodiments, one or more compounds described herein can be manufactured into a medicament for ameliorating and/or treating an infection caused by a CHIKV that can include contacting a cell infected with the virus with an effective amount of said compound(s). In still other embodiments, one or more compounds described herein, can be used for ameliorating and/or treating an infection caused by a CHIKV that can include contacting a cell infected with the virus with an effective amount of said compound(s). In some embodiments, one or more symptoms of a CHIKV infection can be ameliorated by administering an effective amount of a compound to a subject infected with CHIKV and/or by contacting an CHIKV infected cell with an effective amount of a compound described herein. Clinical symptoms of a CHIKV infection include fever, rash (such as petechial and/or maculopapular rash), muscle pain, joint pain, fatigue, headache, nausea, vomiting, conjunctivitis, loss of taste, photophobia, insomnia, incapacitating joint pain and arthritis.

Other species of Alphaviruses include Barmah Forest virus, Mayaro virus (MAYV), O'nyong'nyong virus, Ross River virus (RRV), Semliki Forest virus, Sindbis virus (SINV), Una virus, Eastern equine encephalitis virus (EEE) and Western equine encephalomyelitis (WEE). In some embodiments, one or more compounds described herein, can be used for ameliorating and/or treating an infection caused by an Alphavirus that can include contacting a cell infected with the virus with an effective amount of one or more of said compound(s) and/or administering to a subject (such as, a subject infected with the virus) an effective amount of one or more of said compound(s), wherein the Alphavirus can be selected from Barmah Forest virus, Mayaro virus (MAYV), O'nyong'nyong virus, Ross River virus (RRV), Semliki Forest virus, Sindbis virus (SINV), Una virus, Eastern equine encephalitis virus (EEE) and Western equine encephalomyelitis (WEE).

Another genus of a Coronaviridae virus is a Rubivirus. Some embodiments disclosed herein relate to methods of ameliorating and/or treating an infection caused by a Rubivirus that can include contacting a cell infected with the virus with an effective amount of one or more compounds described herein, or a pharmaceutical composition that includes one or more compounds described herein. Other embodiments described herein relate to using one or more compounds described herein, in the manufacture of a medicament for ameliorating and/or treating an infection caused by a Rubivirus that can include contacting a cell infected with the virus with an effective amount of said compound(s). Still other embodiments described herein relate to one or more compounds described herein, that can be used for ameliorating and/or treating an infection caused by a Rubivirus by contacting a cell infected with the virus with an effective amount of said compound(s).

Some embodiments disclosed herein relate to a method of treating and/or ameliorating an infection caused by a Bunyaviridae virus that can include administering to a subject an effective amount of one or more compounds described herein, or a pharmaceutical composition that includes a compound described herein. Other embodiments disclosed herein relate to a method of treating and/or ameliorating an infection caused by a Bunyaviridae virus that can include administering to a subject identified as suffering from the viral infection an effective amount of one or more compounds described herein, or a pharmaceutical composition that includes a compound described herein.

Some embodiments disclosed herein relate to methods of ameliorating and/or treating an infection caused by a Bunyaviridae virus that can include contacting a cell infected with the virus with an effective amount of one or more compounds described herein, or a pharmaceutical composition that includes one or more compounds described herein. Other embodiments described herein relate to using one or more compounds described herein, in the manufacture of a medicament for ameliorating and/or treating an infection caused by a Bunyaviridae virus that can include contacting a cell infected with the virus with an effective amount of said compound(s). Still other embodiments described herein relate to one or more compounds described herein, that can be used for ameliorating and/or treating an infection caused by a Bunyaviridae virus by contacting a cell infected with the virus with an effective amount of said compound(s).

Some embodiments disclosed herein relate to methods of inhibiting replication of a Bunyaviridae virus that can include contacting a cell infected with the virus with an effective amount of one or more compounds described herein, or a pharmaceutical composition that includes one or more compounds described herein. Other embodiments described herein relate to using one or more compounds described herein, in the manufacture of a medicament for inhibiting replication of a Bunyaviridae virus that can include contacting a cell infected with the virus with an effective amount of said compound(s). Still other embodiments described herein relate to a compound described herein, that can be used for inhibiting replication of a Bunyaviridae virus by contacting a cell infected with the virus with an effective amount of said compound(s). In some embodiments, a compound described herein can inhibit a RNA dependent RNA polymerase of a Bunyaviridae virus, and thereby, inhibit the replication of RNA. In some embodiments, a polymerase of a Bunyaviridae virus can be inhibited by contacting a cell infected with the Bunyaviridae virus with a compound described herein.

In some embodiments, the Bunyaviridae virus can be a Bunyavirus. In other embodiments, the Bunyaviridae virus can be a Hantavirus. In still other embodiments, the Bunyaviridae virus can be a Nairovirus. In yet still other embodiments, the Bunyaviridae virus can be a Phlebovirus. In some embodiments, the Bunyaviridae virus can be an Orthobunyavirus. In other embodiments, the Bunyaviridae virus can be a Tospovirus.

A species of the Phlebovirus genus is Rift Valley Fever virus. In some embodiments, a compound described herein can ameliorate and/or treat a Rift Valley Fever virus infection. In other embodiments, one or more compounds described herein, can be manufactured into a medicament for ameliorating and/or treating an infection caused by a Rift Valley Fever virus that can include contacting a cell infected with the virus with an effective amount of said compound(s). In still other embodiments, one or more compounds described herein can be used for ameliorating and/or treating an infection caused by a Rift Valley Fever virus that can include contacting a cell infected with the virus with an effective amount of said compound(s). In some embodiments, a compound described herein can inhibit replication of Rift Valley Fever virus, wherein said compound is administering to a subject infected with Rift Valley Fever virus and/or wherein said compound contacts a cell infected with Rift Valley Fever.

In some embodiments, a compound described herein can ameliorate, treat, and/or inhibit replication of one or more of the ocular form, the meningoencephalitis form, or the hemorrhagic fever form of Rift Valley Fever virus. In some embodiments, one or more symptoms of a Rift Valley Fever virus infection can be ameliorated. Examples of symptoms of a Rift Valley Fever viral infection include headache, muscle pain, joint pain, neck stiffness, sensitivity to light, loss of appetite, vomiting, myalgia, fever, fatigue, back pain, dizziness, weight loss, ocular form symptoms (for example, retinal lesions, blurred vision, decreased vision and/or permanent loss of vision), meningoencephalitis form symptoms (such as, intense headache, loss of memory, hallucinations, confusion, disorientation, vertigo, convulsions, lethargy and coma) and hemorrhagic fever form symptoms (for example, jaundice, vomiting blood, passing blood in the feces, a purpuric rash, ecchymoses, bleeding from the nose and/or gums, menorrhagia and bleeding from a venepuncture site).

Another species of the Phlebovirus genus is thrombocytopenia syndrome virus. In some embodiments, a compound described herein can ameliorate, treat, and/or inhibit replication thrombocytopenia syndrome virus. In some embodiments, a compound can ameliorate and/or treat severe fever with thrombocytopenia syndrome (SFTS). In some embodiments, a compound described herein can ameliorate one or more symptoms of SFTS. Clinical symptoms of include the following: fever, vomiting, diarrhea, multiple organ failure, thrombocytopenia, leucopenia, and elevated liver enzyme levels.

Crimean-Congo hemorrhagic fever virus (CCHF) is a species within the Nairovirus genus. In some embodiments, a compound described herein can ameliorate, treat, and/or inhibit replication of Crimean-Congo hemorrhagic fever virus. Subjects infected with CCHF have one or more of the following symptoms: flu-like symptoms (such as high fever, headache, myalgia, fatigue, vomiting, nausea, diarrhea, and/or pharyngitis), hemorrhage, mood instability, agitation, mental confusion, throat petechiae, nosebleeds, bloody urine, vomiting, black stools, swollen and/or painful liver, disseminated intravascular coagulation, acute kidney failure, shock and acute respiratory distress syndrome. In some embodiments, a compound described herein can ameliorate one or more symptoms of CCHF.

California encephalitis virus is another virus of the Bunyaviridae family, and is a member of the Orthobunavirus genus. Symptoms of a California encephalitis virus infection include, but are not limited to fever, chills, nausea, vomiting, headache, abdominal pain, lethargy, focal neurologic findings, focal motor abnormalities, paralysis, drowsiness, lack of mental alertness and orientation and seizures. In some embodiments, a compound described herein can ameliorate, treat, and/or inhibit replication of California encephalitis virus. In some embodiments, a compound described herein can ameliorate one or more symptoms of a California encephalitis viral infection.

Viruses within the Hantavirus genus can cause hantavirus hemorrhagic fever with renal syndrome (HFRS) (caused by viruses such as Hantaan River virus, Dobrava-Belgrade virus, Saaremaa virus, Seoul virus, and Puumala virus) and hantavirus pulmonary syndrome (HPS). Viruses that can cause HPS include, but are not limited to, Black Creek Canal virus (BCCV), New York virus (NYV), Sin Nombre virus (SNV). In some embodiments, a compound described herein can ameliorate and/or treat HFRS or HPS. Clinical symptoms of HFRS include redness of cheeks and/or nose, fever, chills, sweaty palms, diarrhea, malaise, headaches, nausea, abdominal and back pain, respiratory problems, gastro-intestinal problems, tachycardia, hypoxemia, renal failure, proteinuria and diuresis. Clinical symptoms of HPS include flu-like symptoms (for example, cough, myalgia, headache, lethargy and shortness-of-breath that can deteriorate into acute respiratory failure). In some embodiments, a compound described herein can ameliorate one or more symptoms of IFRS or HPS.

Various indicators for determining the effectiveness of a method for treating and/or ameliorating a Coronaviridae, a Togaviridae, a Hepeviridae and/or a Bunyaviridae viral infection are known to those skilled in the art. Example of suitable indicators include, but are not limited to, a reduction in viral load, a reduction in viral replication, a reduction in time to seroconversion (virus undetectable in patient serum), a reduction of morbidity or mortality in clinical outcomes, and/or other indicator(s) of disease response. Further indicators include one or more overall quality of life health indicators, such as reduced illness duration, reduced illness severity, reduced time to return to normal health and normal activity, and reduced time to alleviation of one or more symptoms. In some embodiments, a compound described herein can result in the reduction, alleviation or positive indication of one or more of the aforementioned indicators compared to a subject who is untreated subject.

### VI. Combination or Alternation Therapy

In one embodiment, the compounds described herein can be employed together with at least one other active agent, which can be an antiviral agent. In one aspect of this embodiment, the at least one other active agent is selected from the group consisting of fusion inhibitors, entry inhibitors, protease inhibitors, polymerase inhibitors, antiviral nucleosides, such as remdesivir, GS-441524, N4-hydroxycytidine, and other compounds disclosed in U.S. Pat. No. 9,809,616, and their prodrugs, viral entry inhibitors, viral maturation inhibitors, JAK inhibitors, angiotensin-converting enzyme 2 (ACE2) inhibitors, SARS-CoV-specific human monoclonal antibodies, including CR3022, NS5A inhibitors such as daclastavir, and agents of distinct or unknown mechanism.

Umifenovir (also known as Arbidol) is a representative fusion inhibitor.

Representative entry inhibitors include Camostat, luteolin, MDL28170, SSAA09E2, SSAA09E1 (which acts as a cathepsin L inhibitor), SSAA09E3, and tetra-O-galloyl-β-D-glucose (TGG). The chemical formulae of certain of these compounds are provided below:

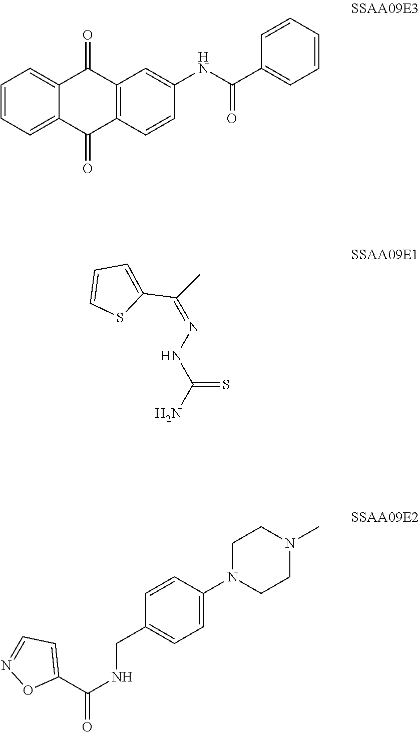 [SMILES: O=C(Nc1ccc2c(c1)C(=O)c1ccccc1C2=O)c1ccccc1 ; C/C(=N/NC(N)=S)c1cccs1 ; CN1CCN(c2ccc(CNC(=O)c3ccno3)cc2)CC1]

Other entry inhibitors include the following:

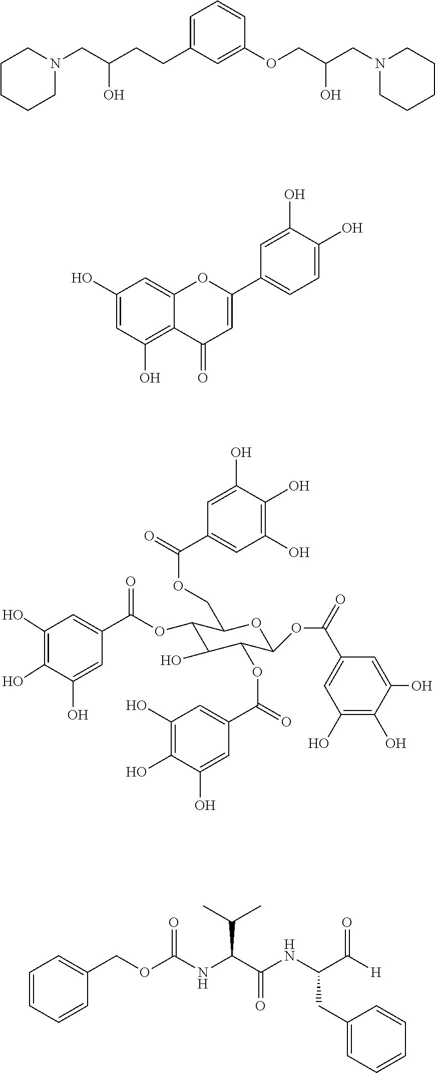 [SMILES: OC(CCc1cccc(OCC(O)CN2CCCCC2)c1)CN1CCCCC1 ; O=c1cc(-c2ccc(O)c(O)c2)oc2cc(O)cc(O)c12 ; O=C(OCC1OC(OC(=O)c2cc(O)c(O)c(O)c2)C(OC(=O)c2cc(O)c(O)c(O)c2)C(O)C1OC(=O)c1cc(O)c(O)c(O)c1)c1cc(O)c(O)c(O)c1 ; CC(C)[C@H](NC(=O)OCc1ccccc1)C(=O)N[C@H](C=O)Cc1ccccc1]

Remdesivir, Sofosbuvir, ribavirin, IDX-184 and GS-441524 have the following formulas:

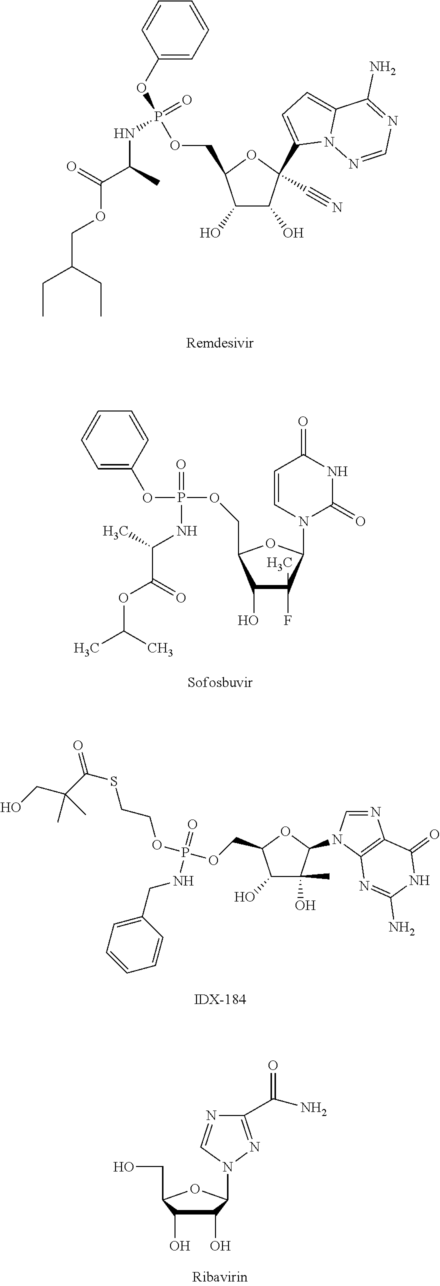

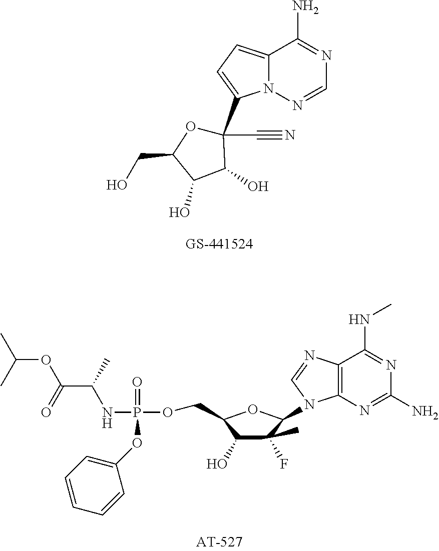 [SMILES: N#C[C@@]1(c2ccc3c(N)ncnn23)O[C@H](CO)[C@@H](O)[C@H]1O ; CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F]

Additionally, one can administer compounds which inhibit the cytokine storm, anti-coagulants and/or platelet aggregation inhibitors that address blood clots, compounds which chelate iron ions released from hemoglobin by viruses such as COVID-19, cytochrome P-450 (CYP450) inhibitors and/or NOX inhibitors.

Representative NOX inhibitors are disclosed in PCT/US2018/067674, and include AEBSF, Apocyanin, DPI, GK-136901, ML171, Plumbagin, S17834, VAS2870, VAS3947, GKT-831, GKT771, GTL003 or amido thiadiazole derivatives thereof, as described in AU2015365465, EP20140198597; and WO2015/59659, Schisandrin B, as described in CN104147001 and CN20131179455), bi-aromatic and tri-aromatic compounds described in U.S. Publication No. 2015045387, GB 20110016017, and WO201200725, methoxyflavone derivatives described in JP 2015227329, JP 20140097875, and JP 20150093939, peptides, such as NOX2ds-tat and PR-39, as described in U.S. Publication No. 2015368301, TN 2015000295, U.S. Publication No. 201514689803, U.S. Publication No. 201462013916, PCT WO 201450063, and EP 20130150187, piperazine derivatives described in U.S. Publication No. 2014194422, U.S. Pat. No. 9,428,478, U.S. Publication No. 201214123877, U.S. Publication No. 201161496161, and PCT WO 2012US41988, pyrazole derivatives disclosed in KR101280198, KR20110025151, and KR20090082518, pyrazoline dione derivatives disclosed in HK1171748, PCT WO201054329, and EP 20090171466, pyrazolo piperidine derivatives disclosed in KR20130010109, KR20130002317, EP20100153927, PCT WO201150667, EP20100153929, and PCT WO2011IB50668, pyrazolo pyridine derivatives described in KR20170026643, HK1158948, HK1141734, HK1159096, HK1159092, EP20080164857, PCT WO200954156, PCT WO200954150, EP20080164853, PCT WO200853390, U.S. Publication No. 20070896284, EP20070109555, PCT WO 200954148, EP20080164847, PCT WO200954155, and EP20080164849, quinazoline and quinoline derivatives disclosed in EP2886120, U.S. Publication No. 2014018384, U.S. Publication No. 20100407925, EP20110836947, GB20110004600, and PCT WO 201250586, tetrahydroindole derivatives disclosed in U.S. Publication No. 2010120749, U.S. Pat. No. 8,288,432, U.S. Publication No. 20080532567, EP20070109561, U.S. Publication No. 20070908414, and PCT WO 200853704, tetrahydroisoquinoline derivatives disclosed in U.S. Publication No. 2016083351, U.S. Publication No. 201414888390, U.S. Publication No. 201361818726, and PCT WO 201436402, Scopoletin, described in TW201325588 and TW20110147671, and 2,5-disubstituted benzoxazole and benzothiazole derivatives disclosed in TW201713650 and PCT WO 201554662. Representative NOX inhibitors also include those disclosed in PCT WO2011062864.

Exemplary Nox inhibitors also include 2-phenylbenzo[d]isothiazol-3(2H)-one, 2-(4-methoxyphenyl)benzo[d]isothiazol-3(2H)-one, 2-(benzo[d][1,3]dioxol-5-yl)benzo[d]isothiazol-3(2H)-one, 2-(2,4-dimethylphenyl)benzo[d]isothiazol-3(2H)-one, 2-(4-fluorophenyl)benzo[d]isothiazol-3(2H)-one, 2-(2,4-dimethylphenyl)-5-fluorobenzo[d]isothiazol-3(2H)-one, 5-fluoro-2-(4-fluorophenyl)benzo[d]isothiazol-3(2H)-one, 2-(2-chloro-6-methylphenyl)-5-fluorobenzo[d]isothiazol-3(2H)-one, 5-fluoro-2-phenylbenzo[d]isothiazol-3(2H)-one, 2-(benzo[d][1,3]dioxol-5-yl)-5-fluorobenzo[d]isothiazol-3(2H)-one, methyl 4-(3-oxobenzo[d]isothiazol-2(3H)-yl)benzoate, methyl 4-(5-fluoro-3-oxobenzo[d]isothiazol-2(3H)-yl)benzoate, ethyl 4-(3-oxobenzo[d]isothiazol-2(3H)-yl)benzoate, tert-butyl 4-(3-oxobenzo[d]isothiazol-2(3H)-yl)benzoate, methyl 2-methoxy-4-(3-oxobenzo[d]isothiazol-2(3H)-yl)benzoate, methyl 3-chloro-4-(3-oxobenzo[d]isothiazol-2(3H)-yl)benzoate, 4-(3-oxobenzo[d]isothiazol-2(3H)-yl)benzonitrile, methyl 2-(3-oxobenzo[d]isothiazol-2(3H)-yl)benzoate, 2-(4-acetylphenyl)benzo[d]isothiazol-3(2H)-one, 2-(4-nitrophenyl)benzo[d]isothiazol-3(2H)-one, 2-(4-hydroxyphenyl)benzo[d]isothiazol-3(2H)-one, methyl 6-(3-oxobenzo[d]isothiazol-2(3H)-yl)nicotinate, 6-(3-oxobenzo[d]isothiazol-2(3H)-yl)nicotinonitrile, 2-(4-(hydroxymethyl)phenyl)benzo[d]isothiazol-3(2H)-one, 2-benzylbenzo[d]isothiazol-3(2H)-one, N-methyl-4-(3-oxobenzo[d]isothiazol-2(3H)-yl)benzamide, 2-(4-hydroxyphenyl)benzo[d]isothiazol-3(2H)-one, 2-(2,4-dimethylphenyl)-1-methyl-1H-indazol-3(2H)-one, 2-(4-fluorophenyl)-1-methyl-1H-indazol-3 (2H)-one, 2-(2,4-dimethylphenyl)-1H-indazol-3(2H)-one, 1-methyl-2-phenyl-1H-indazol-3 (2H)-one, 2-(1,3,4-thiadiazol-2-yl)benzo[d]isothiazol-3(2H)-one, 2-(5-phenyl-1,3,4-thiadiazol-2-yl)benzo[d]isothiazol-3(2H)-one, 2-(5-(ethylthio)-1,3,4-thiadiazol-2-yl)benzo[d]isothiazol-3(2H)-one, 2-(5-(methylthio)-1,3,4-thiadiazol-2-yl)benzo[d]isothiazol-3(2H)-one, 5-fluoro-2-(1,3,4-thiadiazol-2-yl)benzo[d]isothiazol-3(2H)-one, 2-(5-(tert-butyl)-1,3,4-thiadiazol-2-yl)benzo[d]isothiazol-3(2H)-one, 2-(5-(4-bromophenyl)-1,3,4-thiadiazol-2-yl)benzo[d]isothiazol-3(2H)-one 2-(4-methylthiazol-2-yl)benzo[d]isothiazol-3(2H)-one, 2-(4,5-dimethylthiazol-2-yl)benzo[d]isothiazol-3(2H)-one, 2-(benzo[d][1,3]dioxol-5-yl)-4,5-difluorobenzo[d][1,2]selenazol-3(2H)-one, 2-(benzo[d][1,3]dioxol-5-yl)-5-fluorobenzo[d][1,2]selenazol-3(2H)-one, 2-(2,3-dihydrobenzo[b][1,4]dioxin-6-yl)-5-fluorobenzo[d][1,2]selenazol-3(2H)-2-(4-(1,3-dioxolan-2-yl)phenyl)benzo[d][1,2]selenazol-3(2H)-one, 2-(benzo[d][1,3]dioxol-5-yl)-6, 7-dimethoxybenzo[d][1,2]selenazol-3(2H)-one, methyl 4-(3-oxobenzo[d][1,2]selenazol-2(3H)-yl)benzoate, methyl 4-(3-oxoisothiazolo[5,4-b]pyridin-2(3H)-yl)benzoate, and ethyl 4-(3-oxoisothiazol-2(3H)-yl)benzoate, and pharmaceutically acceptable salts and prodrugs thereof.

Additional representative NOX inhibitors include:

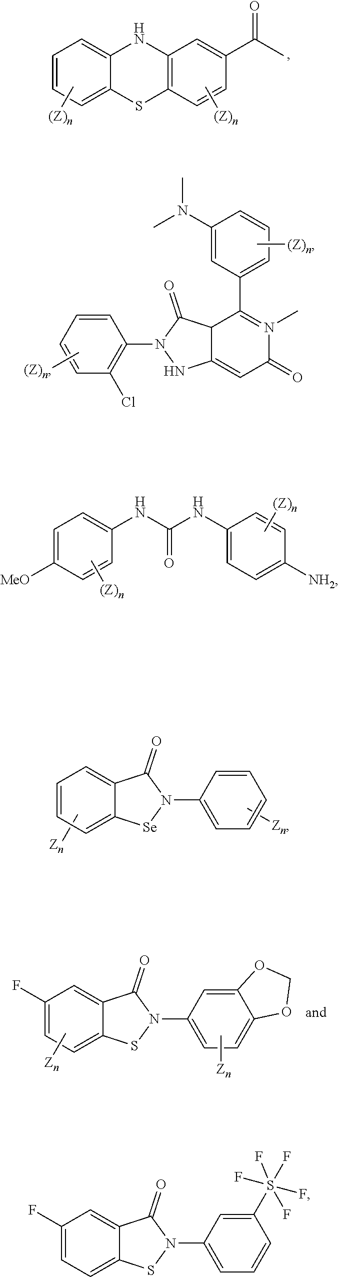

wherein
Z is selected from the group consisting of C₁₋₆ alkyl, C₁₋₆ haloalkyl, C₁₋₆ alkoxy, C₂₋₆ alkenyl, C₂₋₆ alkynyl, C₃₋₆ cycloalkyl, aryl, heteroaryl, heterocyclic, alkylaryl, arylalkyl, hydroxyl, nitro, cyano, cyanoalkyl, azido, azidoalkyl, formyl, hydrazino, halo (F, Cl, Br, or I), OR′, NHR′, SR′, S(O)R′, S(O)₂R′, S(O)₂NHR′, S(O)₂N(R′)R′, SF₅, COOR′, COR′, OCOR′, NHCOR′, N(COR′)COR′, SCOR′, OCOOR′, and NHCOOR′, wherein each R′ is independently H, a C₁₋₆ alkyl, C₁₋₆ haloalkyl, C₁₋₆ alkoxy, C₂₋₆ alkenyl, C₂₋₆ alkynyl, C₃₋₆ cycloalkyl, aryl, heteroaryl, alkylaryl, or arylalkyl, wherein the groups can be substituted with one or more substituents as defined above,
and n is an integer from 0-4,
or a pharmaceutically acceptable salt or prodrug thereof.

Specific examples of these compounds include

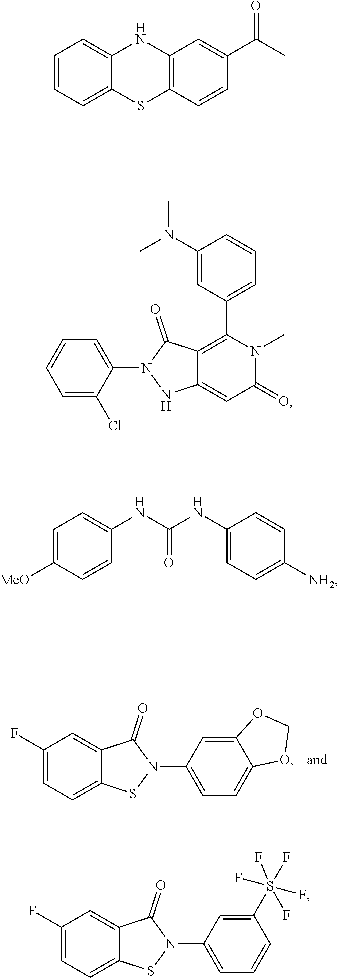 [SMILES: CC(=O)c1ccc2c(c1)Nc1ccccc1S2 ; CN(C)c1cccc(-c2c3c(=O)n(-c4ccccc4Cl)[nH]c3cc(=O)n2C)c1 ; COc1ccc(NC(=O)Nc2ccc(N)cc2)cc1 ; O=c1c2cc(F)ccc2sn1-c1ccc2c(c1)OCO2 ; "and" ; O=c1c2cc(F)ccc2sn1-c1cccc(S(F)(F)(F)(F)F)c1]

deuterated analogs thereof, or a pharmaceutically acceptable salt or prodrug thereof.

In one embodiment, the NOX inhibitor is Ebselen, Neopterin, APBA, Diapocynin, or a deuterated analog thereof, or a pharmaceutically-acceptable salt or prodrug thereof.

In another embodiment, the NOX compounds are those disclosed in PCT WO 2010/035221.

In still another embodiment, the compounds are NOX inhibitors disclosed in PCT WO 2013/068972, which are selected from the group consisting of:
4-(2-fluoro-4-methoxyphenyl)-2-(2-methoxyphenyl)-5-(pyridin-3-ylmethyl)-1H-pyrazolo[4,3-c]pyridine-3,6(2H,5H)-dione;
2-(2-chlorophenyl)-4-(4-methoxyphenyl)-5-(pyrazin-2-ylmethyl)-1H-pyrazolo[4,3-c]pyridine-3,6(2H,5H)-dione;
4-(4-chlorophenyl)-2-(2-methoxyphenyl)-5-(pyrazin-2-ylmethyl)-1H-pyrazolo[4,3-c]pyridine-3,6(2H,5H)-dione;
2-(2-chlorophenyl)-4-(2-fluoro-4-methoxyphenyl)-5-[(1-methyl-1H-pyrazol-3-yl) methyl]-1H-pyrazolo[4,3-c]pyridine-3,6(2H,5H)-dione;
4-(2-fluoro-5-methoxyphenyl)-2-(2-methoxyphenyl)-5-(pyridin-3-ylmethyl)-1H-pyrazolo[4,3-c]pyridine-3,6(2H,5H)-dione;
2-(2-chlorophenyl)-5-[(2-methoxypyridin-4-yl)methyl]-4-methyl-1H-pyrazolo[4,3-c]pyridine-3,6(2H,5H)-dione;
2-(2-methoxyphenyl)-4-methyl-5-(pyridin-3-ylmethyl)-1H-pyrazolo[4,3-c]pyridine-3,6(2H,5H)-dione;
4-(4-chloro-2-fluorophenyl)-2-(2-methoxyphenyl)-5-(pyridin-3-ylmethyl)-1H-pyrazolo [4,3-c] pyridine-3,6(2H,5H)-dione;
4-(5-chloro-2-fluorophenyl)-2-(2-chlorophenyl)-5-(pyridin-3-ylmethyl)-1H-pyrazolo [4,3-c]pyridine-3,6(2H,5H)-dione;
2-(2-chlorophenyl)-5-[(6-methoxypyridin-3-yl)methyl]-4-methyl-1H-pyrazolo[4,3-c]pyridine-3,6 (2H,5H)-dione;
4-(4-chloro-2-fluorophenyl)-2-(2-chlorophenyl)-5-(pyridin-3-ylmethyl)-1H-pyrazolo [4,3-c]pyridine-3,6(2H,5H)-dione;
4-(5-chloro-2-fluorophenyl)-2-(2-chlorophenyl)-5-(pyridin-4-ylmethyl)-1H-pyrazolo [4,3-c]pyridine-3,6(2H,5H)-dione;
4-(2-fluoro-5-methoxyphenyl)-2-(2-methoxyphenyl)-5-[(1-methyl-1H-pyrazo-1-3-yl) methyl]-1H-pyrazolo[4,3-c]pyridine-3,6(2H,5H)-dione;
4-(5-chloro-2-fluorophenyl)-2-(2-methoxyphenyl)-5-(pyridin-3-ylmethyl)-1H-pyrazolo [4,3-c] pyridine-3,6(2H,5H)-dione;
2-(2-chlorophenyl)-4-methyl-5-(pyridin-3-ylmethyl)-1H-pyrazolo[4,3-c]pyridine-3,6 (2H,5H)-dione;
2-(2-chlorophenyl)-4-(4-chlorophenyl)-5-(pyrazin-2-ylmethyl)-1H-pyrazolo[4,3-c]pyridine-3,6 (2H,5H)-dione;
2-(2-chlorophenyl)-4-(2-fluorophenyl)-5-(pyridin-3-ylmethyl)-1H-pyrazolo[4,3-c]pyridine-3,6(2H,5H)-dione;
2-(2-chlorophenyl)-4-(4-chlorophenyl)-5-(pyridin-4-ylmethyl)-1H-pyrazolo[4,3-c]pyridine-3,6(2H,5H)-dione;
4-(4-chloro-2-fluorophenyl)-2-(2-chlorophenyl)-5-(pyridin-4-ylmethyl)-1H-pyrazolo [4,3-c]pyridine-3,6(2H,5H)-dione;
2-(2-methoxyphenyl)-4-(3-methoxyphenyl)-5-[(1-methyl-1H-pyrazo-1-3-yl)methyl]-1H-pyrazolo[4,3-c]pyridine-3,6(2H,5H)-dione;
2-(2-chlorophenyl)-4-(2-fluoro-4-methoxyphenyl)-5-(pyridin-3-ylmethyl)-1H-pyrazolo [4,3-c]pyridine-3,6(2H,5H)-dione;
4-(2-fluoro-4-methoxyphenyl)-2-(2-methoxyphenyl)-5-[(1-methyl-1H-pyrazo-1-3-yl) methyl]-1H-pyrazolo[4,3-c]pyridine-3,6(2H,5H)-dione;
2-(2-methoxyphenyl)-4-(4-methoxyphenyl)-5-[(1-methyl-1H-pyrazo-1-3-yl)methyl]-1H-pyrazolo[4,3-c]pyridine-3,6(2H,5H)-dione;
2-(2-methoxyphenyl)-4-(3-methoxyphenyl)-5-(pyridin-3-ylmethyl)-1H-pyrazolo[4,3-c]pyridine-3,6(2H,5H)-dione;
2-(2-chlorophenyl)-4-(4-chlorophenyl)-5-(pyridin-3-ylmethyl)-1H-pyrazolo[4,3-c]pyridine-3,6(2H,5H)-dione;
4-(4-chloro-2-fluorophenyl)-2-(2-chlorophenyl)-5-[(2-methoxypyridin-4-yl)methyl]-1H-pyrazolo[4,3-c]pyridine-3,6(2H,5H)-dione;
2-(2-chlorophenyl)-4-(2-fluoro-4-methoxyphenyl)-5-(pyridin-4-ylmethyl)-1H-pyrazolo [4,3-c]pyridine-3,6(2H,5H)-dione;
2-(2-chlorophenyl)-4-(2,6-difluorophenyl)-5-(pyridin-4-ylmethyl)-1H-pyrazolo[4,3-c]pyridine-3,6(2H,5H)-dione;
2-(2-chlorophenyl)-4-(2-fluorophenyl)-5-(pyridin-4-ylmethyl)-1H-pyrazolo[4,3-c]pyridine-3,6(2H,5H)-dione;
2-(2-chlorophenyl)-4-methyl-5-[(1-methyl-1H-pyrazol-3-yl)methyl]-1H-pyrazolo[4,3-c]pyridine-3,6(2H,5H)-dione;
4-(3-chloro-2-fluorophenyl)-2-(2-chlorophenyl)-5-(pyridin-4-ylmethyl)-1H-pyrazolo [4,3-c]pyridine-3,6(2H,5H)-dione;
2-(2-chlorophenyl)-5-methyl-4-[3-(methylamino)phenyl]-1H-pyrazolo[4,3-c]pyridine-3,6(2H,5H)-dione;
2-(2-methoxyphenyl)-4-(4-methoxyphenyl)-5-(pyridin-3-ylmethyl)-1H-pyrazolo[4,3-c]pyridine-3,6(2H,5H)-dione;
2-(2-chlorophenyl)-4-(2-fluorophenyl)-5-(pyridin-2-ylmethyl)-1H-pyrazolo[4,3-c]pyridine-3,6(2H,5H)-dione;
2-(2-chlorophenyl)-4-(2,5-difluorophenyl)-5-(pyridin-4-ylmethyl)-1H-pyrazolo[4,3-c]pyridine-3,6(2H,5H)-dione;
2-(2-chlorophenyl)-4-(4-chlorophenyl)-5-(1,3-thiazol-2-ylmethyl)-1H-pyrazolo[4,3-c]pyridine-3,6(2H,5H)-dione;
2-(2-chlorophenyl)-4-[3-(dimethylamino)phenyl]-5-[(1-methyl-1H-pyrazol-3-yl) methyl]-1H-pyrazolo[4,3-c]pyridine-3,6(2H,5H)-dione;
2-(2-chlorophenyl)-4-(3,5-dichlorophenyl)-5-(pyridin-4-ylmethyl)-1H-pyrazolo[4,3-c]pyridine-3,6(2H,5H)-dione;
4-(3-chloro-2-fluorophenyl)-2-(2-chlorophenyl)-5-(pyridin-3-ylmethyl)-1H-pyrazolo [4,3-c]pyridine-3,6(2H,5H)-dione;
2-(2-chlorophenyl)-4-[3-(dimethylamino)phenyl]-5-(pyridin-3-ylmethyl)-1H-pyrazolo [4,3-c]pyridine-3,6(2H,5H)-dione;
2-(2-chlorophenyl)-4-(2,6-difluorophenyl)-5-(pyridin-3-ylmethyl)-1H-pyrazolo[4,3-c]pyridine-3,6(2H,5H)-dione;
4-(2-fluoro-5-methoxyphenyl)-2-(2-methoxyphenyl)-5-(pyrazin-2-ylmethyl)-1H-pyrazolo[4,3-c]pyridine-3,6(2H,5H)-dione;
2-(2-chlorophenyl)-4-(2,5-difluorophenyl)-5-(pyridin-3-ylmethyl)-1H-pyrazolo[4,3-c]pyridine-3,6(2H,5H)-dione; and
2-(2-chlorophenyl)-4-[3-(dimethylamino)phenyl]-5-[(1-methyl-1H-pyrazol-3-yl) methyl]-1H-pyrazolo[4,3-c]pyridine-3,6(2H,5H)-dione.

Representative CYP450 inhibitors include, but are not limited to, amiodarone, amlodipine, apigenin, aprepitant, bergamottin (grapefruit), buprenorphine, bupropion, caffeine, cafestol, cannabidiol, celecoxib, chloramphenicol, chlorphenamine, chlorpromazine, cimetidine, cinacalcet, ciprofloxacin, citalopram, clarithromycin, clemastine, clofibrate, clomipramine, clotrimazole, cobicistat, cocaine, curcumin (turmeric), cyclizine, delavirdine, desipramine, disulfiram, diltiazem, diphenhydramine, dithiocarbamate, domperidone, doxepin, doxorubicin, duloxetine, echinacea, entacapone, erythromycin, escitalopram, felbamate, fenofibrate, flavonoids (grapefruit), fluoroquinolones (e.g., ciprofloxacin), fluoxetine, fluvoxamine, fluconazole, fluvastatin, gabapentin, gemfibrozil, gestodene, halofantrine, haloperidol, hydroxyzine, imatinib, indomethacin, indinavir, interferon, isoniazid, itraconazole, JWH-018, ketoconazole, letrozole, lovastatin, levomepromazine, memantine, methylphenidate, metoclopramide, methadone, methimazole, methoxsalen, metyrapone, mibefradil, miconazole, midodrine, mifepristone, milk thistle, moclobemide, modafinil, montelukast, moclobemide, naringenin (grapefruit), nefazodone, nelfinavir, niacin, niacinamide, nicotine, nicotinamide, nilutamide, norfloxacin, orphenadrine, paroxetine, perphenazine, pilocarpine, piperine, phenylbutazone, probenecid, promethazine, proton pump inhibitors (e.g., lansoprazole, omeprazole, pantoprazole, rabeprazole), quercetin, quinidine, ranitidine, risperidone, ritonavir, saquinavir, selegiline, sertraline, star fruit, St. John's wort, sulconazole, sulfamethoxazole, sulfaphenazole, telithromycin, teniposide, terbinafine, thiazolidinediones, thioridazine, ticlopidine, tioconazole, thiotepa, trimethoprim, topiramate, tranylcypromine, tripelennamine, valerian, valproic acid, verapamil, voriconazole, zafirlukast, and zuclopenthixol.

Representative ACE-2 inhibitors include sulfhydryl-containing agents, such as alacepril, captopril (capoten), and zefnopril, dicarboxylate-containing agents, such as enalapril (vasotec), ramipril (altace), quinapril (accupril), perindopril (coversyl), lisinopril (listril), benazepril (lotensin), imidapril (tanatril), trandolapril (mavik), and cilazapril (inhibace), and phosphonate-containing agents, such as fosinopril (fositen/monopril).

For example, when used to treat or prevent infection, the active compound or its prodrug or pharmaceutically acceptable salt can be administered in combination or alternation with another antiviral agent including, but not limited to, those of the formulae above. In general, in combination therapy, effective dosages of two or more agents are administered together, whereas during alternation therapy, an effective dosage of each agent is administered serially. The dosage will depend on absorption, inactivation and excretion rates of the drug, as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

A number of agents for combination with the compounds described herein are disclosed in Ghosh et al., “Drug Development and Medicinal Chemistry Efforts Toward SARS-Coronavirus and Covid-19 Therapeutics,” ChemMedChem 10.1002/cmdc.202000223.

Nonlimiting examples of antiviral agents that can be used in combination with the compounds disclosed herein include those listed below.

### Compounds for Inhibiting the Cytokine Storm

Throughout its activation, the inflammatory response must be regulated to prevent a damaging systemic inflammation, also known as a “cytokine storm.” A number of cytokines with anti-inflammatory properties are responsible for this, such as IL-10 and transforming growth factor β (TGF-β). Each cytokine acts on a different part of the inflammatory response. For example, products of the Th2 immune response suppress the Th1 immune response and vice versa.

By resolving inflammation, one can minimize collateral damage to surrounding cells, with little or no long-term damage to the patient. Accordingly, in addition to using the compounds described herein to inhibit the viral infection, one or more compounds which inhibit the cytokine storm can be co-administered.

Compounds which inhibit the cytokine storm include compounds that target fundamental immune pathways, such as the chemokine network and the cholinergic anti-inflammatory pathway.

JAK inhibitors, such as JAK 1 and JAK 2 inhibitors, can inhibit the cytokine storm, and in some cases, are also antiviral. Representative JAK inhibitors include those disclosed in U.S. Pat. No. 10,022,378, such as Jakafi, Tofacitinib, and Baricitinib, as well as LY3009104/INCB28050, Pacritinib/SB1518, VX-509, GLPG0634, INC424, R-348, CYT387, TG 10138, AEG 3482, and pharmaceutically acceptable salts and prodrugs thereof.

Still further examples include CEP-701 (Lestaurtinib), AZD1480, INC424, R-348, CYT387, TG 10138, AEG 3482, 7-iodo-N-(4-morpholinophenyl)thieno[3,2-d]pyrimidin-2-amine, 7-(4-aminophenyl)-N-(4-morpholinophenyl)thieno[3,2-d]pyrimidin-2-amine, N-(4-(2-(4-morpholinophenylamino)thieno[3,2-d]pyrimidin-7-yl)phenyl) acrylamide, 7-(3-aminophenyl)-N-(4-morpholinophenyl)thieno[3,2-d]pyrimidin-2-amine, N-(3-(2-(4-morpholinophenylamino)thieno[3,2-d]pyrimidin-7-yl)phenyl) acrylamide, N-(4-morpholinophenyl)thieno[3,2-d]pyrimidin-2-amine, methyl 2-(4-morpholinophenylamino)thieno[3,2-d]pyrimidine-7-carboxylate, N-(4-morpholinophenyl)-5H-pyrrolo[3,2-d]pyrimidin-2-amine, 7-(4-amino-3-methoxyphenyl)-N-(4-morpholinophenyl)thieno[3,2-d]pyrimidin-2-amine, 4-(2-(4-morpholinophenylamino)thieno[3,2-d]pyrimidin-7-yl)benzene-sulfonamide, N,N-dimethyl-3-(2-(4-morpholinophenylamino)thieno[3,2-d]pyrimidin-7-yl)benzenesulfonamide, 1-ethyl-3-(2-methoxy-4-(2-(4-morpholinophenylamino)thieno[3,2-d]pyrimidin-7-yl)phenyl)urea, N-(4-(2-(4-morpholinophenylamino)thieno[3,2-d]pyrimidin-7-yl)phenyl)methanesulfonamide, 2-methoxy-4-(2-(4-morpholinophenylamino)thieno[3,2-d]pyrimidin-7-yl)phenol, 2-cyano-N-(3-(2-(4-morpholinophenylamino)thieno[3,2-d]pyrimidin-7-yl)phenyl)acetamide, N-(cyanomethyl)-2-(4-morpholinophenylamino)thieno[3,2-d]pyrimidine-7-carboxamide, N-(3-(2-(4-morpholinophenylamino)thieno[3,2-d]pyrimidin-7-yl)phenyl)methanesulfonamide, 1-ethyl-3-(4-(2-(4-morpholinophenylamino)thieno[3,2-d]pyrimidin-7-yl)-2-(trifluoromethoxy)phenyl)urea, N-(3-nitrophenyl)-7-phenylthieno[3,2-d]pyrimidin-2-amine, 7-iodo-N-(3-nitrophenyl)thieno[3,2-d]pyrimidin-2-amine, N1-(7-(2-ethylphenyl)thieno[3,2-d]pyrimidin-2-yl)benzene-1,3-diamine, N-tert-butyl-3-(2-(4-morpholinophenylamino)thieno[3,2-d]pyrimidin-7-yl)benzenesulfonamide, N1-(7-iodothieno[3,2-d]pyrimidin-2-yl)benzene-1,3-diamine, 7-(4-amino-3-(trifluoromethoxy)phenyl)-N-(4-morpholinophenyl)thieno[3,2-d]pyrimidin-2-amine, 7-(2-ethylphenyl)-N-(4-morpholinophenyl)thieno[3,2-d]pyrimidin-2-amine, N-(3-(2-(4-morpholinophenylamino)thieno[3,2-d]pyrimidin-7-yl)phenyl)acetamide, N-(cyanomethyl)-N-(3-(2-(4-morpholinophenylamino)thieno[3,2-d]pyrimidin-7-yl)phenyl)methanesulfonamide, N-(cyanomethyl)-N-(4-(2-(4-morpholinophenylamino)thieno[3,2-d]pyrimidin-7-yl)phenyl)methanesulfonamide, N-(3-(5-methyl-2-(4-morpholinophenylamino)-5H-pyrrolo[3,2-d]pyrimidin-7-yl)phenyl)methanesulfonamide, 4-(5-methyl-2-(4-morpholinophenylamino)-5H-pyrrolo[3,2-d]pyrimidin-7-yl)benzenesulfonamide, N-(4-(5-methyl-2-(4-morpholinophenylamino)-5H-pyrrolo[3,2-d]pyrimidin-7-yl)phenyl)methanesulfonamide, 7-iodo-N-(4-morpholinophenyl)-5H-pyrrolo[3,2-d]pyrimidin-2-amine, 7-(2-isopropylphenyl)-N-(4-morpholinophenyl)thieno[3,2-d]pyrimidin-2-amine, 7-bromo-N-(4-morpholinophenyl)thieno[3,2-d]pyrimidin-2-amine, N7-(2-isopropylphenyl)-N2-(4-morpholinophenyl)thieno[3,2-d]pyrimidine-2,7-diamine, N7-(4-isopropylphenyl)-N2-(4-morpholinophenyl)thieno[3,2-d]pyrimidine-2,7-diamine, 7-(5-amino-2-methylphenyl)-N-(4-morpholinophenyl)thieno[3,2-d]pyrimidin-2-amine, N-(cyanomethyl)-4-(2-(4-morpholinophenylamino)thieno[3,2-d]pyrimidin-7-yl)benzamide, 7-iodo-N-(3-morpholinophenyl)thieno[3,2-d]pyrimidin-2-amine, 7-(4-amino-3-nitrophenyl)-N-(4-morpholinophenyl)thieno[3,2-d]pyrimidin-2-amine, 7-(2-methoxypyridin-3-yl)-N-(4-morpholinophenyl)thieno[3,2-d]pyrimidin-2-amine, (3-(7-iodothieno[3,2-d]pyrimidin-2-ylamino)phenyl)methanol, N-tert-butyl-3-(2-(3-morpholinophenylamino)thieno[3,2-d]pyrimidin-7-yl)benzenesulfonamide, N-tert-butyl-3-(2-(3-(hydroxymethyl)phenylamino)thieno[3,2-d]pyrimidin-7-yl)benzenesulfonamide, N-(4-morpholinophenyl)-7-(4-nitrophenylthio)-5H-pyrrolo[3,2-d]pyrimidin-2-amine, N-tert-butyl-3-(2-(3,4,5-trimethoxyphenylamino)thieno[3,2-d]pyrimidin-7-yl)benzenesulfonamide, 7-(4-amino-3-nitrophenyl)-N-(3,4-dimethoxyphenyl)thieno[3,2-d]pyrimidin-2-amine, N-(3,4-dimethoxyphenyl)-7-(2-methoxypyridin-3-yl)thieno[3,2-d]pyrimidin-2-amine, N-tert-butyl-3-(2-(3,4-dimethoxyphenylamino)thieno[3,2-d]pyrimidin-7-yl)benzenesulfonamide, 7-(2-aminopyrimidin-5-yl)-N-(3,4-dimethoxyphenyl)thieno[3,2-d]pyrimidin-2-amine, N-(3,4-dimethoxyphenyl)-7-(2,6-dimethoxypyridin-3-yl)thieno[3,2-d]-pyrimidin-2-amine, N-(3,4-dimethoxyphenyl)-7-(2,4-dimethoxypyrimidin-5-yl)thieno[3,2-d]pyrimidin-2-amine, 7-iodo-N-(4-(morpholinomethyl)phenyl)thieno[3,2-d]pyrimidin-2-amine, N-tert-butyl-3-(2-(4-(morpholinomethyl)phenylamino)thieno[3,2-d]pyrimidin-7-yl)benzenesulfonamide, 2-cyano-N-(4-methyl-3-(2-(4-morpholinophenylamino)thieno[3,2-d]pyrimidin-7-yl)phenyl)acetamide, ethyl 3-(2-(4-morpholinophenylamino)thieno[3,2-d]pyrimidin-7-yl)benzoate, 7-bromo-N-(4-(2-(pyrrolidin-1-yl)ethoxy)phenyl)thieno[3,2-d]pyrimidin-2-amine, N-(3-(2-(4-(2-(pyrrolidin-1-yl)ethoxy)phenylamino)thieno[3,2-d]pyrimidin-7-yl)phenyl)acetamide, N-(cyanomethyl)-3-(2-(4-morpholinophenylamino)thieno[3,2-d]pyrimidin-7-yl)benzamide, N-tert-butyl-3-(2-(4-morpholinophenylamino)thieno[3,2-d]pyrimidin-7-yl)benzamide, N-tert-butyl-3-(2-(4-(1-ethylpiperidin-4-yloxy)phenylamino)thieno-[3,2-d]pyrimidin-7-yl)benzenesulfonamide, tert-butyl-4-(2-(4-(morpholinomethyl)phenylamino)thieno[3,2-d]pyrimidin-7-yl)-1H-pyrazole-1-carboxylate, 7-bromo-N-(4-((4-ethylpiperazin-1-yl)methyl)phenyl)thieno[3,2-d]pyrimidin-2-amine, N-tert-butyl-3-(2-(4-((4-ethylpiperazin-1-yl)methyl)phenylamino)-thieno[3,2-d]pyrimidin-7-yl)benzenesulfonamide, N-(4-((4-ethylpiperazin-1-yl)methyl)phenyl)-7-(1H-pyrazol-4-yl)thieno[3,2-d]pyrimidin-2-amine, N-(cyanomethyl)-3-(2-(4-(morpholinomethyl)phenylamino)thieno[3,2-d]pyrimidin-7-yl)benzamide, N-tert-butyl-3-(2-(4-(2-(pyrrolidin-1-yl)ethoxy)phenylamino)thieno[3,2-d]-pyrimidin-7-yl)benzenesulfonamide, tert-butyl pyrrolidin-1-yl)ethoxy)phenylamino)thieno[3,2-d]pyrimidin-7-yl)benzylcarbamate, 3-(2-(4-(2-(pyrrolidin-1-yl)ethoxy)phenylamino)thieno[3,2-d]pyrimidin-7-yl)benzenesulfonamide, 7-(3-chloro-4-fluorophenyl)-N-(4-(2-(pyrrolidin-1-yl)ethoxy)phenyl)thieno-[3,2-d]pyrimidin-2-amine, tert-butyl 4-(2-(4-(1-ethylpiperidin-4-yloxy)phenylamino)thieno[3,2-d]pyrimidin-7-yl-)-1H-pyrazole-1-carboxylate, 7(benzo[d][1,3]dioxol-5-yl)-N-(4-(morpholinomethyl)phenyl)thieno[3,2-d]pyrimidin-2-amine, tert-butyl 5-(2-(4-(morpholinomethyl)phenylamino)thieno[3,2-d]pyrimidin-7-yl)-1H-indole-1-carboxylate, 7-(2-aminopyrimidin-5-yl)-N-(4-(morpholinomethyl)phenyl)thieno[3,2-d]pyrimidin-2-amine, tert-butyl 4-(2-(-4-(morpholinomethyl)phenylamino)thieno[3,2-d]pyrimidin-7-yl)-5, 6-dihydropyridine-1(2H)-carboxylate, tert-butyl morpholinomethyl)phenylamino)thieno[3,2-d]pyrimidin-7-yl)benzylcarbamate, N-(3-(2-(4-(morpholinomethyl)phenylamino)thieno[3,2-d]pyrimidin-7-yl)phenyl)acetamide, N-(4-(2-(4-(morpholinomethyl)phenylamino)thieno[3,2-d]pyrimidin-7-yl)phenyl)acetamide, N-(3-(2-(4-(morpholinomethyl)phenylamino)thieno[3,2-d]pyrimidin-7-yl)phenyl)methanesulfonamide, 7-(4-(4-methylpiperazin-1-yl)phenyl)-N-(4-(morpholinomethyl)phenyl)thieno-[3,2-d]pyrimidin-2-amine, N-(2-methoxy-4-(2-(4-(morpholinomethyl)phenylamino)thieno[3,2-d]pyrimidin-7-yl)phenyl)acetamide, 7-bromo-N-(3,4,5-trimethoxyphenyl)thieno[3,2-d]pyrimidin-2-amine, (3-(2-(3,4,5-trimethoxyphenylamino)thieno[3,2-d]pyrimidin-7-yl)phenyl)methanol, (4-(2-(3,4,5-trimethoxyphenylamino)thieno[3,2-d]pyrimidin-7-yl)phen-yl)methanol, (3-(2-(4-morpholinophenylamino)thieno[3,2-d]pyrimidin-7-yl)phenyl)methano-1, (4-(2-(4-morpholinophenylamino)thieno[3,2-d]pyrimidin-7-yl)phenyl)methanol, N-(pyrrolidin-1-yl)ethoxy)phenylamino)thieno[3,2-d]pyrimidin-7-yl)benzyl)methanesulfonamide, tert-butyl morpholinomethyl)phenylamino)thieno[3,2-d]pyrimidin-7-yl)benzylcarbamate, N-(4-(morpholinomethyl)phenyl)-7-(3-(piperazin-1-yl)phenyl)thieno[3,2-d]pyrimidin-2-amine, 7-(6-(2-morpholinoethylamino)pyridin-3-yl)-N-(3,4,5-trimethoxyphenyl)thieno[3,2-d]pyrimidin-2-amine, 7-(2-ethylphenyl)-N-(4-(2-(pyrrolidin-1-yl)ethoxy)phenyl)thieno[3,2-d]pyrimidin-2-amine, 7-(4-(aminomethyl)phenyl)-N-(4-(morpholinomethyl)phenyl)thieno[3,2-d]pyrimidin-2-amine, N-(4-(1-ethylpiperidin-4-yloxy)phenyl)-7-(1H-pyrazol-4-yl)thieno[3,2-d]pyrimidin-2-amine, N-(2,4-dimethoxyphenyl)-7-phenylthieno[3,2-d]pyrimidin-2-amine, 7-bromo-N-(3,4-dimethoxyphenyl)thieno[3,2-d]pyrimidin-2-amine, N-(3,4-dimethoxyphenyl)-7-phenylthieno[3,2-d]pyrimidin-2-amine, and pharmaceutically acceptable salts and prodrugs thereof.

HMGB1 antibodies and COX-2 inhibitors can be used, which downregulate the cytokine storm. Examples of such compounds include Actemra (Roche). Celebrex (celecoxib), a COX-2 inhibitor, can be used. IL-8 (CXCL8) inhibitors can also be used.

Chemokine receptor CCR2 antagonists, such as PF-04178903 can reduce pulmonary immune pathology.

Selective α7Ach receptor agonists, such as GTS-21 (DMXB-A) and CNI-1495, can be used. These compounds reduce TNF-α. The late mediator of sepsis, HMGB1, downregulates IFN-γ pathways, and prevents the LPS-induced suppression of IL-10 and STAT 3 mechanisms.

### Compounds for Treating or Preventing Blood Clots

Viruses that cause respiratory infections, including Coronaviruses such as Covid-19, can be associated with pulmonary blood clots, and blood clots that can also do damage to the heart.

The compounds described herein can be co-administered with compounds that inhibit blood clot formation, such as blood thinners, or compounds that break up existing blood clots, such as tissue plasminogen activator (TPA), Integrilin (eptifibatide), abciximab (ReoPro) or tirofiban (Aggrastat).

Blood thinners prevent blood clots from forming, and keep existing blood clots from getting larger. There are two main types of blood thinners. Anticoagulants, such as heparin or warfarin (also called Coumadin), slow down biological processes for producing clots, and antiplatelet aggregation drugs, such as Plavix, aspirin, prevent blood cells called platelets from clumping together to form a clot.

By way of example, Integrilin® is typically administered at a dosage of 180 meg/kg intravenous bolus administered as soon as possible following diagnosis, with 2 meg/kg/min continuous infusion (following the initial bolus) for up to 96 hours of therapy.

Representative platelet aggregation inhibitors include glycoprotein IIB/IIIA inhibitors, phosphodiesterase inhibitors, adenosine reuptake inhibitors, and adenosine diphosphate (ADP) receptor inhibitors. These can optionally be administered in combination with an anticoagulant.

Representative anti-coagulants include coumarins (vitamin K antagonists), heparin and derivatives thereof, including unfractionated heparin (UFH), low molecular weight heparin (LMWH), and ultra-low-molecular weight heparin (ULMWH), synthetic pentasaccharide inhibitors of factor Xa, including Fondaparinux, Idraparinux, and Idrabiotaparinux, directly acting oral anticoagulants (DAOCs), such as dabigatran, rivaroxaban, apixaban, edoxaban and betrixaban, and antithrombin protein therapeutics/thrombin inhibitors, such as bivalent drugs hirudin, lepirudin, and bivalirudin and monovalent argatroban.

Representative platelet aggregation inhibitors include pravastatin, Plavix (clopidogrel bisulfate), Pletal (cilostazol), Effient (prasugrel), Aggrenox (aspirin and dipyridamole), Brilinta (ticagrelor), caplacizumab, Kengreal (cangrelor), Persantine (dipyridamole), Ticlid (ticlopidine), Yosprala (aspirin and omeprazole).

### Small Molecule Covalent CoV 3CLpro Inhibitors

Representative small molecule covalent CoV 3CLpro inhibitors include the following compounds:

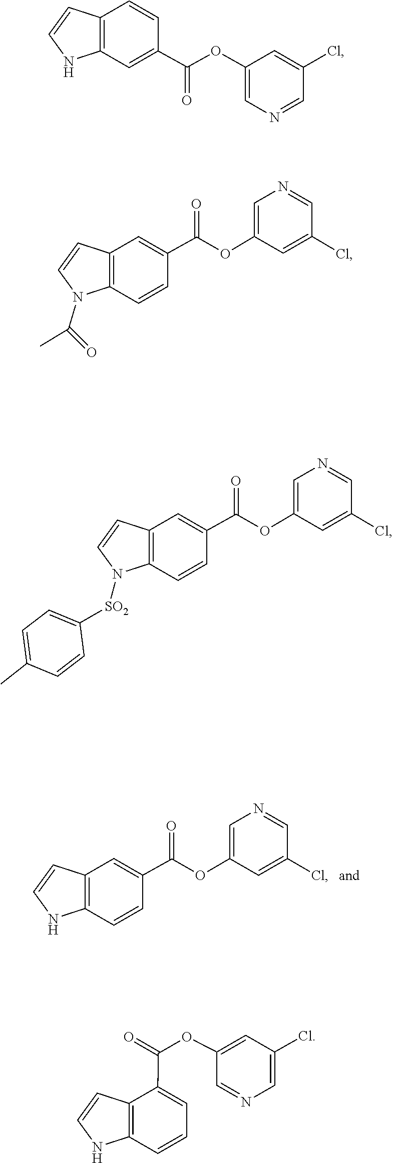 [SMILES: O=C(Oc1cncc(Cl)c1)c1ccc2cc[nH]c2c1 ; CC(=O)n1ccc2cc(C(=O)Oc3cncc(Cl)c3)ccc21 ; Cc1ccc(S(=O)(=O)n2ccc3cc(C(=O)Oc4cncc(Cl)c4)ccc32)cc1 ; O=C(Oc1cncc(Cl)c1)c1ccc2[nH]ccc2c1 ; "and" ; O=C(Oc1cncc(Cl)c1)c1cccc2[nH]ccc12]

### Non-Covalent CoV 3CLpro Inhibitors

Representative non-covalent CoV 3CLpro inhibitors include the following:

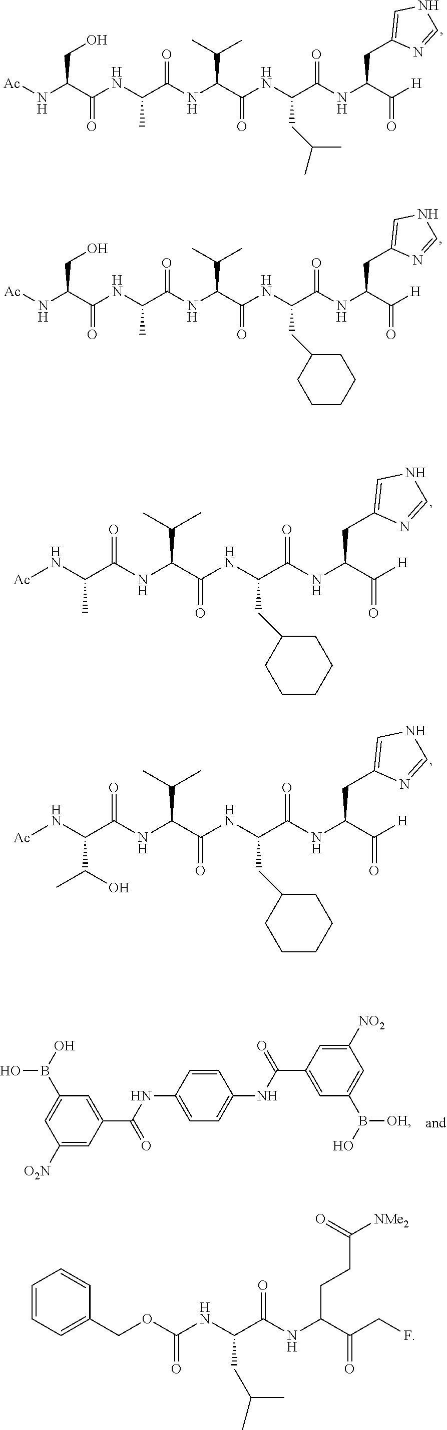

### SARS-CoV PLpro Inhibitors

Representative SARS-Cov PLpro inhibitors include the following:

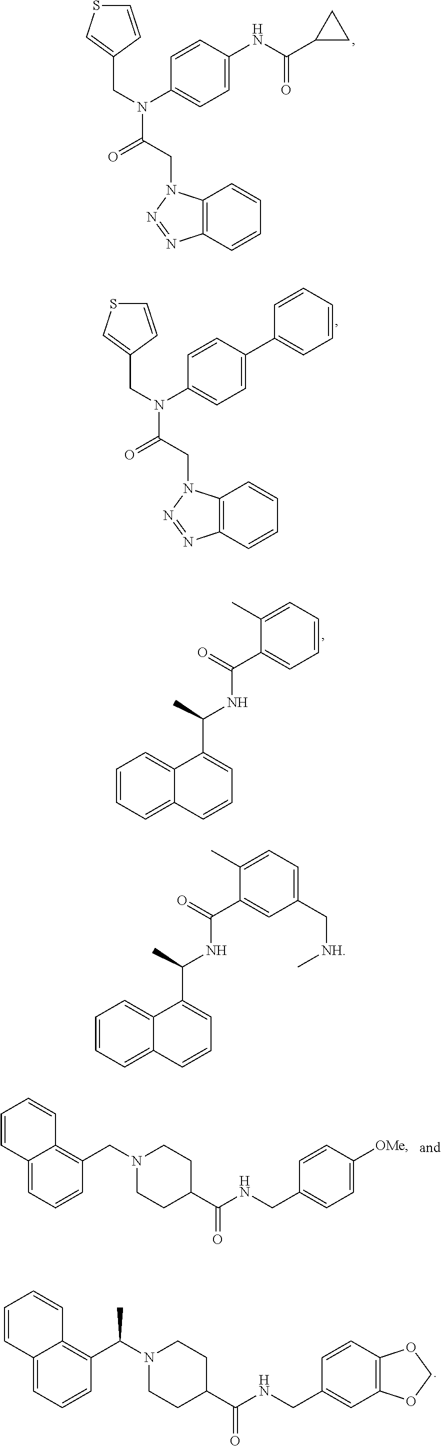

Additional compounds include the following:

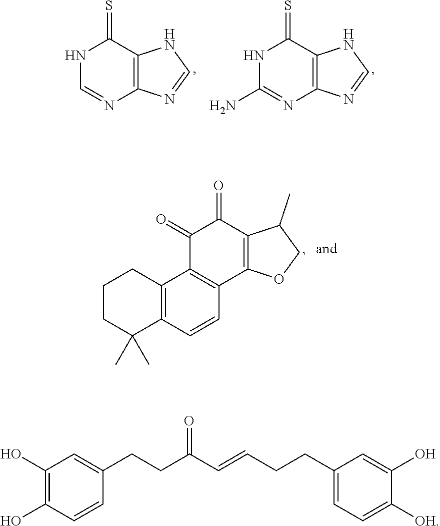 [SMILES: S=c1[nH]cnc2nc[nH]c12 ; Nc1nc2nc[nH]c2c(=S)[nH]1 ; CC1COC2=C1C(=O)C(=O)c1c2ccc2c1CCCC2(C)C ; O=C(/C=C/CCc1ccc(O)c(O)c1)CCc1ccc(O)c(O)c1]

Additional Compounds that can be Used

Additional compounds and compound classes that can be used in combination therapy include the following: Antibodies, including monoclonal antibodies (mAb), Arbidol (umifenovir), Actemra (tocilizumab), APNO1 (Aperion Biologics), ARMS-1 (which includes Cetylpyridinium chloride (CPC)), ASC09 (Ascletis Pharma), AT-001 (Applied Therapeutics Inc.) and other aldose reductase inhibitors (ARI), ATYR1923 (aTyr Pharma, Inc.), Aviptadil (Relief Therapeutics), Azvudine, Bemcentinib, BLD-2660 (Blade Therapeutics), Bevacizumab, Brensocatib, Calquence (acalabrutinib), Camostat mesylate (a TMPRSS2 inhibitor), Camrelizumab, CAP-1002 (Capricor Therapeutics), CD24Fcm, Clevudine, (OncoImmune), CM4620-IE (CalciMedica Inc., CRAC channel inhibitor), Colchicine, convalescent plasma, CYNK-001 (Sorrento Therapeutics), DAS181 (Ansun Pharma), Desferal, Dipyridamole (Persantine), Dociparstat sodium (DSTAT), Duvelisib, Eculizumab, EIDD-2801 (Ridgeback Biotherapeutics), Emapalumab, Fadraciclib (CYC065) and seliciclib (roscovitine) (Cyclin-dependent kinase (CDK) inhibitors), Farxiga (dapagliflozin), Favilavir/Favipiravir/T-705/Avigan, Galidesivir, Ganovo (danoprevir), Gilenya (fingolimod) (sphingosine 1-phosphate receptor modulator), Gimsilumab, IFX-1, Ilaris (canakinumab), intravenous immunoglobulin, Ivermectin (importin a/P inhibitor), Kaletra/Aluvia (lopinavir/ritonavir), Kevzara (sarilumab), Kineret (anakinra), LAU-7b (fenretinide), Lenzilumab, Leronlimab (PRO 140), LY3127804 (an anti-Ang2 antibody), Leukine (sargramostim, a granulocyte macrophage colony stimulating factor), Losartan, Valsartan, and Telmisartan (Angiotensin II receptor antagonists), Meplazumab, Metablok (LSALT peptide, a DPEP1 inhibitor), Methylprednisolone and other corticosteroids, MN-166 (ibudilast, Macrophage migration inhibitory factor (MIF) inhibitor), MRx-4DP0004 (a strain of Bifidobacterium breve, 4D Pharma), Nafamostat (a serine protease inhibitor), Neuraminidase inhibitors like Tamiflu (oseltamivir), Nitazoxanide (nucleocapsid (N) protein inhibitor), Nivolumab, OT-101 (Mateon), Novaferon (man-made Interferon), Opaganib (yeliva) (Sphingosine kinase-2 inhibitor), Otilimab, PD-1 blocking antibody, peginterferons, such as peginterferon lambda, Pepcid (famotidine), Piclidenoson (A3 adenosine receptor agonist), Prezcobix (darunavir), PUL-042 (Pulmotect, Inc., toll-like receptor (TLR) binder), Rebif (interferon beta-la), RHB-107 (upamostat) (serine protease inhibitor, RedHill Biopharma Ltd.), Selinexor (selective inhibitor of nuclear export (SINE)), SNG001 (Synairgen, inhaled interferon beta-la), Solnatide, stem cells, including mesenchymal stem cells, MultiStem (Athersys), and PLX (Pluristem Therapeutics), Sylvant (siltuximab), Thymosin, TJM2 (TJ003234), Tradipitant (neurokinin-1 receptor antagonist), Truvada (emtricitabine and tenofovir), Ultomiris (ravulizumab-cwvz), Vazegepant (CGRP receptor antagonist or blocker), and Xofluza (baloxavir marboxil).

### Repurposed Antiviral Agents

A number of pharmaceutical agents, including agents active against other viruses, have been evaluated against Covid-19, and found to have activity. Any of these compounds can be combined with the compounds described herein. Representative compounds include lopinavir, ritonavir, niclosamide, promazine, PNU, UC2, cinanserin (SQ 10,643), Calmidazolium (C3930), tannic acid, 3-isotheaflavin-3-gallate, theaflavin-3,3′-digallate, glycyrrhizin, S-nitroso-N-acetylpenicillamine, nelfinavir, niclosamide, chloroquine, hydroxychloroquine, 5-benzyloxygramine, ribavirin, Interferons, such as Interferon (IFN)-α, IFN-β, and pegylated versions thereof, as well as combinations of these compounds with ribavirin, chlorpromazine hydrochloride, triflupromazine hydrochloride, gemcitabine, imatinib mesylate, dasatinib, and imatinib.

### VIII. Pharmaceutical Compositions

Hosts, including but not limited to humans, infected with a Coronviridae virus, or the other viruses described, herein can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

A preferred dose of the compound for will be in the range of between about 0.01 and about 10 mg/kg, more generally, between about 0.1 and 5 mg/kg, and, preferably, between about 0.5 and about 2 mg/kg, of body weight of the recipient per day, until the patient has recovered. In some cases, a compound may be administered at a dosage of up to 10 μM, which might be considered a relatively high dose if administered for an extended period of time, but which can be acceptable when administered for the duration of an infection with one or more of the viruses described herein, which is typically on the order of several days to several weeks.

The effective dosage range of the pharmaceutically acceptable salts and prodrugs can be calculated based on the weight of the parent compound to be delivered. If the salt or prodrug exhibits activity in itself, the effective dosage can be estimated as above using the weight of the salt or prodrug, or by other means known to those skilled in the art.

The compound is conveniently administered in unit any suitable dosage form, including but not limited to but not limited to one containing 7 to 600 mg, preferably 70 to 600 mg of active ingredient per unit dosage form. An oral dosage of 5-400 mg is usually convenient.

The concentration of active compound in the drug composition will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient can be administered at once, or can be divided into a number of smaller doses to be administered at varying intervals of time.

A preferred mode of administration of the active compound is oral. Oral compositions will generally include an inert diluent or an edible carrier. They can be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, unit dosage forms can contain various other materials that modify the physical form of the dosage unit, for example, coatings of sugar, shellac, or other enteric agents.

The compound can be administered as a component of an elixir, suspension, syrup, wafer, chewing gum or the like. A syrup can contain, in addition to the active compound(s), sucrose as a sweetening agent and certain preservatives, dyes and colorings and flavors.

The compound or a pharmaceutically acceptable prodrug or salts thereof can also be mixed with other active materials that do not impair the desired action, or with materials that supplement the desired action, such as antibiotics, antifungals, anti-inflammatories or other antiviral compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents, such as ethylenediaminetetraacetic acid; buffers, such as acetates, citrates or phosphates, and agents for the adjustment of tonicity, such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

### Transdermal Formulations

In some embodiments, the compositions are present in the form of transdermal formulations, such as that used in the FDA-approved agonist rotigitine transdermal (Neupro patch). Another suitable formulation is that described in U.S. Publication No. 20080050424, entitled “Transdermal Therapeutic System for Treating Parkinsonism.” This formulation includes a silicone or acrylate-based adhesive, and can include an additive having increased solubility for the active substance, in an amount effective to increase dissolving capacity of the matrix for the active substance.

The transdermal formulations can be single-phase matrices that include a backing layer, an active substance-containing self-adhesive matrix, and a protective film to be removed prior to use. More complicated embodiments contain multiple-layer matrices that may also contain non-adhesive layers and control membranes. If a polyacrylate adhesive is used, it can be crosslinked with multivalent metal ions such as zinc, calcium, aluminum, or titanium ions, such as aluminum acetylacetonate and titanium acetylacetonate.

When silicone adhesives are used, they are typically polydimethylsiloxanes. However, other organic residues such as, for example, ethyl groups or phenyl groups may in principle be present instead of the methyl groups. Because the active compounds are amines, it may be advantageous to use amine-resistant adhesives. Representative amine-resistant adhesives are described, for example, in EP 0 180 377.

Representative acrylate-based polymer adhesives include acrylic acid, acrylamide, hexylacrylate, 2-ethylhexylacrylate, hydroxyethylacrylate, octylacrylate, butylacrylate, methylacrylate, glycidylacrylate, methacrylic acid, methacrylamide, hexylmethacrylate, 2-ethylhexylmethacrylate, octylmethacrylate, methylmethacrylate, glycidylmethacrylate, vinylacetate, vinylpyrrolidone, and combinations thereof.

The adhesive must have a suitable dissolving capacity for the active substance, and the active substance most be able to move within the matrix, and be able to cross through the contact surface to the skin. Those of skill in the art can readily formulate a transdermal formulation with appropriate transdermal transport of the active substance.

Certain pharmaceutically acceptable salts tend to be more preferred for use in transdermal formulations, because they can help the active substance pass the barrier of the stratum corneum. Examples include fatty acid salts, such as stearic acid and oleic acid salts. Oleate and stearate salts are relatively lipophilic, and can even act as a permeation enhancer in the skin.

Permeation enhancers can also be used. Representative permeation enhancers include fatty alcohols, fatty acids, fatty acid esters, fatty acid amides, glycerol or its fatty acid esters, N-methylpyrrolidone, terpenes such as limonene, alpha-pinene, alpha-terpineol, carvone, carveol, limonene oxide, pinene oxide, and 1,8-eucalyptol.

The patches can generally be prepared by dissolving or suspending the active agent in ethanol or in another suitable organic solvent, then adding the adhesive solution with stirring. Additional auxiliary substances can be added either to the adhesive solution, the active substance solution or to the active substance-containing adhesive solution. The solution can then be coated onto a suitable sheet, the solvents removed, a backing layer laminated onto the matrix layer, and patches punched out of the total laminate.

### Nanoparticulate Compositions

The compounds described herein can also be administered in the form of nanoparticulate compositions. In one embodiment, controlled release nanoparticulate formulations comprise a nanoparticulate active agent to be administered and a rate-controlling polymer which prolongs the release of the agent following administration. In this embodiment, the compositions can release the active agent, following administration, for a time period ranging from about 2 to about 24 hours or up to 30 days or longer. Representative controlled release formulations including a nanoparticulate form of the active agent are described, for example, in U.S. Pat. No. 8,293,277.

Nanoparticulate compositions can comprise particles of the active agents described herein, having a non-crosslinked surface stabilizer adsorbed onto, or associated with, their surface.

The average particle size of the nanoparticulates is typically less than about 800 nm, more typically less than about 600 nm, still more typically less than about 400 nm, less than about 300 nm, less than about 250 nm, less than about 100 nm, or less than about 50 nm. In one aspect of this embodiment, at least 50% of the particles of active agent have an average particle size of less than about 800, 600, 400, 300, 250, 100, or 50 nm, respectively, when measured by light scattering techniques.

A variety of surface stabilizers are typically used with nanoparticulate compositions to prevent the particles from clumping or aggregating. Representative surface stabilizers are selected from the group consisting of gelatin, lecithin, dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol, polyvinylpyrrolidone, tyloxapol, poloxamers, poloxamines, poloxamine 908, dialkylesters of sodium sulfosuccinic acid, sodium lauryl sulfate, an alkyl aryl polyether sulfonate, a mixture of sucrose stearate and sucrose distearate, p-isononylphenoxypoly-(glycidol), SA90HCO, decanoyl-N-methylglucamide, n-decyl-D-glucopyranoside, n-decyl-D-maltopyranoside, n-dodecyl-D-glucopyranoside, n-dodecyl-D-maltoside, heptanoyl-N-methylglucamide, n-heptyl-D-glucopyranoside, n-heptyl-D-thioglucoside, n-hexyl-D-glucopyranoside, nonanoyl-N-methylglucamide, n-nonyl-D-glucopyranoside, octanoyl-N-methylglucamide, n-octyl-D-glucopyranoside, and octyl-D-thioglucopyranoside. Lysozymes can also be used as surface stabilizers for nanoparticulate compositions. Certain nanoparticles such as poly(lactic-co-glycolic acid) (PLGA)-nanoparticles are known to target the liver when given by intravenous (IV) or subcutaneously (SQ).

Representative rate controlling polymers into which the nanoparticles can be formulated include chitosan, polyethylene oxide (PEO), polyvinyl acetate phthalate, gum arabic, agar, guar gum, cereal gums, dextran, casein, gelatin, pectin, carrageenan, waxes, shellac, hydrogenated vegetable oils, polyvinylpyrrolidone, hydroxypropyl cellulose (HPC), hydroxyethyl cellulose (HEC), hydroxypropyl methylcelluose (HPMC), sodium carboxymethylcellulose (CMC), poly(ethylene) oxide, alkyl cellulose, ethyl cellulose, methyl cellulose, carboxymethyl cellulose, hydrophilic cellulose derivatives, polyethylene glycol, polyvinylpyrrolidone, cellulose acetate, cellulose acetate butyrate, cellulose acetate phthalate, cellulose acetate trimellitate, polyvinyl acetate phthalate, hydroxypropylmethyl cellulose phthalate, hydroxypropylmethyl cellulose acetate succinate, polyvinyl acetaldiethylamino acetate, poly(alkylmethacrylate), poly(vinyl acetate), polymers derived from acrylic or methacrylic acid and their respective esters, and copolymers derived from acrylic or methacrylic acid and their respective esters.

Methods of making nanoparticulate compositions are described, for example, in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive nanoparticulate compositions having cationic surface stabilizers;” U.S. Pat. No. 6,431,478 for “Small Scale Mill;” and U.S. Pat. No. 6,432,381 for “Methods for targeting drug delivery to the upper and/or lower gastrointestinal tract,” all of which are specifically incorporated by reference. In addition, U.S. Patent Application No. 20020012675 A1, published on Jan. 31, 2002, for “Controlled Release Nanoparticulate Compositions,” describes nanoparticulate compositions, and is specifically incorporated by reference.

Amorphous small particle compositions are described, for example, in U.S. Pat. No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.”

Certain nanoformulations can enhance the absorption of drugs by releasing drug into the lumen in a controlled manner, thus reducing solubility issues. The intestinal wall is designed to absorb nutrients and to act as a barrier to pathogens and macromolecules. Small amphipathic and lipophilic molecules can be absorbed by partitioning into the lipid bilayers and crossing the intestinal epithelial cells by passive diffusion, while nanoformulation absorption may be more complicated because of the intrinsic nature of the intestinal wall. The first physical obstacle to nanoparticle oral absorption is the mucus barrier which covers the luminal surface of the intestine and colon. The mucus barrier contains distinct layers and is composed mainly of heavily glycosylated proteins called mucins, which have the potential to block the absorption of certain nanoformulations. Modifications can be made to produce nanoformulations with increased mucus-penetrating properties (Ensign et al., “Mucus penetrating nanoparticles: biophysical tool and method of drug and gene delivery,” Adv Mater 24: 3887-3894 (2012)).

Once the mucus coating has been traversed, the transport of nanoformulations across intestinal epithelial cells can be regulated by several steps, including cell surface binding, endocytosis, intracellular trafficking and exocytosis, resulting in transcytosis (transport across the interior of a cell) with the potential involvement of multiple subcellular structures. Moreover, nanoformulations can also travel between cells through opened tight junctions, defined as paracytosis. Non-phagocytic pathways, which involve clathrin-mediated and caveolae-mediated endocytosis and macropinocytosis, are the most common mechanisms of nanoformulation absorption by the oral route.

Non-oral administration can provide various benefits, such as direct targeting to the desired site of action and an extended period of drug action. Transdermal administration has been optimized for nanoformulations, such as solid lipid nanoparticles (SLNs) and NEs, which are characterized by good biocompatibility, lower cytotoxicity and desirable drug release modulation (Cappel and Kreuter, “Effect of nanoparticles on transdermal drug delivery. J Microencapsul 8: 369-374 (1991)). Nasal administration of nanoformulations allows them to penetrate the nasal mucosal membrane, via a transmucosal route by endocytosis or via a carrier- or receptor-mediated transport process (Illum, “Nanoparticulate systems for nasal delivery of drugs: a real improvement over simple systems?” J. Pharm. Sci 96: 473-483 (2007)), an example of which is the nasal administration of chitosan nanoparticles of tizanidine to increase brain penetration and drug efficacy in mice (Patel et al., “Improved transnasal transport and brain uptake of tizanidine HCl-loaded thiolated chitosan nanoparticles for alleviation of pain,” J. Pharm. Sci 101: 690-706 (2012)). Pulmonary administration provides a large surface area and relative ease of access. The mucus barrier, metabolic enzymes in the tracheobronchial region and macrophages in the alveoli are typically the main barriers for drug penetration. Particle size is a major factor determining the diffusion of nanoformulation in the bronchial tree, with particles in the nano-sized region more likely to reach the alveolar region and particles with diameters between 1 and 5 m expected to deposit in the bronchioles (Musante et al., “Factors affecting the deposition of inhaled porous drug particles,” J Pharm Sci 91: 1590-1600 (2002)). A limit to absorption has been shown for larger particles, presumably because of an inability to cross the air-blood barrier. Particles can gradually release the drug, which can consequently penetrate into the blood stream or, alternatively, particles can be phagocytosed by alveolar macrophages (Bailey and Berkland, “Nanoparticle formulations in pulmonary drug delivery,” Med. Res. Rev., 29: 196-212 (2009)).

Certain nanoformulations have a minimal penetration through biological membranes in sites of absorption and for these, i.v. administration can be the preferred route to obtain an efficient distribution in the body (Wacker, “Nanocarriers for intravenous injection—The long hard road to the market,” Int. J. Pharm., 457: 50-62., 2013).

The distribution of nanoformulations can vary widely depending on the delivery system used, the characteristics of the nanoformulation, the variability between individuals, and the rate of drug loss from the nanoformulations. Certain nanoparticles, such as solid drug nanoparticles (SDNs), improve drug absorption, which does not require them to arrive intact in the systemic circulation. Other nanoparticles survive the absorption process, thus altering the distribution and clearance of the contained drug.

Nanoformulations of a certain size and composition can diffuse in tissues through well-characterized processes, such as the enhanced permeability and retention effect, whereas others accumulate in specific cell populations, which allows one to target specific organs. Complex biological barriers can protect organs from exogenous compounds, and the blood-brain barrier (BBB) represents an obstacle for many therapeutic agents. Many different types of cells including endothelial cells, microglia, pericytes and astrocytes are present in the BBB, which exhibits extremely restrictive tight junctions, along with highly active efflux mechanisms, limiting the permeation of most drugs. Transport through the BBB is typically restricted to small lipophilic molecules and nutrients that are carried by specific transporters. One of the most important mechanisms regulating diffusion of nanoformulations into the brain is endocytosis by brain capillary endothelial cells.

Recent studies have correlated particle properties with nanoformulation entry pathways and processing in the human BBB endothelial barrier, indicating that uncoated nanoparticles have limited penetration through the BBB and that surface modification can influence the efficiency and mechanisms of endocytosis (Lee et al., “Targeting rat anti-mouse transferrin receptor monoclonal antibodies through blood-brain barrier in mouse,” J. Pharmacol. Exp. Ther. 292: 1048-1052 (2000)). Accordingly, surface-modified nanoparticles which cross the BBB, and deliver one or more of the compounds described herein, are within the scope of the disclosure.

Macrophages in the liver are a major pool of the total number of macrophages in the body. Kupffer cells in the liver possess numerous receptors for selective phagocytosis of opsonized particles (receptors for complement proteins and for the fragment crystallizable part of IgG). Phagocytosis can provide a mechanism for targeting the macrophages, and providing local delivery (i.e., delivery inside the macrophages) of the compounds described herein (TRUE?).

Nanoparticles linked to polyethylene glycol (PEG) have minimal interactions with receptors, which inhibits phagocytosis by the mononuclear phagocytic system (Bazile et al., “Stealth Me.PEG-PLA nanoparticles avoid uptake by the mononuclear phagocytes system,” J. Pharm. Sci. 84: 493-498 (1995)).

Representative nanoformulations include inorganic nanoparticles, SDNs, SLNs, NEs, liposomes, polymeric nanoparticles and dendrimers. The compounds described herein can be contained inside a nanoformulation, or, as is sometimes the case with inorganic nanoparticles and dendrimers, attached to the surface. Hybrid nanoformulations, which contain elements of more than one nanoformulation class, can also be used.

SDNs are lipid-free nanoparticles, which can improve the oral bioavailability and exposure of poorly water-soluble drugs (Chan, “Nanodrug particles and nanoformulations for drug delivery,” Adv. Drug. Deliv. Rev. 63: 405 (2011)). SDNs include a drug and a stabilizer, and are produced using ‘top-down’ (high pressure homogenization and wet milling) or bottom-up (solvent evaporation and precipitation) approaches.

SLNs consist of a lipid (or lipids) which is solid at room temperature, an emulsifier and water. Lipids utilized include, but are not limited to, triglycerides, partial glycerides, fatty acids, steroids and waxes. SLNs are most suited for delivering highly lipophilic drugs.

Liquid droplets of less than a 1000 nm dispersed in an immiscible liquid are classified as NEs. NEs are used as carriers for both hydrophobic and hydrophilic agents, and can be administered orally, transdermally, intravenously, intranasally, and ocularly. Oral administration can be preferred for chronic therapy, and NEs can effectively enhance oral bioavailability of small molecules, peptides and proteins.

Polymeric nanoparticles are solid particles typically around 200-800 nm in size, which can include synthetic and/or natural polymers, and can optionally be pegylated to minimize phagocytosis. Polymeric nanoparticles can increase the bioavailability of drugs and other substances, compared with traditional formulations. Their clearance depends on several factors, including the choice of polymers (including polymer size, polymer charge and targeting ligands), with positively charged nanoparticles larger than 100 nm being eliminated predominantly via the liver (Alexis et al., Factors affecting the clearance and biodistribution of polymeric nanoparticles. Mol Pharm 5: 505-515 (2008)).

Dendrimers are tree-like, nanostructured polymers which are commonly 10-20 nm in diameter.

Liposomes are spherical vesicles which include a phospholipid bilayer. A variety of lipids can be utilized, allowing for a degree of control in degradation level. In addition to oral dosing, liposomes can be administered in many ways, including intravenously (McCaskill et al., 2013), transdermally (Pierre and Dos Santos Miranda Costa, 2011), intravitreally (Honda et al., 2013) and through the lung (Chattopadhyay, 2013). Liposomes can be combined with synthetic polymers to form lipid-polymer hybrid nanoparticles, extending their ability to target specific sites in the body. The clearance rate of liposome-encased drugs is determined by both drug release and destruction of liposomes (uptake of liposomes by phagocyte immune cells, aggregation, pH-sensitive breakdown, etc.) (Ishida et al., “Liposome clearance,” Biosci Rep 22: 197-224 (2002)).

One of more of these nanoparticulate formulations can be used to deliver the active agents described herein to the macrophages, across the blood brain barrier, and other locations as appropriate.

### Controlled Release Formulations

In a preferred embodiment, the active compounds are prepared with carriers that will protect the compound against rapid elimination from the body, such as a controlled release formulation, including but not limited to implants and microencapsulated delivery systems. Biodegradable, biocompatible polymers can be used, such as ethylene vinyl acetate, polyanhydrides, polyglycolic acid, collagen, polyorthoesters and polylactic acid. For example, enterically coated compounds can be used to protect cleavage by stomach acid. Methods for preparation of such formulations will be apparent to those skilled in the art. Suitable materials can also be obtained commercially.

Liposomal suspensions (including but not limited to liposomes targeted to infected cells with monoclonal antibodies to viral antigens) are also preferred as pharmaceutically acceptable carriers. These can be prepared according to methods known to those skilled in the art, for example, as described in U.S. Pat. No. 4,522,811 (incorporated by reference). For example, liposome formulations can be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

The terms used in describing the invention are commonly used and known to those skilled in the art. As used herein, the following abbreviations have the indicated meanings:
DMSO dimethylsulfoxide
DCM dichloromethane
DMAP 4-dimethylaminopyridine
EtOAc (AcOEt) ethyl acetate
HOAc Acetic acid
h hour
hex hexane
DIPEA Diisopropylethylamine
Liq. Liquid
LCMS Liquid chromatography mass spectrometry
TLC thin layer chromatography
M molar
MeOH Methanol
EtOH Ethanol
iPrOH Isopropyl alcohol
nBuOH n-Butyl alcohol
pTsOH p-Toluene sulfonic acid
TMSCN Trimethylsilylcyanide
TMSCI Trimethylsilylchloride
TMSOTf Trimethylsilyltriflate
Et₃N Triethylamine
nBuLi n-Butyl lithium
min minute
rt or RT room temperature
TBAF Tetrabutylamrnonium fluoride
THF tetrahydrofuran

### IX. General Methods for Preparing Active Compounds

Methods for the facile preparation of active compounds are known in the art and result from the selective combination known methods. The compounds disclosed herein can be prepared as described in detail below, or by other methods known to those skilled in the art. It will be understood by one of ordinary skill in the art that variations of detail can be made without departing from the spirit and in no way limiting the scope of the present invention.

Some compounds within certain of the general formulas described herein are commercially available. For some compounds, the syntheses described herein are exemplary and can be used as a starting point to prepare additional compounds of the formulas described herein. These compounds can be prepared in various ways, including those synthetic schemes shown and described herein. Those skilled in the art will be able to recognize modifications of the disclosed syntheses and to devise routes based on the disclosures herein; all such modifications and alternate routes are within the scope of the claims.

The various reaction schemes are summarized below.
Scheme 1 is a synthetic approach to nucleosides 3. (Base and other variables listed in the Scheme are as defined in active compound section)
Scheme 2 is an alternate synthetic approach to nucleosides 3. (Base and other variables listed in the Scheme are as defined in active compound section)

Compounds of Formula A can be prepared by first preparing nucleosides 1, which in turn can be accomplished by one of ordinary skill in the art, using methods outlined in: (a) Rajagopalan, P.; Boudinot, F. D; Chu, C. K.; Tennant, B. C.; Baldwin, B. H.; Antiviral Nucleosides: Chiral Synthesis and Chemotheraphy: Chu, C. K.; Eds. Elsevier: 2003. b) Recent Advances in Nucleosides: Chemistry and Chemotherapy: Chu, C. K.; Eds. Elsevier: 2002. c) Frontiers in Nucleosides & Nucleic Acids, 2004, Eds. R. F. Schinazi & D. C. Liotta, HIL Press, Tucker, GA, USA, pp: 319-37 d) Handbook of Nucleoside Synthesis: Vorbruggen H. & Ruh-Pohlenz C. John Wiley & sons 2001), and by general Schemes 1-2. Specifically, nucleosides 3 can be prepared by coupling sugar 1 with a protected, silylated or free nucleoside base in the presence of Lewis acid such as TMSOTf Deprotection of the 3′- and 5′-hydroxyls gives nucleoside 3.

Analogous compounds of Formula B can be prepared using compounds like Compound 1, but with a fluorine rather than OPr at the 2′-position. Representative synthetic methods are described, for example, in U.S. Pat. No. 8,716,262.

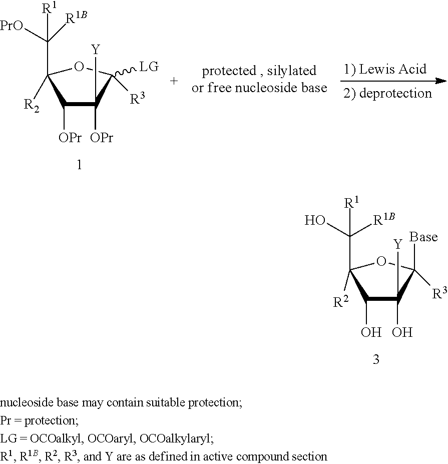

Similarly, compounds like Compound 1, but with a Y substituent at the 2′-position and/or an R substituent at the 3′-position, can be used to prepare nucleosides similar to Compound 3, but with Y or R substitution at the 2′- and/or 3′-positions, respectively.

Also, analogous compounds where the oxygen in the sugar ring is replaced with one of the other variables defined by R⁵ can also be prepared.

### Scheme 1 a Synthetic Approach to Nucleosides 3. (Base are as Defined in Active Compound Section)

In the schemes described herein, if a nucleoside base includes functional groups that might interfere with, or be decomposed or otherwise converted during the coupling steps, such functional groups can be protected using suitable protecting groups. After the coupling step, protected functional groups, if any, can be deprotected.

Alternatively, nucleosides 3 can be prepared from 1′-halo, 1′-sulfonate or 1′-hydroxy compounds 2. For the case of 1′-halo or 1′-sulfonate a protected or free nucleoside base in the presence of a base such as triethyl amine or sodium hydride followed by deprotection would give nucleosides 3. For the case of 1′-hydroxy a protected or free nucleoside base in the presence of a Mitsunobu coupling agent such as diisopropyl azodicarboxylate followed by deprotection would give nucleosides 3.

Analogous compounds of Formula B can be prepared using compounds like Compound 1, but with a fluorine rather than OPr at the 2′-position. Representative synthetic methods are described, for example, in U.S. Pat. No. 8,716,262.

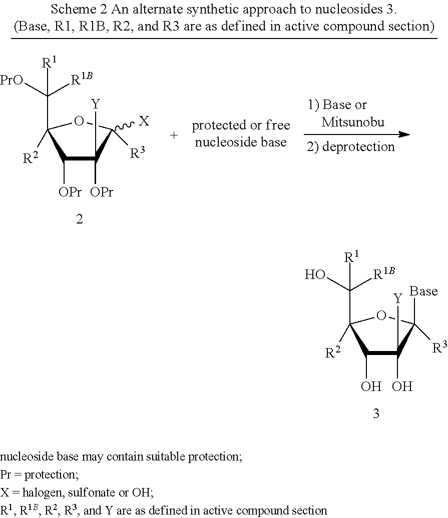

Similarly, compounds like Compound 2, but with a Y substituent at the 2′-position and/or an R substituent at the 3′-position, can be used to prepare nucleosides similar to Compound 3, but with Y or R substitution at the 2′- and/or 3′-positions, respectively.

Also, analogous compounds where the oxygen in the sugar ring is replaced with one of the other variables defined by R⁵ can also be prepared.

In the schemes described herein, if a nucleoside base includes functional groups that might interfere with, or be decomposed or otherwise converted during the reaction steps, such functional groups can be protected using suitable protecting groups that can be removed. Protected functional groups, if any, can be deprotected later on.

In the case of C-nucleosides prepared from bases:

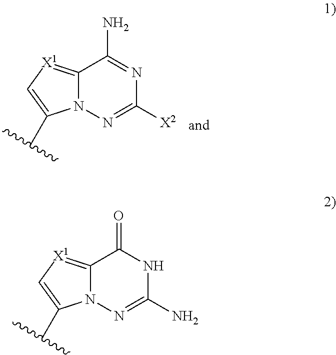

methods outlined in WO09132123, WO09132135, WO2011150288 and WO2011035250 can be used.

In the case of C-nucleosides prepared from other bases, methods outlined in Temburnikar K, Seley-Radtke K L. Recent advances in synthetic approaches for medicinal chemistry of C-nucleosides. Beilstein J Org Chem. 2018; 14:772-785 can be used.

In the case of carbocyclic nucleosides, methods outlined in the following references can be used:
Advances in the enantioselective synthesis of carbocyclic nucleosides, Chem. Soc. Rev., 2013, 42, 5056
The latest progress in the synthesis of carbocyclic nucleosides”. Nucleosides, Nucleotides & Nucleic Acids. 2000, 19 (3): 651-690
New progresses in the enantioselective synthesis and biological properties of carbocyclic nucleosides”. Mini Reviews in Medicinal Chemistry 2003, 3(2): 95-114.
Chemical synthesis of carbocyclic analogues of nucleosides”. Chemical Synthesis of Nucleoside Analogues. Hoboken: John Wiley & Sons. 2003 pp. 535-604

Dioxolane nucleoside analags can be prepared by adapting the chemistry outlined in J. Org. Chem. 1995, 60, 6, 1546-1553.

### Incorporation of Deuterium:

It is expected that single or multiple replacement of hydrogen with deuterium (carbon-hydrogen bonds to carbon-deuterium bond) at site(s) of metabolism in the sugar portion of a nucleoside antiviral agent will slow down the rate of metabolism. This can provide a relatively longer half-life, and slower clearance from the body. The slow metabolism of a therapeutic nucleoside is expected to add extra advantage to a therapeutic candidate, while other physical or biochemical properties are not affected. Intracellular hydrolysis or deuterium exchanges my result in liberation of deuterium oxide (D20).

Methods for incorporating deuterium into amino acids, phenol, sugars, and bases, are well known to those of skill in the art. Representative methods are disclosed in U.S. Pat. No. 9,045,521.

A large variety of enzymatic and chemical methods have been developed for deuterium incorporation at both the sugar and nucleoside stages to provide high levels of deuterium incorporation (D/H ratio). The enzymatic method of deuterium exchange generally has low levels of incorporation. Enzymatic incorporation has further complications due to cumbersome isolation techniques which are required for isolation of deuterated mononucleotide blocks. Schmidt et al., Ann. Chem. 1974, 1856; Schmidt et al., Chem. Ber., 1968, 101, 590, describes synthesis of 5′,5′-²H₂-adenosine which was prepared from 2′,3′-O-isopropylideneadenosine-5′-carboxylic acid or from methyl-2,3-isopropylidene-beta-D-ribofuranosiduronic acid, Dupre, M. and Gaudemer, A., Tetrahedron Lett. 1978, 2783. Kintanar, et al., Am. Chem. Soc. 1998, 110, 6367 reported that diastereoisomeric mixtures of 5′-deuterioadenosine and 5′(R/S)-deuteratedthymidine can be obtained with reduction of the appropriate 5′-aldehydes using sodium borodeuteride or lithium aluminum deuteride (98 atom % ²H incorporation). Berger et al., Nucleoside & Nucleotides 1987, 6, 395 described the conversion of the 5′-aldehyde derivative of 2′deoxyguanosine to 5′ or 4′-deuterio-2′-deoxyguanosine by heating the aldehyde in ²H₂O/pyridine mixture (1:1) followed by reduction of the aldehyde with NaBD₄.

Ajmera et al., Labelled Compd. 1986, 23, 963 described procedures to obtain 4′-deuterium labeled uridine and thymidine (98 atom % ²H). Sinhababu, et al., J. Am. Chem. Soc. 1985, 107, 7628) demonstrated deuterium incorporation at the C3′ (97 atom % ²H) of adenosine during sugar synthesis upon stereoselective reduction of 1,2:5,6-di-O-isopropylidene-β-D-hexofuranos-3-ulose to 1,2:5,6-di-O-isopropylidene-3-deuterio-β-D-ribohexofuranose using sodium borodeuteride and subsequently proceeding further to the nucleoside synthesis. Robins, et al., Org. Chem. 1990, 55, 410 reported synthesis of more than 95% atom ²H incorporation at C3′ of adenosine with virtually complete stereoselectivity upon reduction of the 2′-O-tert-butyldimethylsilyl (TBDMS) 3-ketonucleoside by sodium borodeuteride in acetic acid. David, S. and Eustache, J., Carbohyd. Res. 1971, 16, 46 and David, S. and Eustache, J., Carbohyd. Res. 1971, 20, 319 described syntheses of 2′-deoxy-2′(S)-deuterio-uridine and cytidine. The synthesis was carried out by the use of 1-methyl-2-deoxy-2′-(S)-deuterio ribofuranoside.

Radatus, et al., J. Am. Chem. Soc. 1971, 93, 3086 described chemical procedures for synthesizing 2′-monodeuterated (R or S)-2′-deoxycytidines. These structures were synthesized from selective 2-monodeuterated-2-deoxy-D-riboses, which were obtained upon stereospecific reduction of a 2,3-dehydro-hexopyranose with lithium aluminum deuteride and oxidation of the resulting glycal. Wong et al. J. Am. Chem. Soc. 1978, 100, 3548 reported obtaining deoxy-1-deuterio-D-erythro-pentose, 2-deoxy-2(S)-deuterio-D-erythro-pentose and 2-deoxy-1,2(S)-dideuterio-D-erythro-pentose from D-arabinose by a reaction sequence involving the formation and LiAlD4 reduction of ketene dithioacetal derivatives.

Pathak et al. J., Tetrahedron 1986, 42, 5427) reported stereospecific synthesis of all eight 2′ or 2′-deuterio-2′-deoxynucleosides by reductive opening of appropriate methyl 2,3-anhydro-beta-D-ribo or beta-D-lyxofuranosides with LiAlD4. Wu et al. J. Tetrahedron 1987, 43, 2355 described the synthesis of all 2′,2″-dideuterio-2′-deoxynucleosides, for both deoxy and ribonucleosides, starting with oxidation of C2′ of sugar and subsequent reduction with NaBD₄ or LiAlD₄ followed by deoxygenation by tributyltin deuteride. Roy et al. J. Am. Chem. Soc. 1986, 108, 1675, reported 2′,2′-dideuterio-2′-deoxyguanosine and thymidine can be prepared from 2-deoxyribose 5-phosphate using 2-deoxyribose 5-phosphate aldolase enzyme in ²H₂O achieving some 90 atom % deuteration. Similarly, the synthesis of 4′,5′,5′-²H₃-guanosine can be carried out.

Therefore, it is clear that each position of the sugar residue can be selectively labeled.

A useful alternative method of stereospecific deuteration was developed to synthesize polydeuterated sugars. This method employed exchange of hydrogen with deuterium at the hydroxyl bearing carbon (i.e. methylene and methine protons of hydroxyl bearing carbon) using deuterated Raney nickel catalyst in ²H₂O.

Various techniques are available to synthesize fully deuterated deoxy and ribonucleosides. Thus, in one method, exchange reaction of deuterated Raney nickel-²H₂O with sugars, a number of deuterated nucleosides specifically labeled at 2′, 3′ and 4′ positions were prepared. The procedure consisted of deuteration at 2′, 3′ and 4′ positions of methyl beta-D-arabinopyranoside by Raney nickel-²H₂O exchange reaction followed by reductive elimination of ‘2-hydroxyl group by tributyltin deuteride to give methyl beta-D-2′,2′,3′,4′-²H₄-2-deoxyribopyranoside, which was converted to methyl beta-D-2′,2′,3′,4′-²H₄-2′-deoxyribofuranoside and glycosylated to give various 2′,2′,3′,4′-²H₄-nucleosides (>97 atom % ²H incorporation for H3′ & H4′.

The synthesis of deuterated phenols is described, for example, in Hoyer, H. (1950), Synthese des pan-Deutero-o-nitro-phenols. Chem. Ber., 83: 131-136. This chemistry can be adapted to prepare substituted phenols with deuterium labels. Deuterated phenols, and substituted analogs thereof, can be used, for example, to prepare phenoxy groups in phosphoramidate prodrugs.

The synthesis of deuterated amino acids is described, for example, in Matthews et al., Biochimica et Biophysica Acta (BBA)—General Subjects, Volume 497, Issue 1, 29 Mar. 1977, Pages 1-13. These and similar techniques can be used to prepare deuterated amino acids, which can be used to prepare phosphoramidate prodrugs of the nucleosides described herein.

One method for synthesizing a deuterated analog of the compounds described herein involves synthesizing a deuterated ribofuranoside with a 4′-alkynyl substitution; and attaching a nucleobase to the deuterated ribofuranoside to form a deuterated nucleoside. A prodrug, such as a phosphoramidate prodrug, can be formed by modifying the 5′-OH group on the nucleoside. Where a deuterated phenol and/or deuterated amino acid is used, one can prepare a deuterated phosphoramidate prodrug.

Another method involves synthesizing a ribofuranoside with 4′-alkynyl substitution, and attaching a deuterated nucleobase to form a deuterated nucleoside. This method can optionally be performed using a deuterated furanoside to provide additional deuteration. As with the method described above, the nucleoside can be converted into a prodrug form, which prodrug form can optionally include additional deuteration.

A third method involves synthesizing a ribofuranoside with 4′-alkynyl substitution, attaching a nucleobase to form a nucleoside, and converting the nucleoside to a phosphoramidate prodrug using one or both of a deuterated amino acid or phenol analog in the phosphoramidate synthesis.

Accordingly, using the techniques described above, one can provide one or more deuterium atoms in the sugar, base, and/or prodrug portion of the nucleoside compounds described herein.

### SPECIFIC EXAMPLES

Specific representative compounds were prepared as per the following examples and reaction sequences; the examples and the diagrams depicting the reaction sequences are offered by way of illustration, to aid in the understanding of the invention and should not be construed to limit in any way the invention set forth in the claims which follow thereafter. The present compounds can also be used as intermediates in subsequent examples to produce additional compounds as described herein. No attempt has necessarily been made to optimize the yields obtained in any of the reactions. One skilled in the art would know how to increase such yields through routine variations in reaction times, temperatures, solvents and/or reagents.

Anhydrous solvents were purchased from Aldrich Chemical Company, Inc. (Milwaukee, WI) and EMD Chemicals Inc. (Gibbstown, NJ). Reagents were purchased from commercial sources. Unless noted otherwise, the materials used in the examples were obtained from readily available commercial suppliers or synthesized by standard methods known to one skilled in the art of chemical synthesis. Melting points (mp) were determined on an Electrothermal digit melting point apparatus and are uncorrected. ¹H and ¹³C NMR spectra were taken on a Varian Unity Plus 400 spectrometer at room temperature and reported in ppm downfield from internal tetramethylsilane. Deuterium exchange, decoupling experiments or 2D-COSY were performed to confirm proton assignments. Signal multiplicities are represented by s (singlet), d (doublet), dd (doublet of doublets), t (triplet), q (quadruplet), br (broad), bs (broad singlet), m (multiplet). All J-values are in Hz. Mass spectra were determined on a Micromass Platform LC spectrometer using electrospray techniques. Elemental analyses were performed by Atlantic Microlab Inc. (Norcross, GA). Analytic TLC was performed on Whatman LK6F silica gel plates, and preparative TLC on Whatman PK5F silica gel plates. Column chromatography was carried out on Silica Gel or via reverse-phase high performance liquid chromatography.

### Example 1

The techniques shown below can be used to prepare other compounds described herein

### Experimental

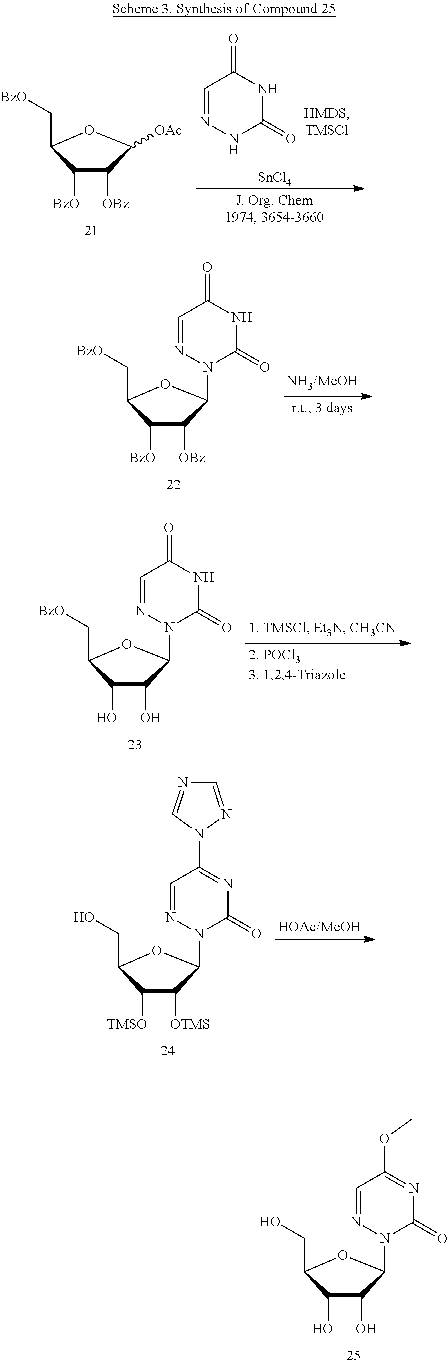

### 2-((2R,3R,4S,5R)-3,4-Dihydroxy-5-(hydroxymethyl)tetrahydrofuran-2-yl)-1,2,4-triazine-3,5(2H,4H)-dione (23)

A mixture of compound 22 (10.5 g, prepared by following the chemistry described in J. Org. Chem. 1974, 3654-3660) in saturated NH₃/MeOH (250 mL) was stirred at room temperature for 3 days. After removal of the volatiles under reduced pressure, the residue was purified by flash chromatography (0-20% MeOH in dichloromethane) to give 23 (3.5 g, 76%). ¹H NMR (CD₃OD): 7.44 (s, 1H), 6.08 (d, J=3.2 Hz, 1H), 4.42 (dd, J=5.2 Hz, J=3.2 Hz, 1H), 4.24 (t, J=5.6 Hz, 1H), 3.96 (m, 1H), 3.73 (dd, J=12.0 Hz, J=3.6 Hz, 1H), 3.60 (dd, J=12.0 Hz, J=5.6 Hz, 1H); ¹³C NMR (CD₃OD): 158.27, 149.97, 137.36, 91.71, 85.87, 74.42, 71.91, 63.43; LCMS: 246 (M+1)⁺.

### 2-((2R,3R,4S,5R)-3,4-Dihydroxy-5-(hydroxymethyl)tetrahydrofuran-2-yl)-5-methoxy-1,2,4-triazin-3(2H)-one (25)

To a stirred suspension of 23 (123 mg, 0.5 mmol, 1 eq) in acetonitrile (5 mL), triethylamine (1.05 mL, 7.5 mmol, 15 eq) and chlorotrimethylsilane (0.32 mL, 2.5 mmol, 5 eq) were added dropwise at 0° C. The cooling bath was removed and the reaction mixture was stirred at room temperature for 2 hours. The reaction mixture was then cooled down to 0° C. and POCl₃ (145 uL) was added dropwise. The reaction mixture was stirred for 15 min and 1,2,4-triazole (345 mg, 5 mmol, 10 eq) was added. The reaction mixture was stirred at room temperature overnight, and then poured into a pH 7.4 buffer solution (20 mL). The mixture was extracted with DCM (3×30 mL). The combined organic phases were dried over sodium sulfate. After the volatiles were removed under reduced pressure, the residue was dissolved in MeOH/HOAc (4:1, 5 mL) and stirred overnight. After removal of the voaltils, the residue was purified by column using 0-10% methanol in DCM to give product 3 (70.6 mg, 54%). LCMS: 282 (M+Na+). ¹H NMR (CD₃OD): 7.80 (s, 1H), 6.20 (d, J=3.2 Hz, 1H), 4.43 (dd, J=5.2 Hz, J=3.2 Hz, 1H), 4.28 (t, J=5.6 Hz, 1H), 4.03-4.00 (m, 1H), 4.01 (s, 3H), 3.75 (dd, J=8.0 Hz, J=3.6 Hz, 1H), 3.62 (dd, J=12.4 Hz, J=5.6 Hz, 1H); ¹³C NMR (CD₃OD): 166.70, 155.93, 130.25, 93.29, 86.11, 74.83, 71.93, 63.43, 55.68.

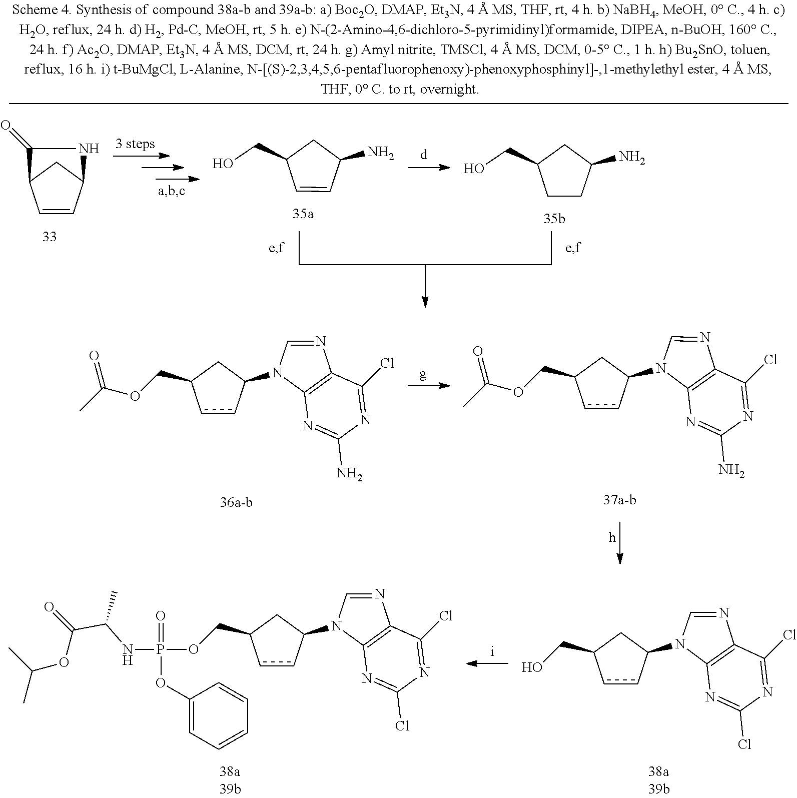

### ((1S,4R)-4-Aminocyclopent-2-en-1-yl)methanol (35a) was prepared according to the procedures reported in J. Am. Chem. Soc. 2005, 127, 24, 8846-8855

A mixture of Vince lactam 33 (1 eq.), di-t-butyl dicarbonate (1.2 eq.), DMAP (0.1 eq.) and Et₃N (1.2 eq.) in THF (0.5 M) was stirred at room temperature for 4 hours and then evaporated. The residue was dissolved in AcOEt, washed with 1M HCl, then with a solution of 5% NaHCO₃ and brine, dried over MgSO₄ filtered and concentrated under vacuo. Crude product was purified flash chromatography on silica gel (Hexanes/AcOEt—1:0 to 85:25) to afford pure NBoc intermediate as a white solid (92%). This compound was dissolved in methanol (0.3 M) and cool down to 0° C. NaBH₄ (4 eq.) was added in 6 portions over 1 hour and the reaction was stirred at 0° C. for 30 min and then 4 hours at room temperature. Volatiles were evaporated and the residue was partitioned between water and AcOEt (2:3). The aqueous phase was extracted with AcOEt. Combined organic layers were washed with brine, dried over MgSO₄, filtered and concentrated under vacuo. The resulting white solid was refluxed in water (0.25 M) for 24 hours. Water was removed under vacuo to afford the desired compound (4) as a brown oil.

### ((1R,3S)-3-Aminocyclopentyl)methanol (35b) was prepared according to the procedures reported in J. Am. Chem. Soc. 2005, 127, 24, 8846-8855

A mixture of 35a (1 eq.) and 10% Pd—C (0.04 eq.) in MeOH (0.115 M) was stirred under atmospheric pressure of H₂ at room temperature for 4 hours. The Pd—C was filtered off on a Celite pad, washed with MeOH, and the combined filtrate were evaporated to afford 35b as a slightly brown oil (quantitative yield).

### ((1S,4R)-4-(2-Amino-6-chloro-9H-purin-9-yl)cyclopent-2-en-1-yl)methyl acetate (36a) and ((1R,3S)-3-(2-amino-6-chloro-9H-purin-9-yl)cyclopentyl)methyl acetate (36b)

In a sealed vessel, to a solution of compound 35a or 35b (1 eq.) in n-BuOH (0.3 M), was added DIPEA (4 eq.) and N-(2-Amino-4,6-dichloro-5-pyrimidinyl)formamide (1.5 eq.). The mixture was heated at 130° C. for 24 hours. Volatiles were evaporated and the crude product was purified silica gel flash chromatography (DCM/MeOH—1:0 to 95:5). The resulting compound was suspended in DCM (0.1 M) and DMAP (0.2 eq.), Et₃N (2 eq.) and Ac20 (1.1 eq.) were added at room temperature. The clear solution was stirred for 24 hours at room temperature, then the volatiles were removed under vacuum. The residue was dissolved in a saturated solution of NaHCO₃, extracted thrice with AcOEt. The combined organic layers were washed with brine, dried over MgSO₄, filtered and concentrated under vacuo. The residue was purified using silica gel flash chromatography (DCM/MeOH—1:0 to 95:5) to afford compounds (6a,b).

(36a). (48%, over 2 steps). ¹H NMR (400 MHz, Acetone-d6) δ 8.00 (d, J=1.3 Hz, 1H, H8), 6.26-6.19 (m, 3H, NH₂, H2′), 6.06 (dq, J=5.6, 1.9 Hz, 1H, H3′), 5.64 (dddd, J=9.0, 5.9, 3.5, 1.8 Hz, 1H, H1′), 4.21 (dd, J=6.1, 1.2 Hz, 2H, H5′), 3.24 (ddt, J=8.1, 5.9, 3.4 Hz, 1H, H4′), 2.93-2.77 (m, 1H, H6′a), 2.05 (s, 3H, C—CH₃), 1.83 (dtd, J=13.7, 6.0, 1.2 Hz, 1H, H6′b). ¹³C NMR (101 MHz, Acetone-d6) δ 171.0 (C═O), 160.6 (Cquat), 154.8 (Cquat), 151.0 (Cquat), 141.6 (C8), 138.1 (C2′), 131.0 (C3′), 125.7 (Cquat), 67.0 (C5′), 60.4 (C1′), 45.3 (C4′), 35.0 (C6′), 20.7 (CO—CH₃). HRMS-ESI (m/z) [M+H]+ calcd for C₁₃H₁₄ClN₅O₂: 307.0836, found: 307.0902.

(36b). (50%, over 2 steps). ¹H NMR (400 MHz, Acetone-d6) δ 8.13 (s, 1H, H8), 6.20 (bs, 2H, NH₂), 4.88 (dq, J=9.8, 8.1 Hz, 1H, H1′), 4.22-4.03 (m, 2H, H5′), 2.58-2.40 (m, 2H, H4′, H2′a), 2.36-2.10 (m, 2H, H3′), 2.05 (s, 3H, C—CH₃), 2.08-1.89 (m, 2H, H2′b, H6′a), 1.88-1.75 (m, 1H, H6′b). ¹³C NMR (101 MHz, Acetone-d6) δ 171.1 (C═O), 160.5 (Cquat), 155.0 (Cquat), 151.0 (Cquat), 142.2 (C8), 125.9 (Cquat), 68.1 (C5′), 56.4 (C1′), 37.9 (C4′), 36.0 (C2′), 31.5 (C3′), 27.6 (C6′), 20.7 (CO—CH₃). HRMS-ESI (m/z) [M+H]+ calcd for C₁₃H₁₆ClN₅O₂: 309.0993, found: 309.1063.

### ((1S,4R)-4-(2,6-dichloro-9H-purin-9-yl)cyclopent-2-en-1-yl)methyl acetate (37a) and ((1R,3S)-3-(2,6-dichloro-9H-purin-9-yl)cyclopentyl)methyl acetate (37b)

A mixture of amyl nitrite (6 eq.) in DCM (0.47 M) was cool down to 0° C. TMSCI (3 eq.) was added dropwise followed by a solution of (36a,b) (1 eq.) in DCM (0.47 M). The mixture was stirred between 0 and 5° C. for 1 hour and then quenched with a saturated solution of Na₂SO₃. The aqueous layer was extracted thrice with DCM. Combined organic layers were washed with saturated NaHCO₃ and brine, dried over MgSO₄, filtered and concentrated under vacuo. The residue purified by flash chromatography (Hex/AcOEt—2:8 to 8:2) to afford compounds (7a,b).

(37a). (74%). ¹H NMR (400 MHz, Acetone-d6) δ 8.51 (s, 1H, H8), 6.27 (dt, J=5.7, 2.1 Hz, 1H, H2′), 6.10 (dt, J=5.6, 2.2 Hz, 1H, H3′), 5.82 (ddq, J=9.9, 6.0, 2.1 Hz, 1H, H1′), 4.16 (dq, J=11.0, 5.8, 5.4 Hz, 2H, H5′), 3.26 (tddd, J=8.1, 5.9, 4.1, 2.2 Hz, 1H, H4′), 2.94 (dt, J=14.0, 8.7 Hz, 1H, H6′a), 2.00 (s, 3H, C—CH₃), 1.90 (dt, J=14.1, 5.9 Hz, 1H, H6′b). ¹³C NMR (101 MHz, Acetone-d6) δ 171.0 (C═O), 154.3 (Cquat), 152.4 (Cquat), 151.1 (Cquat), 140.6 (C8), 139.1 (C2′), 132.2 (Cquat), 130.2 (C3′), 66.9 (C5′), 61.6 (C1′), 45.5 (C4′), 35.1 (C6′), 20.7 (CO—CH₃). HRMS-ESI (m/z) [M+H]+ calcd for C₁₃H₁₂Cl₂N₄O₂: 326.0337, found: 326.0408.

(37b). (76%). ¹H NMR (400 MHz, Acetone-d6) δ 8.70 (s, 1H, H8), 5.22-5.00 (m, 1H, H1′), 4.23-4.07 (m, 2H, H5′), 2.65-2.47 (m, 2H, H4′, H2′a), 2.40 (dddd, J=14.0, 7.9, 6.2, 1.4 Hz, 1H, H3′a), 2.35-2.23 (m, 1H, H3′b), 2.05 (s, 3H, C—CH₃), 2.09-1.94 (m, 2H, H2′b, H6′a), 1.93-1.78 (m, 1H, H6′b). ¹³C NMR (101 MHz, Acetone-d6) δ 171.0 (C═O), 154.5 (Cquat), 152.3 (Cquat), 151.2 (Cquat), 147.0 (C8), 132.2 (Cquat), 67.9 (C5′), 57.3 (C1′), 37.9 (C4′), 36.3 (C2′), 31.7 (C3′), 27.5 (C6′), 20.7 (CO—CH₃). HRMS-ESI (m/z) [M+H]+ calcd for C₁₃H₁₄Cl₂N₄O₂: 328.0494, found: 328.0565. Rf: 0.4 (DCM/MeOH 95:5).

### ((1S,4R)-4-(2,6-dichloro-9H-purin-9-yl)cyclopent-2-en-1-yl)methanol (38a) and ((1R,3S)-3-(2,6-dichloro-9H-purin-9-yl)cyclopentyl)methanol (38b)

A mixture of 37a,b (1 eq.) and Bu₂SnO (3 eq.) in toluene (0.0075 M) was refluxed for 16-18 hours and then the solvent was removed in vacuo. The residue was purified by flash chromatography (Hex/AcOEt—1:9 to 0:1. to afford product 8a,b.

(38a). (70%). ¹H NMR (400 MHz, Methanol-d4) δ 8.57 (s, 1H, H8), 6.26 (dt, J=5.7, 2.1 Hz, 1H, H2′), 5.97 (dt, J=5.6, 2.2 Hz, 1H, H3′), 5.77 (ddq, J=9.2, 5.6, 2.0 Hz, 1H, H1′), 3.69-3.55 (m, 2H, H5′), 3.11-2.99 (m, 1H, H4′), 2.85 (dt, J=14.1, 8.9 Hz, 1H, H6′a), 1.81 (dt, J=14.1, 5.4 Hz, 1H, H6′b). ¹³C NMR (101 MHz, Methanol-d4) δ 154.34 (Cquat), 153.55 (Cquat), 151.66 (Cquat), 147.55 (C8), 140.99 (C2′), 131.89 (Cquat), 129.71 (C3′), 65.06 (C5′), 62.33 (C1′), 49.28 (C4′), 35.10 (C6′). HRMS-ESI (m/z) [M+H]+ calcd for C₁₁H₁₀Cl₂N₄O: 284.0232, found: 284.0304.

(38b). (42%). ¹H NMR (400 MHz, Methanol-d4) δ 9.11 (s, 1H, H8), 5.44 (dq, J=9.6, 7.9 Hz, 1H, H1′), 4.07 (dd, J=6.3, 4.4 Hz, 2H, H5′), 2.97-2.87 (d, J=12.6 Hz, 1H, H2′a), 2.83-2.71 (m, 2H, H4′, H3′a), 2.68-2.53 (m, 1H, H3′b), 2.47-2.12 (m, 3H, H2′b, H6′a, H6′b). ¹³C NMR (101 MHz, Methanol-d4) δ 154.6 (Cquat), 153.4 (Cquat), 151.7 (Cquat), 147.7 (C8), 132.0 (Cquat), 66.4 (C5′), 58.3 (C1′), 41.5 (C4′), 36.4 (C2′), 32.2 (C3′), 27.6 (C6′). HRMS-ESI (m/z) [M+H]+ calcd for C₁₁H₁₂Cl₂N₄O: 286.0388, found: 286.0460.

### Isopropyl ((((1S,4R)-4-(2,6-dichloro-9H-purin-9-yl)cyclopent-2-en-1-yl)methoxy)(phenoxy)phosphoryl)-L-alaninate (39a) and isopropyl ((((1R,3S)-3-(2,6-dichloro-9H-purin-9-yl)cyclopentyl)methoxy)(phenoxy)phosphoryl)-L-alaninate (39b)

Under inert atmosphere, compound 38a,b (1 eq.) was dissolved into anhydrous THE (0.26 M), with 4 Å MS. The reaction mixture was cooled down to 0° C. and t-BuMgCl (1.7 M in THF, 3.1 eq.) was added dropwise. The reaction was stirred for 30 min at 0° C. and then stirred for 30 min at room temperature. (S)-2-[-(S)-(2,3,4,5,6-Pentafluoro-phenoxy)-phenoxy-phos-phorylamino] propionic acid isopropyl ester (2.5 eq.) dissolved in THE (0.5 M) was added dropwise to the previous solution at 0° C. After 30 min at that temperature, the reaction was warmed up to room temperature stirred overnight. Volatiles were then removed under vaccum and the residue was diluted in a solution of saturated NaHCO₃. The aqueous layer was extracted 3 times with AcOEt. The combined organic layers were washed with brine, dried over MgSO₄, filtered and concentrated under vacuo. The residue was purified by flash chromatography (Hex/AcOEt—1:1 to 0:1) to afford compound 39a,b.

(39a). (31%). ¹H NMR (400 MHz, Acetone-d6) δ (8.50 (s, 1H, H8), 7.33 (dd, J=8.7, 7.1 Hz, 2H, Harom), 7.29-7.20 (m, 2H, Harom), 7.14 (tq, J=7.7, 1.1 Hz, 1H, Harom), 6.29-6.18 (m, 1H, H2′), 6.09 (dt, J=5.7, 2.2 Hz, 1H, H3′), 5.81 (ddq, J=9.9, 6.0, 2.1 Hz, 1H, H1′), 4.92 (p, J=6.2 Hz, 1H, O—CH), 4.84-4.74 (m, 1H, NH), 4.23-4.11 (m, 2H, H5′), 4.01-3.85 (m, 1H, N—CH), 3.34-3.24 (m, 1H, H4′), 2.91 (dt, J=14.1, 8.8 Hz, 1H, H6′a), 2.80 (t, J=1.1 Hz, 1H, NH), 1.99-1.80 (m, 1H, H6′b), 1.32 (dd, J=7.1, 0.9 Hz, 3H, CH—CH₃), 1.19 (dd, J=6.2, 1.7 Hz, 6H, 2×CH—CH₃). ¹³C NMR (101 MHz, Acetone-d6) δ 173.5 (C═O), 154.3 (Cquat), 152.4 (Cquat), 152.2 Cquat), 151.1 (Cquat), 146.7 (C8), 138.8 (C2′), 132.1 (Cquat), 130.5 (C3′), 130.3 (2×Carom), 125.2 (Carom), 121.1 (2×Carom), 69.5-68.3 (m, C₅′, O—CH), 61.5 (C1′), 51.2 (NH—CH), 46.8 (C4′), 34.71 (C6′), 21.9 (2×C—CH₃), 21.8 (C—CH₃). ³¹P NMR (162 MHz, Acetone-d6) δ 2.78. HRMS-ESI (m/z) [M+H]+ calcd for C₂₃H₂₆Cl₂N₅O₅P: 553.1049, found: 553.1124.

(39b). (48%). ¹H NMR (400 MHz, Acetone-d6) δ 8.63 (s, 1H, H8), 7.34 (dd, J=8.7, 7.0 Hz, 2H, Harom), 7.33-7.23 (m, 2H, Harom), 7.20-7.10 (m, 1H, Harom), 5.05 (d, J=8.1 Hz, 1H, H1′), 4.98-4.90 (m, 1H, O—CH), 4.92-4.77 (m, 1H, NH), 4.14 (qt, J=10.2, 6.4 Hz, 2H, H5′), 4.01-3.87 (m, 1H, NH—CH), 2.64-2.42 (m, 2H, H4′, H2′a), 2.36 (dtd, J=13.4, 7.8, 5.5 Hz, 1H, H3′a), 2.20 (dtd, J=12.9, 9.0, 7.4 Hz, 1H, H3′b), 2.00-1.93 (m, 2H, H2′b, H6′a), 1.92-1.79 (m, 1H, H6′a), 1.39-1.26 (m, 3H, CH—CH₃), 1.26-1.14 (m, 6H, 2×CH—CH₃). ¹³C NMR (101 MHz, Acetone-d6) δ 173.6 (C═O), 154.5 (Cquat), 152.3 (Cquat), 152.3 (Cquat), 151.1 (Cquat), 146.9 (C8), 132.1 (Cquat), 130.3 (2×Carom), 125.2 (Carom), 121.2 (2×Carom), 70.0 (C5′), 69.0 (0-CH), 57.2 (C1′), 51.2 (NH—CH), 39.0 (C4′), 35.9 (C2′), 31.8 (C3′), 27.0 (C6′), 21.9 (2×C—CH₃), 21.8 (C—CH₃). ³¹P NMR (162 MHz, Acetone-d6) δ 2.66. HRMS-ESI (m/z) [M+H]+ calcd for C₂₃H₂₈Cl₂N₅O₅P: 555.1205, found: 555.1281.

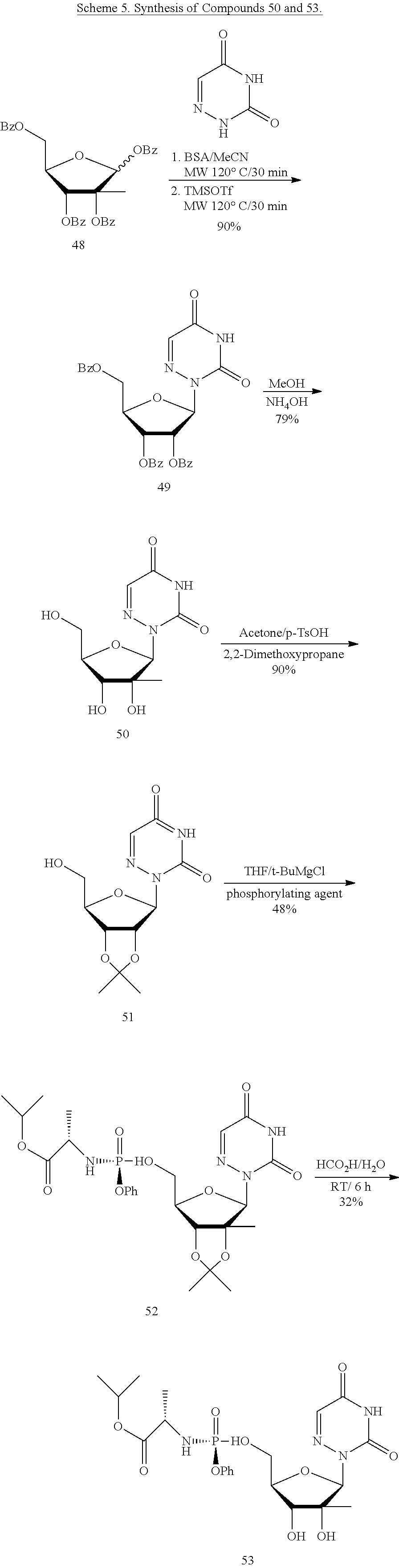

### (2R,3R,4R,5R)-5-((benzoyloxy)methyl)-2-(3,5-dioxo-4,5-dihydro-1,2,4-triazin-2(3H)-yl)-3-methyltetrahydrofuran-3,4-diyl dibenzoate (49)

6-Azauracil (565 mg, 5 mmol) was suspended in acetonitrile (5 mL) in a microwave vial, and BSA (4.5 mL) was added. The suspension was heated under microwave irradiation at 120° C. for 30 min. The solution was cooled down to rt, and compound 48 (580 mg, 1 mmol) was added, followed by TMSOTf (1 mL). The vial was heated under microwave irradiation at 120° C. for 30 min. The reaction mixture was slowly added to a saturated aq NaHCO₃ solution (50 mL) and stirred for 15 min. The mixture was then diluted with ethyl acetate (60 mL), filtered through a pad of celite and the organic layer separated. The aqueous layer was extracted with ethyl acetate (2×40 mL). The combined organic layers were washed with brine (50 mL), and concentrated under reduced pressure. The residue was purified by flash column chromatography on silica gel eluting with hexane-EtOAc (4:1 to 1:1) to give 514 mg of product (90%) as a yellow foam. ¹H-NMR (CDCl₃): □ 8.68 (s, 1H), 8.14-8.11 (m, 4H), 8.02-8.00 (m, 2H), 7.64-7.34 (m, 10H), 7.11 (s, 1H), 6.03-6.01 (d, 1H), 4.78-4.73 (m, 2H), 4.55-4.51 (m, 1H), 1.77 (s, 3H).

### 2-((2R,3R,4R,5R)-3,4-dihydroxy-5-(hydroxymethyl)-3-methyltetrahydrofuran-2-yl)-1,2,4-triazine-3,5(2H,4H)-dione (50)

To a mixture of 49 (240 mg, 0.42 mmol) in MeOH (4 mL) was added conc. NH₄OH (5 mL). The mixture was stirred at RT overnight. The volatiles were evaporated, and the residue was purified by flash chromatography on silica gel eluting with CH₂Cl₂-MeOH (9:1 to 4:1) to give 86 mg (79%) of product as a white foam. ¹H-NMR (DMSO-d6): □ 7.54 (s, 1H), 5.96 (s, 1H), 5.04 (s, br, 2H), 4.57 (s, br, 1H), 3.84-3.48 (m, 4H), 1.04 (s, 3H).

### 2-((3aR,4R,6R,6aR)-6-(hydroxymethyl)-2,2,3a-trimethyltetrahydrofuro[3,4-d][1,3]dioxol-4-yl)-1,2,4-triazine-3,5(2H,4H)-dione (51)

To a mixture of 50 (140 mg, 0.54 mmol) in dry acetone (5 mL) were added 2,2-dimethoxypropane (0.66 mL, 5.4 mmol) and p-TsOH—H₂O (124 mg, 0.65 mmol). The mixture was stirred at RT for 24 hrs. The reaction was quenched by Et₃N (2 mL), and the solvent was evaporated. The residue was purified by flash chromatography on silica gel eluting with CH₂Cl₂-MeOH (9:1) to give 145 mg (90%) of product as a white powder. ¹H-NMR (CD₃OD): Q 7.49 (s, 1H), 6.36 (s, 1H), 4.42-4.41 (d, 1H), 4.25-4.21 (m, 1H), 3.76-3.66 (m, 2H), 1.53, 1.45, 1.35 (3s, 9H).

### Isopropyl ((S)-(((3aR,4R,6R,6aR)-6-(3,5-dioxo-4,5-dihydro-1,2,4-triazin-2(3H)-yl)-2,2,6a-trimethyltetrahydrofuro[3,4-d][1,3]dioxol-4-yl)methoxy)(phenoxy)phosphoryl)-L-alaninate (52)

To a stirred mixture of 51 (132 mg, 0.44 mmol) in THE (2 mL) at 0° C. was added t-BuMgCl (1.0 M solution in THF, 0.92 mL, 0.92 mmol) dropwise, and the mixture was stirred at 0° C. for 30 min. The mixture was allowed to warmed up to RT, and was stirred at RT for another 30 min. The mixture was then cooled down to 0° C., and a solution of {(S)-2-[-(S)-(2,3,4,5,6-pentafluoro-phenoxy)-phenoxy-phosphorylamino] propionic acid isopropyl ester} (240 mg, 0.53 mmol) in THE (1 mL) was added dropwise. After addition, the reaction mixture was allowed to warmed up to RT, and stirred at RT overnight. The reaction was quenched by MeOH (0.5 mL). The solvent was evaporated, and the residue was purified by flash chromatography on silica gel eluting with CH₂Cl₂-MeOH (95:5 to 9:1) to give 120 mg (48%) of product. ¹H-NMR (CD₃OD): □ 7.49 (s, 1H), 7.37-7.33 (m, 2H), 7.24-7.16 (m, 3H), 6.35 (d, 1H), 4.98-4.93 (m, 1H), 4.38-4.20 (m, 3H), 3.89-3.76 (m, 2H), 1.51 (s, 3H), 1.45 (s, 3H), 1.34-1.31 (m, 6H), 1.22 (t, 6H).

### Isopropyl ((S)-(((2R,3R,4R,5R)-5-(3,5-dioxo-4,5-dihydro-1,2,4-triazin-2(3H)-yl)-3,4-dihydroxy-4-methyltetrahydrofuran-2-yl)methoxy)(phenoxy)phosphoryl)-L-alaninate (53)

A suspension of 52 (100 mg, 0.18 mmol) in formic acid (2 mL) and water (0.5 mL) was stirred at RT for 6 hrs. The solvent was evaporated and the residue was purified by flash chromatography on silica gel eluting with CH₂Cl₂-MeOH (9:1) to give 30 mg (32%) of product as a white foam, and 22 mg (22%) of starting material. ¹H-NMR (CD₃OD): □ 7.45 (s, 1H), 7.38-7.34 (m, 2H), 7.25-7.17 (m, 3H), 6.18 (s, 1H), 4.98-4.93 (m, 1H), 4.33-4.29 (m, 2H), 4.18-4.14 (m, 1H), 4.03-4.01 (d, 1H), 3.93-3.87 (m, 1H), 1.33 (d, 3H), 1.23 (t, 6H), 1.19 (s, 3H). HRMS calc for C₂₁H₃₀N₄O₁₀P (M+H+): 529.1700, found 529.1685.

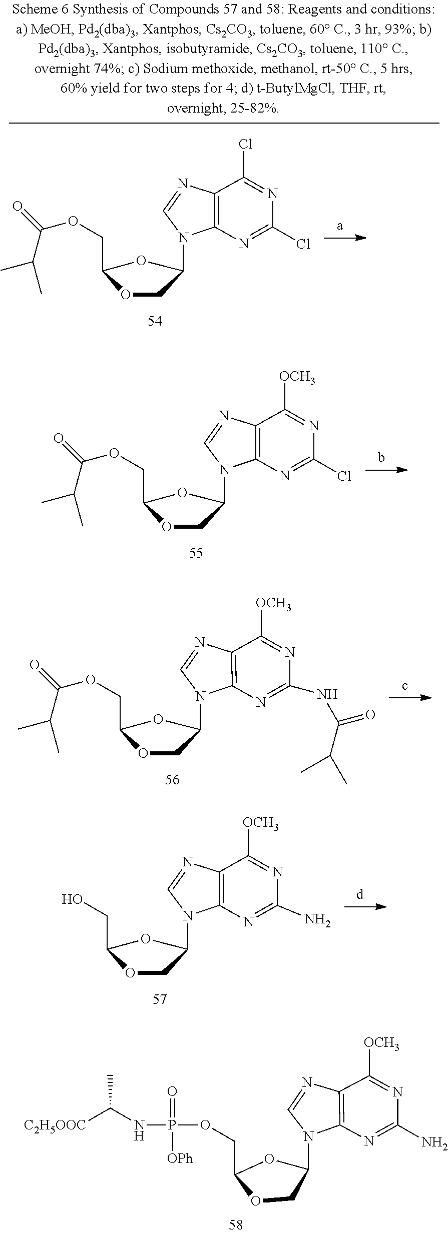

Compound 54 was prepared according to the chemistry described in:
(1) Sznaidman, M.; Painter, G. R.; Almond, M. R; Cleary, D, G.; Pesyan, A, Methods to manufacture 1,3-dioxolane nucleosides and their chiral enzymic resolution. PCT Int. Appl. 2005, WO2005074654, 98 pp.
(2)) Sznaidman, M. L.; Du, J.; Pesyan, A.; Cleary, D. G.; Hurley, P. K.; Waligora, F.; Almond, M. R. Synthesis of (−)-DAPD. Nucleosides, Nucleotides Nucleic Acids 2004, 23, 1875-1887

### ((2R,4R)-4-(2,6-dichloro-9H-purin-9-yl)-1,3-dioxolan-2-yl)methyl isobutyrate (54)

¹H NMR (CDCl₃, 400 MHz): 8.53 (s, 1H), 6.56 (d, J=4.4 Hz, 1H), 5.35 (t, J=2.8 Hz, 1H), 4.59-4.33 (m, 4H), 2.64-2.57 (m, 1H), 1.19 (d, J=6.8 Hz, 3H), 1.14 (d, J=6.8 Hz, 3H). LRMS (ESI): m/z calcd for C₁₃H₁₄Cl₂N₄O₄ (M+Na)⁺: 383.03, observed 382.9.

### ((2R,4R)-4-(2-chloro-6-methoxy-9H-purin-9-yl)-1,3-dioxolan-2-yl)methyl isobutyrate (55)

To a suspension of 54 (0.9 g, 2.49 mmol), Xantphos (112 mg, 0.19 mmol), tris(dibenzylideneacetone)dipalladium (0) (65 mg, 0.07 mmol), cesium carbonate (1.15 g, 3.52 mmol) in toluene (12 mL) under nitrogen was added methanol (0.11 mL, 2.7 mmol). The reaction mixture was heated at 60° C. for 3 hr, filtered, washed with ethyl acetate, concentrated, and purified by flash chromatography using ethyl acetate:hexane=2:1 to obtain 55 (820 mg, 93% yield). ¹H NMR (CDCl₃, 400 MHz): 8.26 (s, 1H), 6.48-6.49 (q, J=0.8 Hz, J=4.8 Hz, 1H), 5.27-5.28 (t, J=2.8 Hz, 1H), 4.46-4.49 (dd, J=0.8 Hz, J=10.0 Hz, 1H), 4.37-4.41 (dd, J=2.8 Hz, J=12.8 Hz, 1H), 4.29-4.33 (dd, J=3.2 Hz, J=12.8 Hz, 1H), 4.24-4.28 (dd, J=5.2 Hz, J=10.0 Hz, 1H), 4.17 (s, 3H), 2.54-2.61 (m, 1H), 1.13-1.15 (d, J=6.8 Hz, 3H), 1.09-1.10 (d, J=6.8 Hz, 3H). LRMS (ESI): m/z calcd for C₁₄H₁₈ClN₄O₅ (M+H)⁺: 357.09 observed 356.88.

### ((2R,4R)-4-(2-isobutyramido-6-methoxy-9H-purin-9-yl)-1,3-dioxolan-2-yl)methyl isobutyrate (56)

A suspension of 55 (112 mg, 0.31 mmol), tris(dibenzylideneacetone)dipalladium(0) (6.5 mg, 0.007 mmol), xantphos (11.2 mg, 0.019 mmol), cesium carbonate (115 mg, 0.35 mmol) and isobutyramide (33.5 mg, 0.38 mmol) in toluene (2 mL) was heated at 110° C. for 12 h, then filtered through celite, concentrated under vacuum and purified by flash chromatography to give 56 (94 mg, 74% yield). ¹H NMR (CDCl₃, 400 MHz): 8.11 (s, 1H), 8.10 (brs, 1H), 6.42-6.43 (d, J=4.0 Hz, 1H), 5.24-5.25 (t, J=2.8 Hz, 1H), 4.47-4.50 (dd, J=1.2 Hz, J=10.0 Hz, 1H), 4.312-4.319 (d, J=2.8 Hz, 2H), 4.21-4.25 (dd, J=5.6 Hz, J=10.0 Hz, 1H), 4.07 (s, 3H), 3.10 (brs, 1H), 2.50-2.57 (m, 1H), 1.22-1.24 (2d, J=0.8 Hz, 6H), 1.05-1.12 (2d, J=7.2 Hz, 6H). LRMS (ESI): m/z calcd for C₁₈H₂₆N₅O₆ (M+H)⁺: 408.18 observed 407.98.

### ((2R,4R)-4-(2-amino-6-methoxy-9H-purin-9-yl)-1,3-dioxolan-2-yl)methanol (57)

To a solution of 56 (4.98 mmol) in with methanol (10 mL) was added sodium methoxide (25%, 3 mL). The solution was stirred for 5 h at room temperature and then heated to 50° C. for 30 min. After evaporation under vacuum, the residue was purified by column chromatography (Dichloromethane:methanol=60:3) to afford 57 (800 mg, 60%). ¹H NMR (CDCl₃, 400 MHz): 8.12 (s, 1H), 6.30 (dd, J=1.6 Hz, J=5.6 Hz, 1H), 5.08 (t, J=2.8 Hz, 1H), 4.46 (dd, J=1.6 Hz, J=10.0 Hz, 1H), 4.21 (dd, J=5.2 Hz, J=9.6 Hz, 1H), 3.75 (t, J=2.0 Hz, 2H). LRMS (ESI): m/z calcd for C₁₀H14N₅O₄ (M+H)⁺: 268.10, observed 268.04.

### Ethyl ((((2R,4R)-4-(2-amino-6-methoxy-9H-purin-9-yl)-1,3-dioxolan-2-yl)methoxy)(phenoxy)phosphoryl)-L-alaninate (58)

A solution of 57 (50 mg, 0.18 mmol) in THE (3 mL) was added t-butylmagnesium chloride (0.54 mL, 1 M in THF, 0.54 mmol). The reaction mixture was stirred at rt for 30 min, before addition of the corresponding phosphoramide chloride (0.54 mL, 1 M in THF, 0.54 mmol). The reaction mixture was stirred overnight, quenched with saturated ammonium chloride (1 mL) and directly purified by column chromatography (dichloromethane:methanol=100:1 to 100:5) to afford the desired compound.

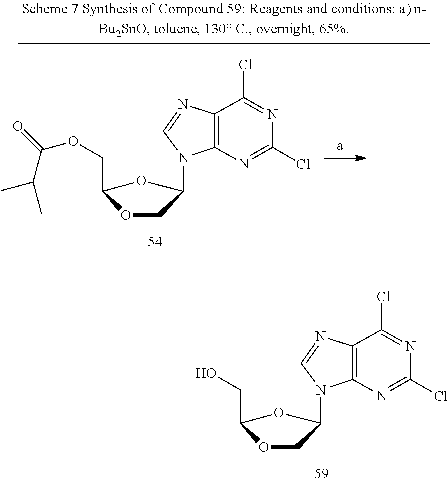

### ((2R,4R)-4-(2,6-dichloro-9H-purin-9-yl)-1,3-dioxolan-2-yl)methanol (59)

A suspension of dichloropurine 54 (3 g, 8.30 mmol) and dibutyltin (IV) oxide (6 g, 24.1 mmol) in toluene (50 mL) was heated to 130° C. overnight. After evaporation of the volatiles under vacuum, the residue was directly purified by column chromatography (Dichloromethane:methanol=100:1 to 100:10) to afford 59 (65%). ¹H NMR (CD₃OD, 400 MHz): 8.90 (s, 1H), 6.57 (d, J=5.0 Hz, 1H), 5.17 (t, J=2.0 Hz, 1H), 4.65 (d, J=10.1 Hz, 1H), 4.35-4.31 (dd, J=5.0 Hz, J=10.1 Hz, 1H), 3.86-3.77 (m, 2H). LRMS (ESI): m/z calcd for C₉H₈Cl₂N₄O₃ (M+Na)⁺: 312.99, observed 312.9.

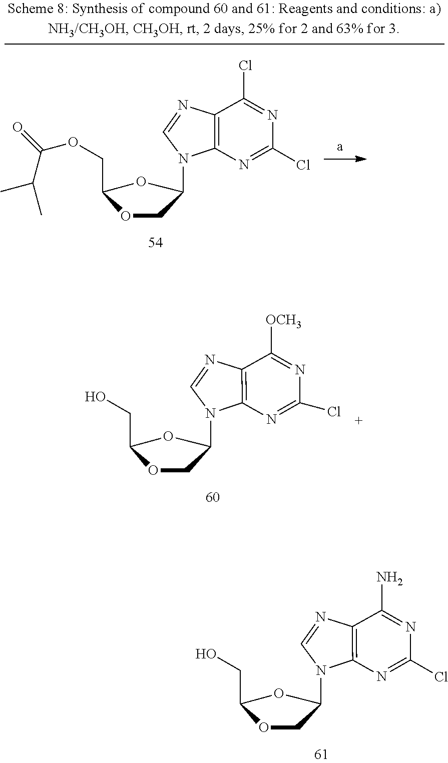

### ((2R,4R)-4-(2-chloro-6-methoxy-9H-purin-9-yl)-1,3-dioxolan-2-yl)methanol (60) and ((2R,4R)-4-(6-amino-2-chloro-9H-purin-9-yl)-1,3-dioxolan-2-yl)methanol (61)

A solution of 54 (1 g, 2.76 mmol) in NH₃/CH₃OH (10 mL) was stirred for 2 days at room temperature. After evaporation of the volatiles, the residue was purified flash chromatography (Dichloromethane:methanol=100:1 to 100:10) to afford 60 (35%) and 61 (63%).

(60): ¹H NMR (CD₃OD, 400 MHz): 8.61 (s, 1H), 6.51-6.49 (dd, J=0.8 Hz, J=4.8 Hz, 1H), 5.15 (t, J=2.4 Hz, 1H), 4.59-4.56 (dd, J=0.8 Hz, J=10.0 Hz, 1H), 4.33-4.29 (dd, J=5.2 Hz, J=10.0 Hz, 1H), 4.16 (s, 3H), 3.84-3.80 (m, 2H). LRMS (ESI): m/z calcd for C₁₀H₁₁ClN₄O₄ (M+Na)⁺: 309.04, observed 309.0.

(61): ¹H NMR (CD₃OD, 400 MHz): 8.42 (s, 1H), 6.41-6.40 (dd, J=4.4 Hz, 1H), 5.13 (d, J=2.4 Hz, 1H), 4.52-4.49 (d, J=10.0 Hz, 1H), 4.30-4.26 (dd, J=5.2 Hz, J=10.0 Hz, 1H), 3.83-3.75 (m, 2H). LRMS (ESI): m/z calcd for C₉H₁₀ClN₅O₃ (M+Na)⁺: 294.03, observed 294.0.

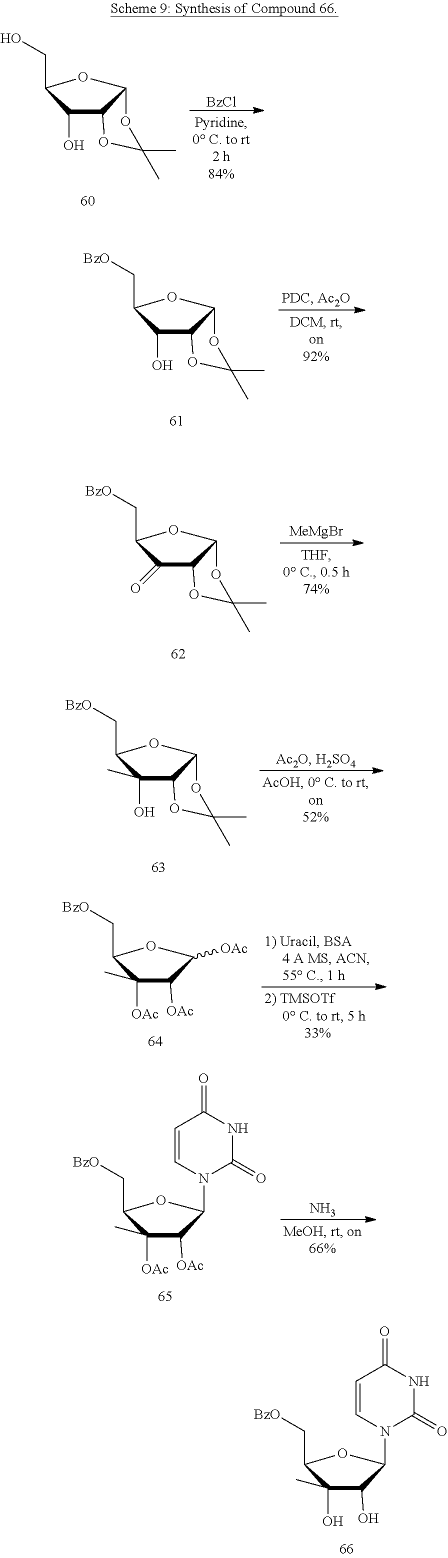

3′Me sugar 64 was synthetized following. WO2019090111 A1 and Tetrahedron, 2002, 58, 9593. 3′Me-Uridine 66 was synthetized following Bioorg. Med. Chem. 2008, 16, 6319 and Biochemistry 1992, 31, 45, 11210-11215.

1-((2R,3R,4R,5R)-3,4-dihydroxy-5-(hydroxymethyl)-4-methyltetrahydrofuran-2-yl)pyrimidine-2,4(1H,3H)-dione 66 as a white foam (66%). ¹H NMR (400 MHz, Methanol-d4) δ 8.19 (d, 1H, J=8.1 Hz), 6.05 (d, 1H, J=7.8 Hz), 5.73 (d, 1H, J=8.0 Hz), 4.05 (d, 1H, J=7.8 Hz), 3.95 (t, 1H, J=2.2 Hz), 3.80-3.68 (m, 2H), 1.38 (s, 3H). ¹³C NMR (101 MHz, Methanol-d4) δ 164.7, 151.5, 141.9, 101.5, 87.8 87.2, 77.5 76.6, 60.8, 18.7. HRMS-ESI (m/z) [M+H]⁺ calcd. 259.0852. for C₁₀H₁₅N₂O₆: found 259.0930.

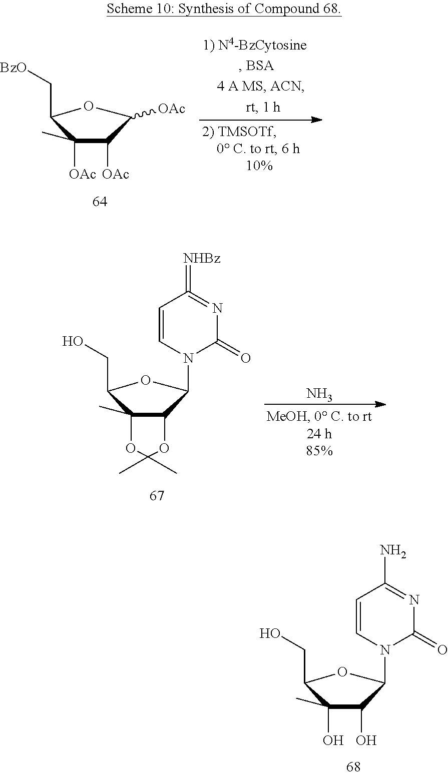

(2R,3S,4R,5R)-5-(4-benzamido-2-oxopyrimidin-1(2H)-yl)-2-((benzoyloxy)methyl)-3-methyltetrahydrofuran-3,4-diyl diacetate 67: 4-N-benzoyl-cytosine (1.49 g, 4.8 mmol, 1.2 eq.) and N,O-bistrimethylsilylacetamide (1.9 mL, 8.00 mmol, 2 eq.) were heated at 55° C. in acetonitrile (12 mL) until a clear solution was observed. Then (3R,4S,5R)-5-((benzoyloxy)methyl)-4-methyltetrahydrofuran-2,3,4-triyl triacetate 64 (1.58 g, 4 mmol, 1 eq.) was added and the mixture was cooled to 0° C. Trimethylsilyl triflate (2.2 mL, 12 mmol, 3 eq.) was added dropwise, and the mixture was stirred at room temperature for 6 h. The reaction mixture was then poured into sat NH₄Cl (100 mL), extracted with dichloromethane (3×50 mL). The organic layers were combined, washed with brine (50 ml), dried over MgSO₄, filtered, and concentrated in vacuo to dryness. The crude product was purified by flash chromatography (Hexane/Ethyl acetate 100/0 to 0/100) to give the title compound (210 mg, 10%) as a white foam. ¹H NMR (400 MHz, Acetone-d6) δ 8.20-8.11 (m, 3H), 8.11-8.06 (m, 2H), 7.72-7.60 (m, 2H), 7.55 (q, 4H, J=7.5 Hz), 7.29 (s, 1H), 6.26 (d, 1H, J=6.9 Hz), 5.57 (d, 1H, J=7.0 Hz), 4.94 (dd, 1H, J=5.3, 3.9 Hz), 4.82-4.64 (m, 2H), 2.14 (s, 3H), 2.09 (s, 3H), 1.77 (s, 3H). ¹³C NMR (101 MHz, Acetone-d6) δ 170.5, 170.4, 166.4, 163.7, 145.2, 134.3, 133.7, 130.6, 130.33, 129.6, 12.4, 129.1, 87.7, 84.5, 82.2, 78.9, 64.3, 21.8, 20.5, 17.9.

4-amino-1-((2R,3R,4R,5R)-3,4-dihydroxy-5-(hydroxymethyl)-4-methyltetrahydrofuran-2-yl)pyrimidin-2(1H)-one 68. To a solution of compound 67 (0.21 g, mmol, eq.) in methanol (8 ml) was bubbled NH_{3(g)}. The reaction was stirred at room temperature for 24 hours, then evaporated and purified by flash chromatography (Dichloromethane/Methanol 100/0 to 85/15) to give the title compound (88.8 mg, 85%) as a white foam. ¹H NMR (400 MHz, Methanol-d4) δ 8.03 (d, 1H, J=7.5 Hz), 5.95 (d, 1H, J=7.5 Hz), 5.89 (d, 1H, J=7.5 Hz), 4.07 (d, 1H, J=7.5 Hz), 3.93 (t, 1H, J=2.9 Hz), 3.77-3.60 (m, 2H), 1.34 (s, 3H). ¹³C NMR (101 MHz, Methanol-d4) δ 166.1, 157.6, 142.7, 94.9, 89.4, 87.8, 78.0, 76.7, 60.9, 47.3, 47.1, 46.9, 18.8. HRMS-ESI (m/z) [M+H]⁺ calcd. 258.1012. for C₁₀H₁₆N₃O₅: found 258.1089.

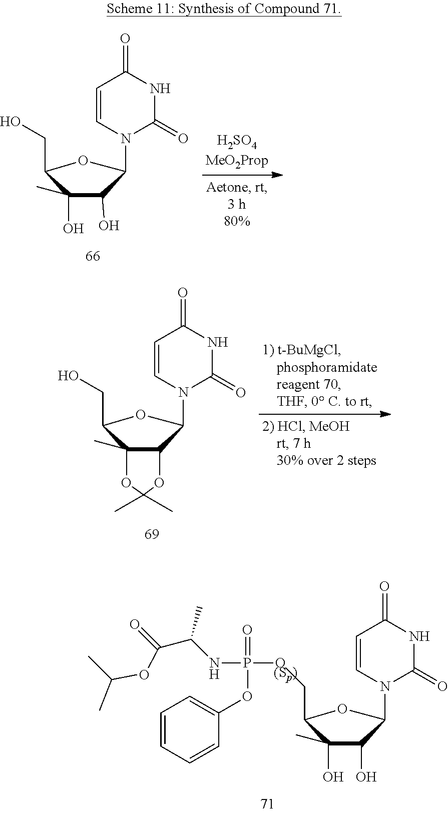

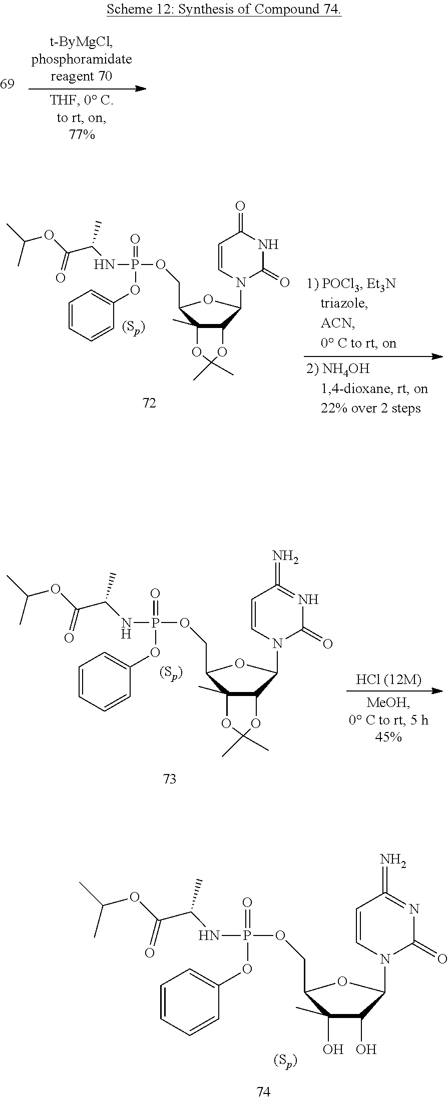

(1-((3aR,4R,6R,6aS)-6-(hydroxymethyl)-2,2,6a-trimethyltetrahydrofuro[3,4-d][1,3]dioxol-4-yl)pyrimidine-2,4(1H,3H)-dione 69. To a solution of 66 (55 mg, 0.21 mmol, 1 eq.) in acetone (4.2 ml) was added dimethoxy propane (0.11 ml, 0.84 mmol, 4 eq.) and conc. sulfuric acid (0.002 ml, 0.042 mmol, 0.2 eq.) at room temperature. The resulting reaction mixture was stirred 3 hours. Then, sodium carbonate was added and the resulting mixture was filtered and volatiles were removed under vacuum. The residue was purified by flash chromatography (Dichloromethane/Methanol, 100/0 to 90/10) to give the title compound (50.1 mg, 80%) as a white foam. ¹H NMR (400 MHz, Methanol-d4) δ 7.79 (d, 1H, J=8.0 Hz), 5.86 (d, 1H, J=2.1 Hz), 5.73 (d, 1H, J=8.0 Hz), 4.53 (d, 1H, J=2.1 Hz), 4.12-3.10 (m, 1H), 3.82-3.70 (m, 2H), 1.55 (s, 3H), 1.48 (s, 3H), 1.41 (s, 3H). ¹³C NMR (101 MHz, Methanol-d4) δ 164.7, 150.5, 141.3, 114.0, 99.6, 90.2, 89.9, 87.7, 87.3, 58.3, 27.1, 26.2, 17.3. HRMS-ESI (m/z) [M+H]⁺ calcd. 299.1195. for C₁₃H₁₉N₂O₆: found 299.1244.

isopropyl ((((2R,3R,4R,5R)-5-(2,4-dioxo-3,4-dihydropyrimidin-1(2H)-yl)-3,4-dihydroxy-3-methyltetrahydrofuran-2-yl)methoxy)(phenoxy)phosphoryl)-L-alaninate 71. Under inert atmosphere, compound 31 (0.21 g, 0.7 mmol, 1 eq.) was dissolved into anhydrous THE (2.7 ml, 0.26M), with 4 Å molecular thieves. The mixture was cooled down to 0° C. and t-BuMgCl (1M in THF, 2.18 ml, 2.18 mmol, 3.1 eq.) was added dropwise. The reaction was left 30 min at 0° C. and then 30 min at room temperature. The reaction was cooled down to 0° C. and a solution of {(S)-2-[-(S)-(2,3,4,5,6-pentafluoro-phenoxy)-phenoxy-phosphorylamino]propionic acid isopropyl ester} 70 (0.48 g, 1.05 mmol, 1.5 eq.) in dry THE (2.1 ml, 0.5M) was added. The reaction was stirred overnight at room temperature and after completion of the reaction, HCl (12 M, 20 eq.) was added dropwise at 0° C. The mixture was stirred at room temperature for 7 h. After addition of a solution of ammonia in methanol (10 ml), volatiles were removed under vacuum. The resulting residue was purified by flash chromatography (Dichloromethane/Methanol, 100/0 to 90/10) to give the title compound (111 mg, 30% over 2 steps) as a white foam. ¹H NMR (400 MHz, DMSO-d6) 11.37 (s, 1H), 7.69 (d, 1H, J=8.1 Hz), 7.38 (t, 2H, J=7.8 Hz), 7.26-7.15 (m, 3H), 6.09 (dd, 1H, J=12.9, 10.0 Hz), 5.86 (d, 1H, J=8.0 Hz), 5.56 (d, 1H, J=8.1 Hz), 5.50 (d, 1H, J=6.3 Hz), 5.00 (s, 1H), 4.86 (p, 1H, J=6.3 Hz), 4.14 (dt, 1H, J=10.2, 4.8 Hz), 4.05 (dt, 1H, J=10.9, 4.7 Hz), 3.96 (d, 1H, J=4.4 Hz), 3.86-3.74 (m, 2H), 1.22 (d, J=7.0 Hz, 3H), 1.19-1.12 (m, 9H). ¹³C NMR (101 MHz, DMSO-d₆) δ 172.5, 162.9, 151.0, 150.6, 140.7, 129.6, 124.6, 120.1, 102.1, 85.6, 84.5, 75.7, 75.6, 68.0, 65.5, 49.7, 21.4, 21.3, 19.8. ³¹P NMR (162 MHz, DMSO-d6) δ 3.68.

Isopropyl ((((3aS,4R,6R,6aR)-6-(2,4-dioxo-3,4-dihydropyrimidin-1(2H)-yl)-2,2,3a-trimethyltetrahydrofuro[3,4-d][1,3]dioxol-4-yl)methoxy)(phenoxy)phosphoryl)-L-alaninate 72. Under inert atmosphere, isopropylidene derivative 69 (0.21 g, 0.7 mmol, 1 eq.) was dissolved into anhydrous THE (2.7 ml, 0.26M), with 4 Å molecular thieves. The mixture was cooled down to 0° C. and t-BuMgCl (1M in THF, 2.18 ml, 2.18 mmol, 3.1 eq.) was added dropwise. The reaction was left 30 min at 0° C. and then 30 min at room temperature. A solution of {(S)-2-[-(S)-(2,3,4,5,6-pentafluoro-phenoxy)-phenoxy-phosphorylamino] propionic acid isopropyl ester} 70 (0.48 g, 1.05 mmol, 1.5 eq.) in dry THF (2.1 ml, 0.5M). was added dropwise to the reaction. After being stirred overnight at room temperature, the reaction was diluted with a saturated solution of NaHCO₃ (25 ml) and extracted with ethyl acetate (3×20 ml). The organic layers were combined, washed with brine (20 ml), dried over MgSO₄, filtered, and concentrated in vacuo to dryness. The residue was purified by flash chromatography (Dichloromethane/Methanol, 100/0 to 90/10) to give the title compound (300 mg, 77%) as a white foam. ¹H NMR (400 MHz, Acetone-d₆) δ 10.15 (s, 1H), 7.68 (d, 1H, J=8.1 Hz), 7.41-7.33 (m, 2H), 7.29 (dt, 2H, J=7.6, 1.3 Hz), 7.18 (ddq, 1H, J=8.6, 7.6, 1.1 Hz), 5.87 (d, 1H, J=2.2 Hz), 5.63 (dq, 1H, J=8.1, 1.0 Hz), 4.95 (hept, 1H, J=6.3 Hz,), 4.85 (s, 1H), 4.65 (d, 1H, J=2.2 Hz), 4.38-4.23 (m, 1H), 4.27-4.18 (m, 2H), 3.94 (tq, 1H, J=9.8, 7.1 Hz), 1.52 (d, 6H, J=3.8 Hz), 1.38 (s, 3H), 1.34 (dd, 3H, J=7.1, 0.9 Hz), 1.20 (dd, 6H, J=6.2, 1.8 Hz). ¹³C NMR (101 MHz, Acetone-d₆) 183.2, 173.1, 161.8, 160.7, 151.5, 140.0, 135.1, 130.9, 124.7, 112.6, 100.9, 100.0, 97.8, 96.0, 78.8, 74.97, 60.8, 37.9, 37.0, 31.5, 30.4, 29.3. ³¹P NMR (162 MHz, Acetone-d6) δ 2.95. HRMS-ESI (m/z) [M+H]⁺ calcd. 568.1982. for C₂₅H₃₅N₃O₁₀P: found 568.2072.

isopropyl ((((3aS,4R,6R,6aR)-6-(4-amino-2-oxopyrimidin-1(2H)-yl)-2,2,3a-trimethyltetrahydrofuro[3,4-d][1,3]dioxol-4-yl)methoxy)(phenoxy)phosphoryl)-L-alaninate 73 To a solution of 1,2,4-triazole (750 mg, 10.85 mmol, 31 eq.) in acetonitrile (20 ml, 0.088M) was added. Et₃N (1.65 mL, 11.69 mmol, 33.4 eq.) and phosphoryl chloride (0.17 mL, 1.82 mmol, 5.2 eq.) at 0° C. The mixture was stirred at 0° C. for 3 hours before addition of a solution of 72 (200 mg, 0.8 mmol, 1 eq.) in ACN (4.5 ml, 0.35M). The reaction mixture was stirred overnight at room temperature. The reaction was then diluted with ethyl acetate (100 ml), filtered off, washed with sat NaHCO₃ (20 ml) and brine (20 ml). The organic layer was concentrated under vacuum and the residue purified by flash column chromatography (Hexane/Ethyl acetate, 100/0 to 20/80) to afford the desired triazole intermediate. To a solution of this intermediate in 1,4-dioxane (3 mL) was added aqueous ammonia (0.5 mL) at room temperature. The resulting reaction mixture was stirred for 2.5 hours before evaporation of the volatiles under vacuum. The residue was purified by flash chromatography (Dichloromethane/Methanol, 100/0 to 90/10) to give the title compound (42.1 mg, 22% over 2 steps) as a white foam. ¹H NMR (400 MHz, Methanol-d₄) δ 7.70 (d, 1H, J=7.5 Hz), 7.35 (t, 2H, J=7.8 Hz), 7.28-7.22 (m, 2H), 7.19 (t, 1H, J=7.4 Hz), 5.89 (d, 1H, J=7.5 Hz), 5.82 (d, 1H, J=1.8 Hz), 4.96 (h, 1H, J=6.2 Hz), 4.47 (d, 1H, J=1.9H), 4.28 (dddd, 3H, J=24.9, 12.1, 6.6, 3.5 Hz), 3.89 (dq, 1H, J=9.6, 7.1 Hz), 1.52 (s, 3H), 1.43 (s, 3H), 1.37 (s, 3H), 1.33 (d, 3H, J=7.1 Hz), 1.21 (d, 6H, J=6.3 Hz). ¹³C NMR (101 MHz, Methanol-d₄) δ 173.0, 166.4, 156.4, 150.7, 141.6, 129.3, 124.8, 120.1, 120.0, 114.1, 94.8, 91.2, 90.0, 86.9, 85.5, 85.4, 68.7, 64.8, 50.2, 26.9, 25.8, 20.5, 19.0, 18.2. ³¹P NMR (162 MHz, Methanol-d₄) δ 3.71.

isopropyl ((((2R,3R,4R,5R)-5-(4-amino-2-oxopyrimidin-1(2H)-yl)-3,4-dihydroxy-3-methyltetrahydrofuran-2-yl)methoxy)(phenoxy)phosphoryl)-L-alaninate 74. Compound 73 (43 mg, 0.076 mmol, 1 eq.) was dissolved in methanol (2 ml) and HCl (12 M, 0.3 ml) was added dropwise at 0° C. The mixture was stirred at room temperature for 5 h. before addition of a saturated solution of ammonia in methanol (10 ml). Volatiles were removed under vacuum and the residue purified by flash chromatography (Dichloromethane/Methanol, 100/0 to 85/15) to give the title compound (17.4 mg, 45%) as a white foam. ¹H NMR (400 MHz, Methanol-d₄) δ 7.68 (d, 1H, J=7.6 Hz), 7.27 (dd, 2H, J=8.8, 7.2 Hz), 7.19-7.07 (m, 3H), 5.92 (d, 1H, J=7.3 Hz), 5.82-5.70 (m, 1H), 4.86 (p, 1H, J=6.3 Hz,), 4.28-4.08 (m, 2H), 4.04-4.00 (m, 1H), 3.82-373 (m, 2H), 1.26-1.17 (m, 6H), 1.12 (dd, J=6.2, 2.2 Hz, 6H).). ¹³C NMR (101 MHz, Methanol-d₄) δ 172.8, 166.0, 157.7, 150.6, 150.5, 141.3, 129.6, 129.5, 124.9, 120.0, 95.4, 88.3, 85.1, 78.2, 76.3, 68.8, 65.8, 50.3, 20.5, 20.4, 19.2, 18.9. ³¹P NMR (162 MHz, Methanol-d₄) δ 3.59.

### Example 2

### Cellular Toxicity Assays

The toxicity of the compounds was assessed in Vero, human PBM, CEM (human lymphoblastoid), MT-2, and HepG2 cells, as described previously (see Schinazi R. F., Sommadossi J.-P., Saalmann V., Cannon D. L., Xie M.-Y., Hart G. C., Smith G. A. & Hahn E. F. Antimicrob. Agents Chemother. 1990, 34, 1061-67). Cycloheximide was included as positive cytotoxic control, and untreated cells exposed to solvent were included as negative controls. The cytotoxicity IC50 was obtained from the concentration-response curve using the median effective method described previously (see Chou T.-C. & Talalay P. Adv. Enzyme Regul. 1984, 22, 27-55; Belen'kii M. S. & Schinazi R. F. Antiviral Res. 1994, 25, 1-11).

### Example 3

### Mitochondrial Toxicity Assays in HepG2 Cells:

i) Effect of Compounds on Cell Growth and Lactic Acid Production: The effect on the growth of HepG2 cells can be determined by incubating cells in the presence of 0 μM, 0.1 μM, 1 μM, 10 μM and 100 μM drug. Cells (5×10⁴ per well) can be plated into 12-well cell culture clusters in minimum essential medium with nonessential amino acids supplemented with 10% fetal bovine serum, 1% sodium pyruvate, and 1% penicillin/streptomycin and incubated for 4 days at 37° C. At the end of the incubation period the cell number can be determined using a hemocytometer. Also taught by Pan-Zhou X-R, Cui L, Zhou X-J, Sommadossi J-P, Darley-Usmer V M. “Differential effects of antiretroviral nucleoside analogs on mitochondrial function in HepG2 cells,” Antimicrob. Agents Chemother. 2000; 44: 496-503.

To measure the effects of the compounds on lactic acid production, HepG2 cells from a stock culture can be diluted and plated in 12-well culture plates at 2.5×10⁴ cells per well. Various concentrations (0 μM, 0.1 μM, 1 μM, 10 μM and 100 μM) of compound can be added, and the cultures can be incubated at 37° C. in a humidified 5% CO₂ atmosphere for 4 days. At day 4, the number of cells in each well can be determined and the culture medium collected. The culture medium can then be filtered, and the lactic acid content in the medium determined using a colorimetric lactic acid assay (Sigma-Aldrich). Since lactic acid product can be considered a marker for impaired mitochondrial function, elevated levels of lactic acid production detected in cells grown in the presence of test compounds indicates a drug-induced cytotoxic effect.

ii) Effect on Compounds on Mitochondrial DNA Synthesis: a real-time PCR assay to accurately quantify mitochondrial DNA content has been developed (see Stuyver L J, Lostia S, Adams M, Mathew J S, Pai B S, Grier J, Tharnish P M, Choi Y, Chong Y, Choo H, Chu C K, Otto M J, Schinazi R F. Antiviral activities and cellular toxicities of modified 2′,3′-dideoxy-2′,3′-didehydrocytidine analogs. Antimicrob. Agents Chemother. 2002; 46: 3854-60). This assay can be used in all studies described in this application that determine the effect of compounds on mitochondrial DNA content. In this assay, low-passage-number HepG2 cells are seeded at 5,000 cells/well in collagen-coated 96-well plates. Test compounds are added to the medium to obtain final concentrations of 0 μM, 0.1 μM, 10 μM and 100 μM. On culture day 7, cellular nucleic acids can be prepared by using commercially available columns (RNeasy 96 kit; Qiagen). These kits co-purify RNA and DNA, and hence, total nucleic acids are eluted from the columns. The mitochondrial cytochrome c oxidase subunit II (COXII) gene and the 8-actin or rRNA gene can be amplified from 5 μl of the eluted nucleic acids using a multiplex Q-PCR protocol with suitable primers and probes for both target and reference amplifications. For COXII the following sense, probe and antisense primers can be used, respectively: 5′-TGCCCGCCATCATCCTA-3′, 5′-tetrachloro-6-carboxyfluorescein-TCCTCATCGCCCTCCCATCCC-TAMRA-3′ and 5′-CGTCTGTTATGTAAAGGATGCGT-3′. For exon 3 of the β-actin gene (GenBank accession number E01094) the sense, probe, and antisense primers are 5′-GCGCGGCTACAGCTTCA-3′, 5′-6-FAMCACCACGGCCGAGCGGGATAMRA-3′ and 5′-TCTCCTTAATGTCACGCACGAT-3′, respectively. The primers and probes for the rRNA gene are commercially available from Applied Biosystems. Since equal amplification efficiencies are obtained for all genes, the comparative CT method can be used to investigate potential inhibition of mitochondrial DNA synthesis. The comparative CT method uses arithmetic formulas in which the amount of target (COXII gene) is normalized to the amount of an endogenous reference (the β-actin or rRNA gene) and is relative to a calibrator (a control with no drug at day 7). The arithmetic formula for this approach is given by 2-ΔΔCT, where ΔΔCT is (CT for average target test sample−CT for target control)−(CT for average reference test−CT for reference control) (see Johnson M R, K Wang, J B Smith, M J Heslin, R B Diasio. Quantitation of dihydropyrimidine dehydrogenase expression by real-time reverse transcription polymerase chain reaction. Anal. Biochem. 2000; 278:175-184). A decrease in mitochondrial DNA content in cells grown in the presence of drug indicates mitochondrial toxicity.

### Example 4

### Mitochondrial Toxicity-Glu/Gal

### Protocol Summary

HepG2 cells are plated on 96 or 384 well tissue culture polystyrene plates. After 24 hr the cells are dosed with test compound at a range of concentrations and incubated for 72 hr in medium supplemented with either galactose or glucose. Test compounds are said to cause mitochondrial toxicity if the cells grown in galactose-containing medium are more sensitive to the test compound than the cells grown in glucose-containing medium.

Objective: To measure the sensitivity of HepG2 cells grown in medium containing either galactose or glucose to the test compound.

### Experimental Procedure

HepG2 human hepatocellular carcinoma cells are plated on 96 or 384-well tissue culture polystyrene plates containing either galactose or glucose containing medium supplemented with 10% fetal bovine serum and antibiotics and incubated overnight. The cells are dosed with increasing concentrations of the test compound (final DMSO concentration 0.5%; typical final test compound concentrations of 100, 30, 10, 3, 1, 0.3, 0.1, 0.03 μM for an eight point dose response curve; n=3 replicates per concentration) and the cells are incubated for 72 hr. Appropriate controls are simultaneously used as quality controls. Cell viability is measured using Hoechst staining and cell counting by a HCS reader.

### Example 5

### Mitochondrial Toxicity Assays in Neuro2A Cells

To estimate the potential of the compounds described herein to cause neuronal toxicity, mouse Neuro2A cells (American Type Culture Collection 131) can be used as a model system (see Ray A S, Hernandez-Santiago B I, Mathew J S, Murakami E, Bozeman C, Xie M Y, Dutschman G E, Gullen E, Yang Z, Hurwitz S, Cheng Y C, Chu C K, McClure H, Schinazi R F, Anderson K S. Mechanism of anti-human immunodeficiency virus activity of beta-D-6-cyclopropylamino-2′,3′-didehydro-2′,3′-dideoxyguanosine. Antimicrob. Agents Chemother. 2005, 49, 1994-2001). The concentrations necessary to inhibit cell growth by 50% (CC50) can be measured using the 3-(4,5-dimethyl-thiazol-2-yl)-2,5-diphenyltetrazolium bromide dye-based assay, as described. Perturbations in cellular lactic acid and mitochondrial DNA levels at defined concentrations of drug can be carried out as described above. ddC and AZT can be used as control nucleoside analogs.

### Example 6

### Assay for Bone Marrow Cytotoxicity

Primary human bone marrow mononuclear cells can be obtained commercially from Cambrex Bioscience (Walkersville, MD). CFU-GM assays is carried out using a bilayer soft agar in the presence of 50 units/mL human recombinant granulocyte/macrophage colony-stimulating factor, while BFU-E assays used a ethylcellulose matrix containing 1 unit/mL erythropoietin (see Sommadossi J P, Carlisle R. Toxicity of 3′-azido-3′-deoxythymidine and 9-(1,3-dihydroxy-2-propoxymethyl) guanine for normal human hepatopoietic progenitor cells in vitro. Antimicrob. Agents Chemother. 1987; 31: 452-454; Sommadossi, J P, Schinazi, R F, Chu, C K, and Xie, M Y. Comparison of cytotoxicity of the (−) and (+) enantiomer of 2′,3′-dideoxy-3′-thiacytidine in normal human bone marrow progenitor cells. Biochem. Pharmacol. 1992; 44:1921-1925). Each experiment can be performed in duplicate in cells from three different donors. AZT is used as a positive control. Cells can be incubated in the presence of the compound for 14-18 days at 37° C. with 5% CO₂, and colonies of greater than 50 cells can be counted using an inverted microscope to determine the IC₅₀. The 50% inhibitory concentration (IC₅₀) can be obtained by least-squares linear regression analysis of the logarithm of drug concentration versus BFU-E survival fractions. Statistical analysis can be performed with Student's t test for independent non-paired samples.

### Example 7

### In Vitro Human Mitochondrial RNA Polymerase (POLRMT) Assay

In vitro RNA nucleotide incorporation assays with POLRMT (INDIGO Biosciences) can be performed as previously described (Arnold et al. 2012). Briefly, ³²P-radiolabeled RNA primer (5′-UUUUGCCGCGCC) can be hybridized to 3 molar excess of the appropriate DNA template (5′-GGGAATGCANGGCGCGGC where position N can be replaced by A, T, or C). 125 nM of POLRMT can be incubated with 500 nM of 5′-radiolabled RNA/DNA hybrid, 10 mM MgCl₂ and 100 μM of the corresponding nucleoside triphosphate. For non-nucleoside analogs, 100 μM of inhibitor can be added at the same time as 100 μM UTP. Incorporation can be allowed to proceed for 2 h at 30° C. and reactions are stopped by the addition of 10 mM EDTA and formamide. Samples are visualized on 20% denaturing polyacrylamide gel. Data can be analyzed by normalizing the product fraction for each nucleoside triphosphate analog to that of the corresponding natural nucleoside triphosphate.

### Example 8

### Effect of Nucleotide Analogs on the DNA Polymerase and Exonuclease Activities of Mitochondrial DNA Polymerase γ

i) Purification of Human Polymerase γ: The recombinant large and small subunits of polymerase γ can be purified as described previously (see Graves S W, Johnson A A, Johnson K A. Expression, purification, and initial kinetic characterization of the large subunit of the human mitochondrial DNA polymerase. Biochemistry. 1998, 37, 6050-8; Johnson A A, Tsai Y, Graves S W, Johnson K A. Human mitochondrial DNA polymerase holoenzyme: reconstitution and characterization. Biochemistry 2000; 39: 1702-8). The protein concentration can be determined spectrophotometrically at 280 nm, with extinction coefficients of 234,420, and 71,894 M-1 cm-1 for the large and the small subunits of polymerase γ, respectively.

ii) Kinetic Analyses of Nucleotide Incorporation: Pre-steady-state kinetic analyses can be performed to determine the catalytic efficiency of incorporation (k/K) for DNA polymerase γ for nucleoside-TP and natural dNTP substrates. This allowed determination of the relative ability of this enzyme to incorporate modified analogs and predict toxicity. Pre-steady-state kinetic analyses of incorporation of nucleotide analogs by DNA polymerase γ would be carried out essentially as described previously (see Murakami E, Ray A S, Schinazi R F, Anderson K S. Investigating the effects of stereochemistry on incorporation and removal of 5-fluorocytidine analogs by mitochondrial DNA polymerase gamma: comparison of D- and L-D4FC-TP. Antiviral Res. 2004, 62, 57-64; Feng J Y, Murakami E, Zorca S M, Johnson A A, Johnson K A, Schinazi R F, Furman P A, Anderson K S. Relationship between antiviral activity and host toxicity: comparison of the incorporation efficiencies of 2′,3′-dideoxy-5-fluoro-3′-thiacytidine-triphosphate analogs by human immunodeficiency virus type 1 reverse transcriptase and human mitochondrial DNA polymerase. Antimicrob Agents Chemother. 2004, 48, 1300-6). Briefly, a pre-incubated mixture of large (250 nM) and small (1.25 mM) subunits of polymerase γ and 60 nM DNA template/primer in 50 mM Tris-HCl, 100 mM NaCl, pH 7.8, can be added to a solution containing MgCl₂ (2.5 mM) and various concentrations of nucleotide analogs. Reactions can be quenched and analyzed as described previously. Data can be fit to the same equations as described above.

iii) Assay for Human Polymerase γ 3'S′ Exonuclease Activity: The human polymerase γ exonuclease activity can be studied by measuring the rate of formation of the cleavage products in the absence of dNTP. The reaction can be initiated by adding MgCl₂ (2.5 mM) to a pre-incubated mixture of polymerase γ large subunit (40 nM), small subunit (270 nM), and 1,500 nM chain-terminated template/primer in 50 mM Tris-HCl, 100 mM NaCl, pH 7.8, and quenched with 0.3M EDTA at the designated time points. All reaction mixtures would be analyzed on 20% denaturing polyacrylamide sequencing gels (8M urea), imaged on a Bio-Rad GS-525 molecular image system, and quantified with Molecular Analyst (Bio-Rad). Products formed from the early time points would be plotted as a function of time. Data would be fitted by linear regression with Sigma Plot (Jandel Scientific). The slope of the line can be divided by the active enzyme concentration in the reaction to calculate the kexo for exonuclease activity (see Murakami E, Ray A S, Schinazi R F, Anderson K S. Investigating the effects of stereochemistry on incorporation and removal of 5-fluorocytidine analogs by mitochondrial DNA polymerase gamma: comparison of D- and L-D4FC-TP. Antiviral Res. 2004; 62: 57-64; Feng J Y, Murakami E, Zorca S M, Johnson A A, Johnson K A, Schinazi R F, Furman P A, Anderson K S. Relationship between antiviral activity and host toxicity: comparison of the incorporation efficiencies of 2′,3′-dideoxy-5-fluoro-3′-thiacytidine-triphosphate analogs by human immunodeficiency virus type 1 reverse transcriptase and human mitochondrial DNA polymerase. Antimicrob Agents Chemother. 2004; 48: 1300-6).

### Example 9

### Inhibition of Human DNA Polymerases by NTP's

### Study Objectives

To determine whether a nucleoside-triphosphate analog inhibits human DNA polymerases Alpha, Beta and Gamma and to calculate IC₅₀ values.

### Materials and Methods

Human DNA Polymerase Alpha—Enzyme can be purchased from Chimerx (cat #1075) and assayed based on their recommendations with some modifications. The 2′-Me-UTP was treated with Inorganic Pyrophosphatase (Sigma) to remove any pyrophosphate contamination. A final concentration of 500 μM 2′-Me-UTP can be incubated with 1 mM DTT, 50 mM Tris, 50 mM NaCl, 6 mM MgCl₂, and 1 unit of pyrophosphatase for 1 hour at 37° C. followed by inactivation at 95° C. for 10 minutes. A mixture of 0.05 units of Human DNA Polymerase Alpha and a 5′end radiolabeled 24 nt DNA primer (5′-TCAGGTCCCTGTTCGGGCGCCACT) anneal to a 48 nt DNA template (5′-CAGTGTGGAAAATCTCTAGCAGTGGCGCCCGAACAGGGACCTGAAAGC) can be mixed with increasing concentrations of compound from 0 to 100 μM in 60 mM Tris-HCl (pH 8.0), 5 mM magnesium acetate, 0.3 mg/ml bovine serum albumin, 1 mM dithiothreitol, 0.1 mM spermine, 0.05 mM of each dCTP, dGTP, dTTP, dATP in a final reaction volume of 20 μl for 5 min at 37° C. (all concentrations represent final concentrations after mixing). The reactions can be stopped by mixing with 0.3 M (final) EDTA. Products are separated on a 20% polyacrylamide gel and quantitated on a Bio-Rad Molecular Imager FX. Results from the experiments can be fit to a dose response equation, (y min+((y max)−(y min)))/(1+(compound concentration)/IC₅₀)\hat{ }slope) to determine IC₅₀ values using Graphpad Prism or SynergySoftware Kaleidagraph. Data can be normalized to controls.

Human DNA Polymerase Beta—Enzyme can be purchased from Chimerx (cat #1077) and assayed based on their recommendations with some modifications. A mixture of 0.1 units of Human DNA Polymerase Beta and a 5′end radiolabeled 24 nt DNA primer (5′-TCAGGTCCCTGTTCGGGCGCCACT) anneal to a 48 nt DNA template (5′-CAGTGTGGAAAATCTCTAGCAGTGGCGCCCGAACAGGGACCTGAAAGC) can be mixed with increasing concentrations of compound from 0 to 100 μM in 50 mM Tris-HCl (pH 8.7), 10 mM KCl, 10 mM MgCl₂, 0.4 mg/ml bovine serum albumin, 1 mM dithiothreitol, 15% (v/v) glycerol, and 0.05 mM of each dCTP, dGTP, dTTP, dATP in a final reaction volume of 20 μl for 5 min at 37° C. (all concentrations represent final concentrations after mixing). The reactions can be stopped by mixing with 0.3 M (final) EDTA. Products can be separated on a 20% polyacrylamide gel and quantitated on a Bio-Rad Molecular Imager FX. Results from the experiments can be fit to a dose response equation, (y min+((y max)-(y min)))/(1+(compound concentration)/IC₅₀)\hat{ }slope) to determine IC₅₀ values using Graphpad Prism or SynergySoftware Kaleidagraph. Data can be normalized to controls.

Human DNA Polymerase Gamma—Enzyme can be purchased from Chimerx (cat #1076) and assayed based on their recommendations with some modifications. A mixture of 0.625 units of Human DNA Polymerase Gamma and a 5′end radiolabeled 24 nt DNA primer (5′-TCAGGTCCCTGTTCGGGCGCCACT) anneal to a 36 nt DNA template (5′-TCTCTAGAAGTGGCGCCCGAACAGGGACCTGAAAGC) can be mixed with increasing concentrations of compound from 0 to 100 μM in 50 mM Tris-HCl (pH 7.8), 100 mM NaCl, 5 mM MgCl₂, and 0.05 mM of each dCTP, dGTP, dTTP, dATP in a final reaction volume of 20 μl for 200 min at 37° C. (all concentrations represent final concentrations after mixing). The reactions can be stopped by mixing with 0.3 M (final) EDTA. Products can be separated on a 20% polyacrylamide gel and quantitated on a Bio-Rad Molecular Imager FX. Results from the experiments can be fit to a dose response equation, (y min+((y max)−(y min)))/(1+(compound concentration)/IC₅₀)\hat{ }slope) to determine IC₅₀ values using Graphpad Prism or SynergySoftware Kaleidograph. Data can be normalized to controls.

### Example 10

### Cellular Pharmacology in HepG2 Cells

HepG2 cells are obtained from the American Type Culture Collection (Rockville, MD), and are grown in 225 cm² tissue culture flasks in minimal essential medium supplemented with non-essential amino acids, 1% penicillin-streptomycin. The medium is renewed every three days, and the cells are subcultured once a week. After detachment of the adherent monolayer with a 10 minute exposure to 30 mL of trypsin-EDTA and three consecutive washes with medium, confluent HepG2 cells are seeded at a density of 2.5×10⁶ cells per well in a 6-well plate and exposed to 10 μM of [3H] labeled active compound (500 dpm/pmol) for the specified time periods.

The cells are maintained at 37° C. under a 5% CO₂ atmosphere. At the selected time points, the cells are washed three times with ice-cold phosphate-buffered saline (PBS).

Intracellular active compound and its respective metabolites are extracted by incubating the cell pellet overnight at −20° C. with 60% methanol followed by extraction with an additional 20 pal of cold methanol for one hour in an ice bath. The extracts are then combined, dried under gentle filtered airflow and stored at −20° C. until HPLC analysis.

### Example 11

### Cellular Pharmacology in PBM Cells

Test compounds are incubated in PBM cells at 50 μM for 4 h at 37° C. Then the drug containing media is removed and the PBM cells are washed twice with PBS to remove extracellular drugs. The intracellular drugs are extracted from 10×10⁶ PBM cells using 1 mL 70% ice-cold methanol (containing 10 nM of the internal standard ddATP). Following precipitation, the samples are maintained at room temperature for 15 min followed by vortexing for 30 sec, and then stored 12 h at −20° C. The supernatant is then evaporated to dryness. Dry samples would be stored at −20° C. until LC-MS/MS analysis. Prior to analysis, each sample is reconstituted in 100 μL mobile phase A, and centrifuged at 20,000 g to remove insoluble particulates.

Gradient separation is performed on a Hypersil GOLD column (100×1.0 mm, 3 μm particle size; Thermo Scientific, Waltham, MA, USA). Mobile phase A consists of 2 mM ammonium phosphate and 3 mM hexylamine. Acetonitrile is increased from 10 to 80% in 15 min, and kept at 80% for 3 min. Equilibration at 10% acetonitrile lasts 15 min.

The total run time is 33 min. The flow rate is maintained at 50 μL/min and a 10 μL injection is used. The autosampler and the column compartment are typically maintained at 4.5 and 30° C., respectively.

The first 3.5 min of the analysis is diverted to waste. The mass spectrometer is operated in positive ionization mode with a spray voltage of 3.2 kV.

### Example 12

### Chikungynya Virus Antiviral Activity Assay

Methods for evaluating the efficacy of the compounds described herein against Chikungunya virus, a representative Togaviridae virus, is shown, for example, in Ehteshami, M., Tao, S., Zandi, K., Hsiao, H. M., Jiang, Y., Hammond, E., Amblard, F., Russell, O. O., Mertis, A., and Schinazi, R. F.: Characterization of β-D-N4-hydroxycytidine as a novel inhibitor of chikungunya virus. Antimirob Agents Chemother, 2017 April; 61(4): e02395-16.

Anti-Chikungunya Activity can also be evaluated as outlined in “Anti-Chikungunya Viral Activities of Aplysiatoxin-Related Compounds from the Marine Cyanobacterium Trichodesmium erythraeum” Gupta, D. K.; Kaur, P.; Leong, S. T.; Tan, L. T.; Prinsep, M. R.; Chu, J J. H. Mar Drugs. January 2014; 12(1): 115-127; 10.3390/md12010115 and references cited therein.

### Example 13

### Assaying Compounds for Efficacy Against Mayaro Virus Infection:

A representative assay for determining the efficacy of the compounds described herein against the Mayaro virus, another representative Togaviridae virus, is disclosed in Cavalheiro et al., “Macrophages as target cells for Mayaro virus infection: involvement of reactive oxygen species in the inflammatory response during virus replication,” Anais da Academia Brasileira de Ciências (2016) 88(3): 1485-1499, (Annals of the Brazilian Academy of Sciences). The procedures are summarized below.

### Cell Culture and Virus Propagation

RAW 264.7, a mouse leukaemic macrophage cell line, and J774, a mouse reticulum sarcoma cell line, can be maintained in RPMI-1640 medium (LGC) supplemented with 10% fetal bovine serum (FBS; Invitrogen Life Technologies) in a humidified incubator at 37° C. with 5% CO2. Mouse peritoneal macrophages can be obtained from C57Bl/6 animals by the intraperitoneal injection of 1 mL of sterile 3% thioglycollate. After 96 h, the peritoneal macrophages can be harvested, washed with RPMI and centrifuged at 1,500 rpm for five minutes. Then, the macrophages can be plated at a density of 2×10⁶ cells/well in a 6-well plate with RPMI-1640 supplemented with 10% FBS and incubated at 37° C. with 5% CO2. After 24 h, the plates can be washed with RPMI to remove non-adherent cells before the assays.

MAYV (ATCC VR 66, strain TR 4675) and SINV (AR339) can be propagated in BHK-21 cells grown in α-Minimum Essential Medium (α-MEM; Invitrogen Life Technologies) supplemented with 10% FBS. The cells can be infected with a multiplicity of infection (MOI) of 0.1. After 16 h for SINV and 30 h for MAYV, the culture media can be harvested and cell debris can be removed by centrifugation at 2,000×g for 10 min and the supernatant can be stored at −80° C. Virus stocks titers can be determined by plaque assay in BHK-21 cells.

### Macrophage Infection Assays

Cells can be incubated with MAYV or SINV at a MOI of 1 (for RAW 264.7 and J774) or 5 (for primary peritoneal macrophages), for 1 h at 37° C. in 5% CO2. Then, the medium containing the non-adsorbed virus can be removed, the cells can be washed with serum-free medium and cultured in RPMI supplemented with 5% FBS, at 37° C. in 5% CO2. After the desired periods of infection, conditioned media can be collected for virus titration, LDH assay and cytokine quantification. Cellular extracts can be used for MTT and flow cytometry assays. Virus inactivated by heating at 65° C. for 30 min can be used as control. In some experiments, cells can be treated with 10 mM N-acetyl-L-cysteine (NAC; Sigma-Aldrich) or 50 μM apocynin (Sigma-Aldrich) for 15 h after infection with MAYV.

### Virus Titration by Plaque Assay

BHK-21 cells can be seeded, for example, at a density of 1.25×10⁵ cells per well in 12-wells plates and incubated at 37° C. overnight. Ten-fold serial dilutions of the virus samples can be prepared in α-MEM and incubated with the cells for 1 h at 37° C. (0.2 mL per well). After 1 h adsorption, 2 mL of 1% carboxymethylcellulose (w/v) (Sigma-Aldrich) in α-MEM supplemented with 2% FBS can be layered onto the infected monolayers and the cells can be incubated at 37° C. for 30 h or 48 h, for SINV or MAYV, respectively. Plaques can be visualized by staining the monolayer with 1 mL 1% crystal violet in 20% ethanol.

### Cell Viability Assays

Determination of macrophage viability during infection can be assessed by 3-(4,5-dimethylthiazol-2-yl)-2,5-diphenyltetrazolium bromide (MTT) or lactate dehydrogenase (LDH) release assays. For the MTT assay, cells can be incubated with 0.5 mL 0.5 mg/mL MTT (USB Corporation) in PBS solution for 90 min at 37° C. Then, unreacted dye can be discarded and formazan crystals can be An Acad Bras Cienc (2016) 88 (3) 1488 Mariana G. Cavalheiro et al. solubilized in 0.04 M HCl solution in isopropanol (1 mL per well). The absorbance of samples can be measured at 570 nm and 650 nm for background correction. Lactate dehydrogenase (LDH) release from infected macrophages can be determined by using an LDH detection kit (Promega CytoTox 96 assay kit). The procedures can be performed according to manufacturer's instructions.

### Quantitation of Infected Cells by Flow Cytometry

Flow cytometry analysis can be performed to assess the frequency of MAYV- or SINV-infected cells by detecting intracellular viral antigens. After the desired periods of infection, cells can be washed with PBS, detached by scraping, harvested and fixed in 4% formaldehyde in PBS at room temperature for 15 min. After washing, cells can be permeabilized with 0.1% saponin in PBS and incubated with blocking solution (PBS supplemented with 2% FBS and 0.1% bovine serum albumin) for 20 min, at room temperature. Then, cells can be incubated for 1 h with mouse anti-Eastern Equine Encephalitis virus monoclonal antibody (Chemicon International, Millipore), which reacts with an E1 epitope shared by all alphaviruses. Then, cells can be washed and stained with anti-mouse IgG conjugated to Alexa Fluor 488 (Invitrogen) for 30 min. The percentage of infected cells can be analyzed by FACScan Flow Cytometer and CellQuest software (Becton Dickinson).

### Characterization of Cell Death

Apoptosis/necrosis after infection can be quantified by a double staining method using The Vybrant Apoptosis Assay Kit #2 (Molecular Probes). After the infection period, RAW 264.7 cells can be washed with PBS, detached by scraping, harvested and stained with Annexin V Alexa Fluor 488 (0.5 μg/mL) and propidium iodide (PI, 0.25 μg/mL). To further characterize MAYV-induced cell death, the activity of caspases 3 and 7 can be measured using the Muse™ Caspase-3/7 Kit (Millipore) adapted to flow cytometry. Cells can be washed with PBS, detached by scraping, harvested and incubated with Muse™ Caspase-3/7 Reagent 1:8 and Muse™ Caspase 7-AAD, according to the manufacturer's protocol. For both assays, the percentage of apoptotic and necrotic cells can be analyzed by FACScan Flow Cytometer using the CellQuest software (Bectan Dickinson). UV radiated cells and cells subjected to a freeze-thaw procedure can be used as controls.

### Quantitation of Reactive Oxygen Species (ROS)

The amount of intracellular reactive oxygen species (ROS) can be measured by the formation of the oxidized derivative of 5-(and 6-)-chloromethyl-2′,7′-dichlorodihydrofluorescein diacetate (DCF, Molecular Probes). After 15 h of infection with MAYV, adherent cells can be washed with PBS and incubated with DCF 0.5 μM for 45 minutes. Then, the cells can be washed again, detached by scraping and harvested and analyzed by FACScan Flow Cytometer using the CellQuest software (Bectan Dickinson).

Quantitation of Cytokines The concentrations of cytokines in the conditioned medium of macrophage cultures can be determined by ELISA. TNF concentration can be quantified using the Standard ELISA Development kit (PeproTech), according to the manufacturer's protocol.

### Example 14

### Yellow Fever Virus (YFV) Antiviral Activity Assay: Primary Assay for Antiviral Activity

A monolayer of Human Rhabdomyosarcoma (RD) cells will be grown in 96-well plate in MEM containing 2% inactivated FBS. The tested compounds will be added to the wells in triplicate together with YFV at an MOI=1. The plate will then be incubated at 37° C. with 5% CO₂ for 72 hours. The assay will be conducted in triplicate for each concentration of each compound. After three days, the plate will be viewed under the microscope and the degree of cytopathic effect (CPE) as measure of virus replication inhibition will be expressed as the percent yield of virus control. The results will be evaluated by performing the MTS assay (Promega, WI, USA) according to the manufacturer's protocols. All experiments will be repeated three times independently.

### Focus Forming Unit Reduction Assay (FFURA)

Antiviral activity of each compound will be determined by measuring the reduction in the number of YFV infectious foci in RD cells following treatment with increasing concentrations of each compound. Briefly, infected RD cells which will be treated with different concentrations of each compound will be incubated for 2 days post infection using conditioned-growth medium supplemented with 2% FBS and 1.5% carboxymethyl cellulose (CMC). Antiviral activity of each compound will be determined after visualizing and counting viral foci. The number of YFV foci will be counted using Elispot machine and the virus titer will be expressed as Foci Forming-Unit (FFU). Antiviral activities of the compounds will be determined by calculating the percentage of foci reduction (% RF) against the controls maintained in parallel using the following formula; RF (%)=(C−T)×100/C, where, C is the mean of the number of foci from triplicates treatment without compound added (vehicle control) and T is the mean of the number of foci from triplicates of each treatment measures with the respective compound. Results will represent as the means±standard error of the mean (SEM) from triplicate assay from three independent experiments. Results were confirmed by virus yield reduction assay using quantitative RT-PCR.

### Virus Yield Reduction Assay

Monolayers of RD cells will be prepared in 96-well cell culture microplate and overlaid with YFV (moi=0.1) for 1 hour. After virus adsorption, cells will be washed 3 times with cold sterile PBS to remove unattached viruses and then the cells will be treated for 2 days with increasing concentrations of the tested compounds. After 2 days, the YFV RNA will be extracted from the infected/treated cells and supernatant separately and the yield of YFV will quantified using a one-step specific quantitative RT-PCR for YFV. Nevertheless, the antiviral activity of each nucleoside analogues will be investigated using focus forming unit reduction assay (FFURA) as described previously

### Time-of-Drug-Addition Assay

Confluent monolayers of RD cells will be prepared in 96-well cell culture plate and will be pre-treated with EC₉₀ of each effective drug for 2 h before infection with YFV at MOI=1, concurrently with infection as well as 1, 2, 4 and 6 h post-infection. The cells will then be incubated in the presence of compound for 48 h. At the end of the incubation period, viral load for each time point of treatment will be determined using qRT-PCR as mentioned above.

Statistical analysis

Graph Pad Prism for Windows, Version 5 (Graph Pad Software Inc., San Diego, C A, 2005) will be used to determine the half maximal effective concentration EC₅₀ values and also EC₉₀ of each effective compound. All EC₅₀ and EC₉₀ values will be calculated as the means standard error of the mean (SEM) from triplicate assay from three independent experiments.

### Example 15

### HCV Replicon Assay1

Huh 7 Clone B cells containing HCV Replicon RNA can be seeded in a 96-well plate at 5000 cells/well, and the compounds tested at 10 μM in triplicate immediately after seeding. Following five days incubation (37° C., 5% CO₂), total cellular RNA can be isolated by using versaGene RNA purification kit from Gentra. Replicon RNA and an internal control (TaqMan rRNA control reagents, Applied Biosystems) can be amplified in a single step multiplex Real Time RT-PCR Assay. The antiviral effectiveness of the compounds can be calculated by subtracting the threshold RT-PCR cycle of the test compound from the threshold RT-PCR cycle of the no-drug control (ACt HCV). A ACt of 3.3 equals a 1-log reduction (equal to 90% less starting material) in Replicon RNA levels. The cytotoxicity of the compounds can also be calculated by using the ACt rRNA values. 2′-C-Me-C can be used as the positive control. To determine EC₉₀ and IC₅₀ values, ACt: values can first be first converted into fraction of starting material and then can be used to calculate the % inhibition.

### Example 16

### Efficacy of the Compounds Described Herein Against Dengue

The essential role of a particular viral protein (Dengue virus envelope protein (E)) in viral propogation. Mondotte et al., J. Virol. July 2007, vol. 81 no. 13 7136-7148 discloses an assay useful for identifying compounds for treating infections caused by the Dengue virus, and this assay can be used to identify those compounds described herein which are active against Dengue.

Another assay is described in Levin, 14th International Symposium on Hepatitis C Virus & Related Viruses, Glasgow, UK, 9-13 Sep. 2007. The assay relates to human and Dengue virus polymerase, where putative compounds can be tested against the enzymes, preferably in duplicate, over a range of concentrations, such as from 0.8 mM to 100 mM. The compounds can also be run alongside a control (no inhibitor), a solvent dilution (0.016% to 2% DMSO) and a reference inhibitor.

A suitable high throughput assay for Dengue is described in Lim et al., Antiviral Research, Volume 80, Issue 3, December 2008, Pages 360-369. Dengue virus (DENV) NS5 possesses methyltransferase (MTase) activity at its N-terminal amino acid sequence and is responsible for formation of a type 1 cap structure, m7GpppAm2′-O in the viral genomic RNA. Optimal in vitro conditions for DENV2 2′-O-MTase activity can be characterized using purified recombinant protein and a short biotinylated GTP-capped RNA template. Steady-state kinetics parameters derived from initial velocities can be used to establish a robust scintillation proximity assay for compound testing. Pre-incubation studies by Lim et al., Antiviral Research, Volume 80, Issue 3, December 2008, Pages 360-369, showed that MTase-AdoMet and MTase-RNA complexes can be equally catalytically competent and the enzyme supports a random bi kinetic mechanism. Lim validated the assay with competitive inhibitory agents, S-adenosyl-homocysteine and two homologues, sinefungin and dehydrosinefungin. A GTP-binding pocket present at the N-terminal of DENV2 MTase can be previously postulated to be the cap-binding site. This assay allows rapid and highly sensitive detection of 2′-O-MTase activity, and can be readily adapted for high-throughput screening for inhibitory compounds.

### Example 17

### Anti-Norovirus Activity

Compounds can exhibit anti-norovirus activity by inhibiting norovirus polymerase and/or helicase, by inhibiting other enzymes needed in the replication cycle, or by other pathways.

There is currently no approved pharmaceutical treatment for Norovirus infection (http://www.cdc.gov/ncidod/dvrd/revb/gastro/norovirus-qa.htm), and this has probably at least in part been due to the lack of availability of a cell culture system. Recently, a replicon system has been developed for the original Norwalk G-I strain (Chang, K. O., et al. (2006) Virology 353:463-473).

Both Norovirus replicons and Hepatitis C replicons require viral helicase, protease, and polymerase to be functional in order for replication of the replicon to occur. Most recently, an in vitro cell culture infectivity assay has been reported utilizing Norovirus genogroup I and II inoculums (Straub, T. M. et al. (2007) Emerg. Infect. Dis. 13(3):396-403). This assay is performed in a rotating-wall bioreactor utilizing small intestinal epithelial cells on microcarrier beads. The infectivity assay may be useful for screening entry inhibitors.

### Diagnosis of Norovirus Infection

One can diagnose a norovirus infection by detecting viral RNA in the stools of affected persons, using reverse transcription-polymerase chain reaction (RT-PCR) assays. The virus can be identified from stool specimens taken within 48 to 72 hours after onset of symptoms, although one can obtain satisfactory results using RT-PCR on samples taken as long as 7 days after the onset of symptoms. Other diagnostic methods include electron microscopy and serologic assays for a rise in titer in paired sera collected at least three weeks apart. There are also commercial enzyme-linked immunoassays available, but these tend to have relatively low sensitivity, limiting their use to diagnosis of the etiology of outbreaks. Clinical diagnosis of norovirus infection is often used, particularly when other causative agents of gastroenteritis have been ruled out.

### Example 18

### Determining the Efficacy of the Compounds Against ZIKV and DENV Infection

Material and methods for ZIKV and DENV (serotypes 1-4) infections assays:

Viruses: ZIKV PRVABC59 strain (NCBI accession KU501215) was obtained from the Centers for Diseases Control and Prevention. Virus stocks were generated on C6/36 or Vero cells and viral titers are determined by endpoint titration in Vero (African Green monkey kidney) or human cells, including neuroblastoma (U251), and hepatoblastoma (Huh7). DENV stocks (kindly provided by Dr. Guey Chuen Perng (Emory University & National Cheng Kung University, Taiwan) were generated in Vero or Baby Hamster Kidney cells (BHK) (Clark et al., 2016).

Cytopathic-reduction assay for ZIKV or DENV: For the cytopathic-reduction assay, cells (Vero, U251 or Huh7) are seeded in 96-well plates at 1×10⁴ cells/well and incubated overnight. The next day, culture medium containing 50% cell culture infectious doses of ZIKV or DENV (tested in Vero or BHK cells) are added after which 2-fold serial dilutions of the compounds are added. Cell cytopathic effect (CPE) is measured by MTS readout system (CellTiter 96 AQueous One Solution Proliferation kit, Promega) four (Vero) or five (U251 or Huh7) days after compound addition to determine the levels of ZIKV replication inhibition (Zmurko et al., 2016; Gavegnano et al., 2017). For DENV serotypes 1-4, CPE is measured four to five days after compound addition in Vero or BHK cells.

Focus formation assay: For the focus formation assay (FFA), Vero cells are routinely seeded in 96-well plates at 1.5×10⁴ cells/well and incubated overnight. Next, culture medium containing 70-100 focus forming units of ZIKV or DENV (serotypes 1-4) plus 2-fold serial dilutions of the compounds are added to the cells and incubated for 2 h followed by the addition of overlay methylcellulose medium. Following 2-3 days of incubation, foci are stained using anti-Flavivirus group antigen (4G2, Millipore), followed by HRP-anti-mouse IgG and TrueBlue substrate, and imaged using CTL-Immunospot S6 Micro Analyzer (Priyamvada et al., 2016).

Real-time RT-PCR assay: For the RT-PCR assays, Vero, U251, or Huh7 cells (15,000/well) are seeded in 96-well microplates, and cultured overnight prior to use for infections with ZIKV (MOI=0.001 for Vero or MOI-0.5 for U251 or Huh7) or DENV (with MOI varying from 0.001 to 0.1 for different stocks of serotypes 1-4 for Vero cells). Compounds are added at a dose-dependent manner 1-2 h after ZIKV or DENV. After four days incubation, purified RNA are reverse transcribed into cDNA and amplified in a one-step RT-PCR multiplex reaction with LightCycler 480 RNA Master Hydrolysis Probe (Roche, Indianapolis, IN) using highly conserved sequences complementary to a 76 bp fragment from the ZIKV envelope gene as previously described by Lanciotti (Lanciotti et al., 2008), and an endogenous control (TaqMan Ribosomal RNA Control or beta globin reagents; Applied Biosystems) by using the LightCycler 480 Instrument II (Roche). For detection of dengue viruses, we utilized oligonucleotides primers and probes serotype-specific that rapidly detects all four serotypes in a fourplex RT-PCR assay (Johnoson et al., 2005). For all virological tests, percent inhibition and EC₅₀ value (compound concentration that inhibits viral antigen expression or viral replication by 50%) are determined using CalcuSyn software (Biosoft).

### Combination Studies for ZIKV or DENV.

One goal is to focus on compounds with sub-μM concentrations for hit to lead development, with cell selectivity index (SI)≥100. Hit compounds that demonstrate antiviral potency with no apparent cytotoxicity can be selected for drug-drug combinations with compounds that exhibit different mechanism of action, including viral entry and host inhibitors, among others; These combinations can result in synergistic effects and optimal low doses to rapidly eliminate ZIKV or DENV from infected individuals.

One can use the Chou and Talalay method (Chou & Talalay 1984) for determining synergy, antagonism or additivity (Bassit et al., 2008; Schinazi et al., 2012), particularly with respect to combinations.

### Material and Methods for DENV2 (Serotype 2) Replicon Assay:

Baby hamster kidney (BHK-21) stable cell lines expressing dengue virus serotype 2 [DENV2, New Guinea C strain, Qing et al., 2010)] was kindly provided by Mehul S. Suthar (Emory University).

DENV2 replicon-harboring baby hamster kidney (BHK) cells are exposed to test compounds at concentrations varying from 0.2 to 20 μM to assessment of antiviral activity. Renilla luciferase levels (Promega) are quantified 48 hours after test compounds addition to determine the levels of replication inhibition (EC₅₀, μM).

### REFERENCES

1. Clark, K. B., Hsiao, H. M., Bassit L., Crowe J. E. Jr., Schinazi R. F., Perng G. C., Villinger F. Characterization of dengue virus 2 growth in megakaryocyte-erythrocyte progenitor cells. Virology. 493, 162-72 (2016).
2. Zmurko, J., Marques, R. E., Schols, D., Verbeken, E., Kaptein, S. J. F. & Neyts, J. The Viral Polymerase Inhibitor 7-Deaza-2′-C-Methyladenosine Is a Potent Inhibitor of In Vitro Zika Virus Replication and Delays Disease Progression in a Robust Mouse Infection Model. PLoS Neglected Tropical Diseases 10, e0004695, doi:10.1371/journal.pntd.0004695 (2016).
3. Gavegnano C, Bassit L C, Cox B D, Hsiao H-M, Johnson E L, Suthar M, Chakraborty R, Schinazi R F. Jak inhibitors modulate production of replication-competent Zika Virus in Human Hofbauer, Trophoblasts, and Neuroblastoma cells. Pathogens & immunity. 2, 199-218 (2017).
4. Priyamvada L, Quicke K M, Hudson W H, Onlamoon N, Sewatanon J, Edupuganti S, Pattanapanyasat K, Chokephaibulkit K, Mulligan M J, Wilson P C, Ahmed R, Suthar M S, Wrammert J. Human antibody responses after dengue virus infection are highly cross-reactive to Zika virus. PNAS 113, 7852-7857, (2016).
5. Lanciotti R, Kosoy O, Laven J, Velez J, Lambert A, Johnson A, Stanfield S, Duffy M. Genetic and serologic properties of Zika virus associated with an epidemic, Yap State, Micronesia, 2007. Emerg Infect Dis. 14, 1232-1239 (2008).
6. Johnson B W, Russell B J, Lanciotti R S. Serotype-specific detection of dengue viruses in a fourplex real-time reverse transcriptase PCR assay. J Clin Microbiol 43(10), 4977-4983 (2005).
7. Chou T C, Talalay P. Quantitative analysis of dose-effect relationships: the combined effects of multiple drugs or enzyme inhibitors. Adv Enzyme Regul. 22, 27-55 (1984).
8. Bassit L, Grier J, Bennett M, Schinazi R F. Combinations of 2′-C-methylcytidine analogues with interferon-alpha2b and triple combination with ribavirin in the hepatitis C virus replicon system. Antivir Chem Chemother. 19(1), 25-31 (2008).
9. Schinazi R F, Bassit L, Clayton M M, Sun B, Kohler J J, Obikhod A, Arzumanyan A, Feitelson M A. Evaluation of single and combination therapies with tenofovir disoproxil fumarate and emtricitabine in vitro and in a robust mouse model supporting high levels of hepatitis B virus replication. Antimicrob Agents Chemother. 56(12), 6186-91 (2012).
10. Qing M, Liu W, Yuan Z et al., A high-throughput assay using dengue-1 virus like particles for drug discovery. Antiviral Res. 86(2), 163-71 (2010).

### Example 19

### MERS Assay

### Cells and Virus:

Human lung carcinoma cells (A-549) can be used for the primary antiviral assays and can be obtained from American Type Culture Collection (ATCC, Rockville, Md., USA). The cells can be passed in minimal essential medium (MEM with 0.15% NaCHO₃, Hyclone Laboratories, Logan, Utah, USA) supplemented with 10% fetal bovine serum. When evaluating compounds for efficacy, the serum can be reduced to a final concentration of 2% and the medium can contain gentamicin (Sigma-Aldrich, St. Louis, Mo.) at 50 μg/mL. Since the MERS-Co virus did not produce detectable virus cytopathic effects, virus replication in A549 cells can be detected by titering virus supernatant fluids from infected, compound-treated A549 cells in Vero 76 cells.

Vero 76 cells can be obtained from ATCC and can be routinely passed in MEM with 0.15% NaCHO₃ supplemented with 5% fetal bovine serum. When evaluating compounds, the serum can be reduced to a final concentration of 2% and supplemented with 50 μg/mL of gentamicin.

The Middle Eastern coronavirus strain EMC (MERS-CoV) was an original isolate from humans that was amplified in cell culture by Ron Fouchier (Erasmus Medical Center, Rotterdam, the Netherlands) and was obtained from the Centers for Disease Control (Atlanta, Ga.).

### Controls:

Infergen® (interferon alfacon-1, a recombinant non-naturally occurring type-I interferon (Blatt, L., et al., J. Interferon Cytokine Res. (1996) 16(7):489-499 and Alberti, A., BioDrugs (1999) 12(5):343-357) can be used as the positive control drug in all antiviral assays. Infergen=0.03 ng/mL.

### Antiviral Assay:

Virus can be diluted in MEM to a multiplicity of infection=0.001 and each compound can be diluted in MEM+2% FBS using a half-log 8 dilution series. Compound can be added first to 96 well plates of confluent A549 cells followed within 5 mins by virus. Each test compound dilution can be evaluated for inhibition in triplicate. After plating, the plates can be incubated at 37° C. for 4 d. The plates can then be frozen at −80° C.

### Virus Yield Reduction Assay:

Infectious virus yields from each well from the antiviral assay can be determined. Each plate from an antiviral assays can be thawed. Samples wells at each compound concentration tested can be pooled and titered for infectious virus by CPE assay in Vero 76 cells. The wells can be scored for CPE and virus titers calculated. A 90% reduction in virus yield can then be calculated by regression analysis. This represented a one log₁ 0 inhibition in titer when compared to untreated virus controls.

### Example 20

### VEEV Assay

96-well plates of HeLa-Ohio cells can be prepared and incubated overnight. The plates can be seeded at 4×10⁴ cells per well, which yields 90-100% confluent monolayers in each well after overnight incubation. The test compounds in DMSO can be started at a concentration of 100 μM. 8-fold serial dilutions in MEM medium with 0.1% DMSO, 0% FBS, and 50 μg/mL gentamicin with the test compound concentrations being prepared. To 5 test wells on the 96-well plate can be added 100 μL of each concentration and the plate can be incubated at 37° C.+5% CO₂ for 2 h or 18 h. 3 wells of each dilution with the TC-83 strain Venezuelan equine encephalitis virus (ATCC, stock titer: 10⁶0.8 CCID₅₀/mL) prepared in the medium as described above can be added. 2 wells (uninfected toxicity controls) can be added MEM with no virus. 6 wells can be infected with untreated virus controls. To 6 wells can be added media only as cell controls. A blind, known active compound can be tested in parallel as a positive control. The plate can be incubated at 37° C.+5% CO₂ for 3 d. The plate can be read microscopically for visual CPE and a Neutral red dye plate can also be read using BIO-TEK Instruments INC. EL800. For virus yield reduction assays, the supernatant fluid can be collected from each concentration. The temperature can be held at −80° and each compound can be tested in triplicate. The CC₅₀ can be determined by regression analysis using the CPE of toxicity control wells compared with cell controls. The virus titers can be tested in triplicate using a standard endpoint dilution CCID₅₀ assay and titer calculations can be determined using the Reed-Muench (1948) equation. The concentration of compound required to reduce virus yield by 1 log₁₀ (90%) using regression analysis can be calculated (EC₉₀ value). The concentration of compound required to reduce virus yield by 50% using regression analysis can be calculated (EC₅₀ value).

### Example 21

### Rift Valley Fever Assay

The compounds described herein can be tested for activity against Rift Valley Fever virus using methods known to those skilled in the art (e.g., described in Panchal et al., Antiviral Res. (2012) 93(1):23-29).

### Example 22

Determining the Efficacy of the Compounds against HCoV-OC43 and SARS-CoV-2 Infections

### Viruses

HCoV-OC43 was obtained from ATCC (Manasas, VA) and SARS-CoV-2 was provided by BEI Resources (NR-52281: USA-WA/2020). HCoV-OC43 and SARS-CoV-2 were propagated in Huh-7 and Vero cells, respectively and titrated by TCID₅₀ method followed by storage of aliquots at −80° C. until further use.

### Antiviral Activity Assay Using Virus Yield Assay Method

To determine the best time point for the virus yield assay, a kinetic replication of SARS-CoV-2 and HCoV-OC43 in Vero, Caco2, Calu3 and Huh-7 cells was performed, respectively, and the yield of progeny virus was assessed from the supernatant of viral infected cells at different interval time points using specific q-RT PCR for each virus as mentioned earlier. We determined that 48 and 72 h post-infection were the optimum time point for SARS-CoV-2 and HCoV-OC43, respectively, as there was no observed cell death and cytopathic effect (CPE) on infected cells and more importantly, significant increase in the virus RNA copy number which harvested from the supernatant of the infected cells were observed at that time for SARS-CoV-2 and also HCoV-OC43.

In the next step towards defining the antiviral activity of each compound, we have assessed the antiviral activity of each compound in a dose-dependent manner against SARS-CoV-2 and HCoV-OC43 using a virus yield inhibition assay by determining the viral RNA copy number in collected supernatants, compared to the results from infected but untreated cells, and non-infected and untreated cells as necessary controls. All experiments were performed in triplicate and each experiment repeated three times independently to achieve reliable and statistically meaningful results.

The median effective concentration (EC₅₀) and the concentration with 9000 of inhibitory effect (EC₉₀) were calculated using GraphPad PRISM for Mac, version 7 (GraphPad Software Inc., San Diego, C A, 2005) and reported as the mean±standard deviation (SD).

TABLE 1

| | Anti-SARS-COV-2 activity | |
|---|---|---|
| | in Vero cells (μM) | |
| Structures | EC₅₀ | EC₉₀ |
| 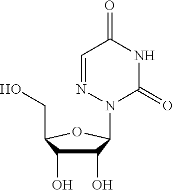 | 0.75 | 2.5 |
| 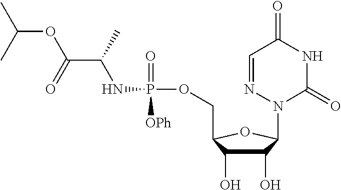 | <10 | |
| 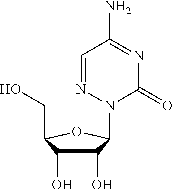 | <10 | |
| 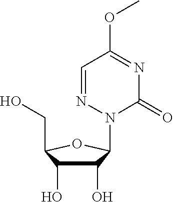 | <10 | |
| 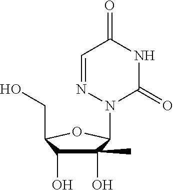 | 10 | |
| 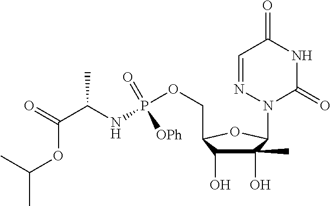 | 10 | |
| 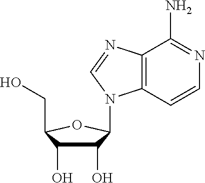 | 6.0 | 15.2 |
| 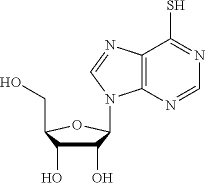 | 11.1 | 23.5 |
| 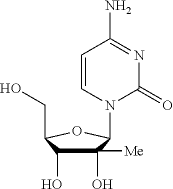 | 9.2 | 29.6 |
| 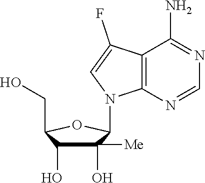 | 7.6 | 28.8 |
| 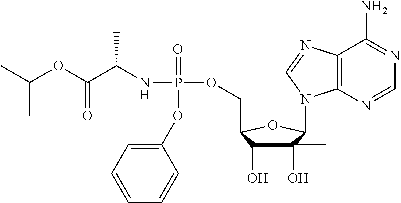 | <10 | |
| 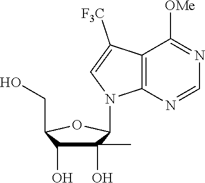 | <10 | |
| 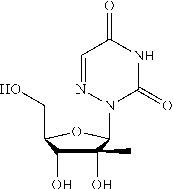 | <10 | |
| 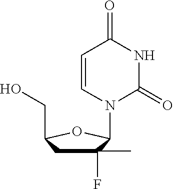 [SMILES: C[C@@]1(F)C[C@@H](CO)O[C@H]1n1ccc(=O)[nH]c1=O] | 10 | |
| 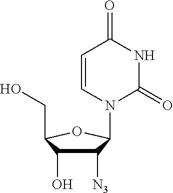 | <10 | |
| 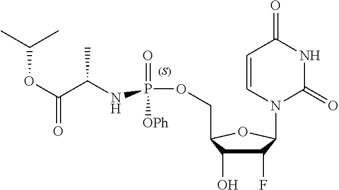 | <10 | |
| 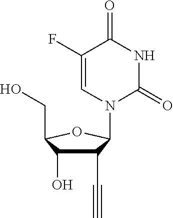 | <10 | |
| 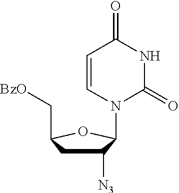 | 10 | |
| 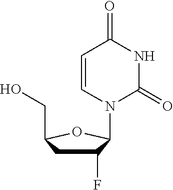 [SMILES: O=c1ccn([C@@H]2O[C@H](CO)C[C@@H]2F)c(=O)[nH]1] | <10 | |
| 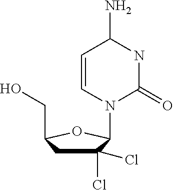 | <10 | |
| 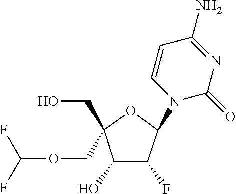 [SMILES: Nc1ccn([C@@H]2O[C@@](CO)(COC(F)F)[C@@H](O)[C@H]2F)c(=O)n1] | <10 | |
| 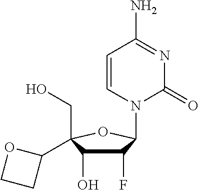 | <10 | |
| 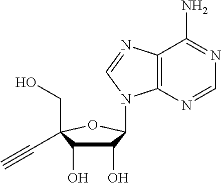 | <10 | |
| 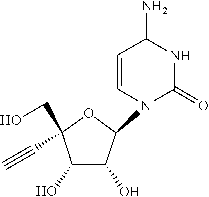 [SMILES: C#C[C@]1(CO)O[C@@H](N2C=CC(N)NC2=O)[C@H](O)[C@@H]1O] | <10 | |
| 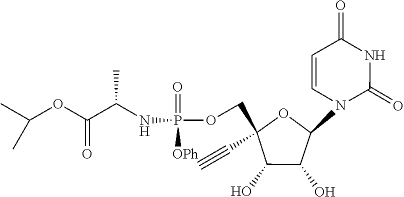 [SMILES: C#C[C@]1(CO[P@@](=O)(N[C@@H](C)C(=O)OC(C)C)Oc2ccccc2)O[C@@H](n2ccc(=O)[nH]c2=O)[C@H](O)[C@@H]1O] | <10 | |
| 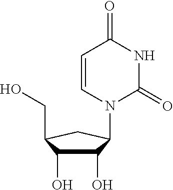 | <10 | |
| 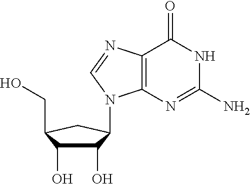 | <10 | |
| 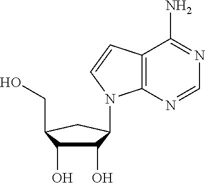 | <10 | |
| 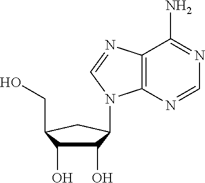 | <10 | |
| 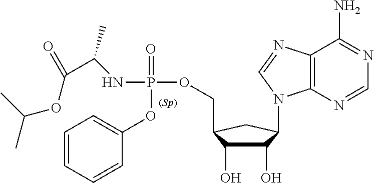 | <10 | |
| 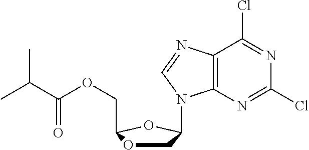 | 2.3 | 6.5 |
| 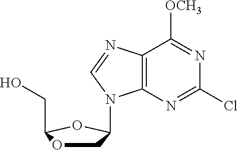 | <10 | |
| 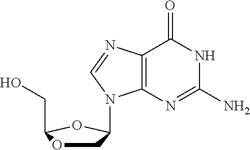 | >10 | |
| 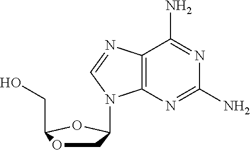 | 10 | |
| 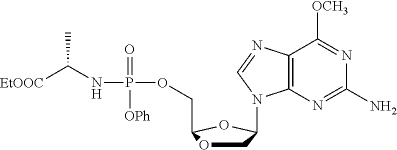 | >10 | |
| 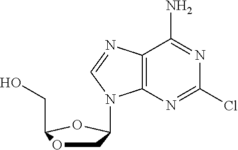 | >10 | |
| 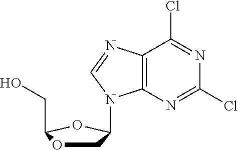 | <10 | |
| 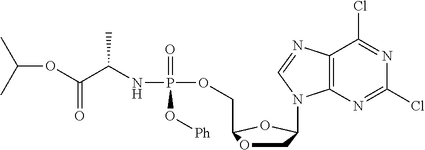 | >10 | |
| 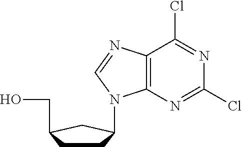 | <10 | |
| 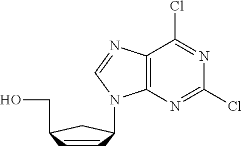 [SMILES: OC[C@@H]1C=C[C@H](n2cnc3c(Cl)nc(Cl)nc32)C1] | <10 | |
| 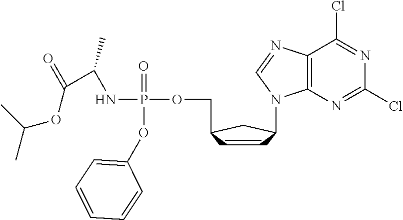 [SMILES: CC(C)OC(=O)[C@H](C)NP(=O)(OC[C@@H]1C=C[C@H](n2cnc3c(Cl)nc(Cl)nc32)C1)Oc1ccccc1] | <10 | |
| 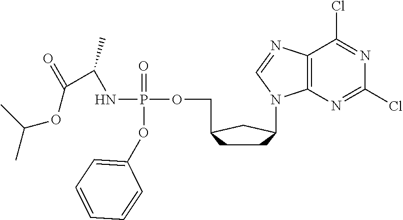 [SMILES: CC(C)OC(=O)[C@H](C)NP(=O)(OC[C@@H]1CC[C@H](n2cnc3c(Cl)nc(Cl)nc32)C1)Oc1ccccc1] | <10 | |
| 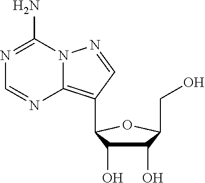 | <10 | |
| 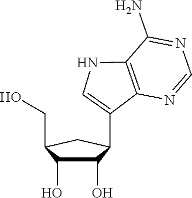 | <10 | |
| 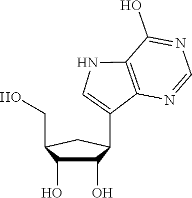 | <10 | |
| 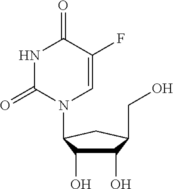 | <10 | |
| 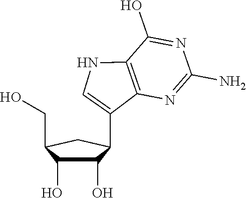 | <10 | |
| 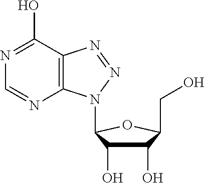 | <10 | |
| 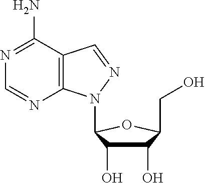 | <10 | |
| 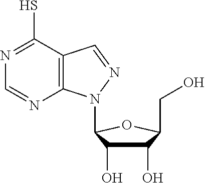 | <10 | |
| 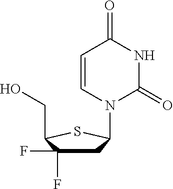 | <10 | |
| 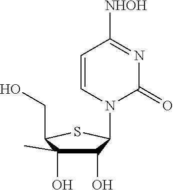 | 1.2 | 8.7 |
| 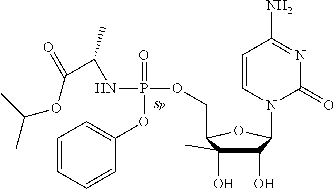 | 9.3 | >10 |
| 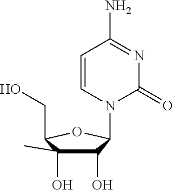 | 0.4 | 3.6 |
| 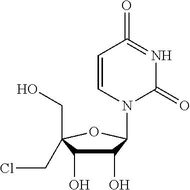 | 2.5 | ≤10 |
| 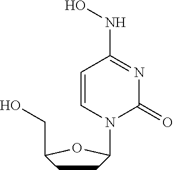 | 2.5 | 83.4 |
| 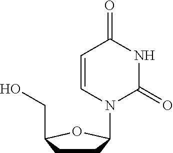 | 10 | |
| 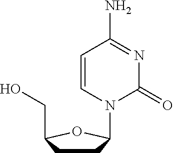 | <10 (95%) | |
| Remdesivir | 1.2 | 3.6 |

### Example 23

Enzymatic Evaluation of SARS-CoV-2 RNA dependent RNA Polymerase Inhibitors

The severe acute respiratory syndrome-coronavirus 2 (SARS-CoV-2) outbreak has caused a global coronavirus (COVID-19) pandemic. The RNA-dependent RNA polymerase (RdRp), also known as nsp12, is a core component of the virus replication and transcription complex and handles the replication and transcription of viral RNA (Yi Jiang, et al., “RNA-dependent RNA polymerase: Structure, mechanism, and drug discovery for COVID-19,” Biochemical and Biophysical Research Communications, Volume 538, 2021, Pages 47-53). RdRp also appears to be a primary target for the antiviral drug remdesivir.

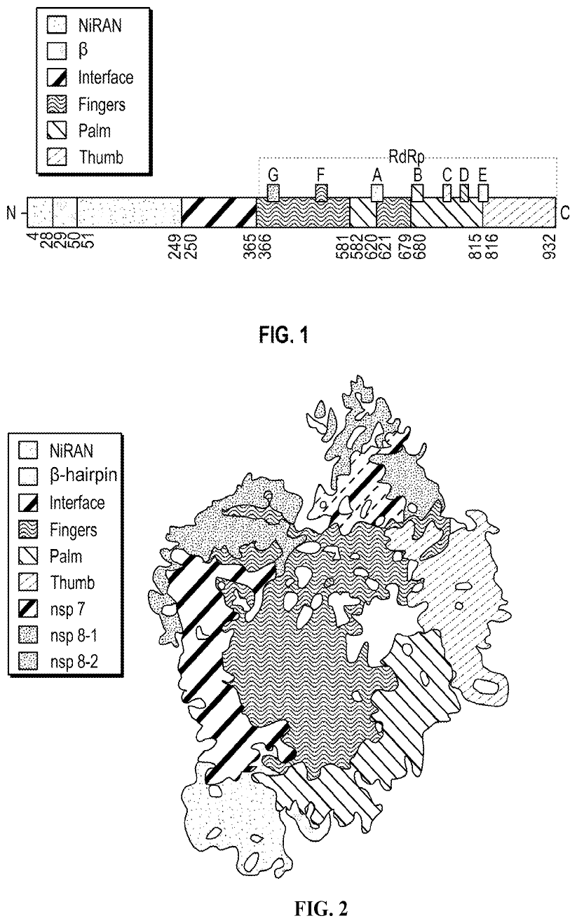

COVID-19 virus nsp12 forms a complex with cofactors nsp7 and nsp8. FIG. 1 shows the structure of the COVID-19 virus nsp12-nsp7-nsp8 complex, including the domain organization of COVID-19 virus nsp12. The interdomain borders are labeled with residue numbers. The N-terminal portion with no cryo-EM map density and the C-terminal residues that cannot be observed in the map are not included in the assignment. The polymerase motifs are colored as follows: motif A, yellow; motif B, red; motif C, green; motif D, violet; motif E, cyan; motif F, blue; and motif G, light brown. This complex is disclosed in Gao et al., “Structure of the RNA-dependent RNA polymerase from COVID-19 virus,” Science 368 (6492), 779-782 (2020).

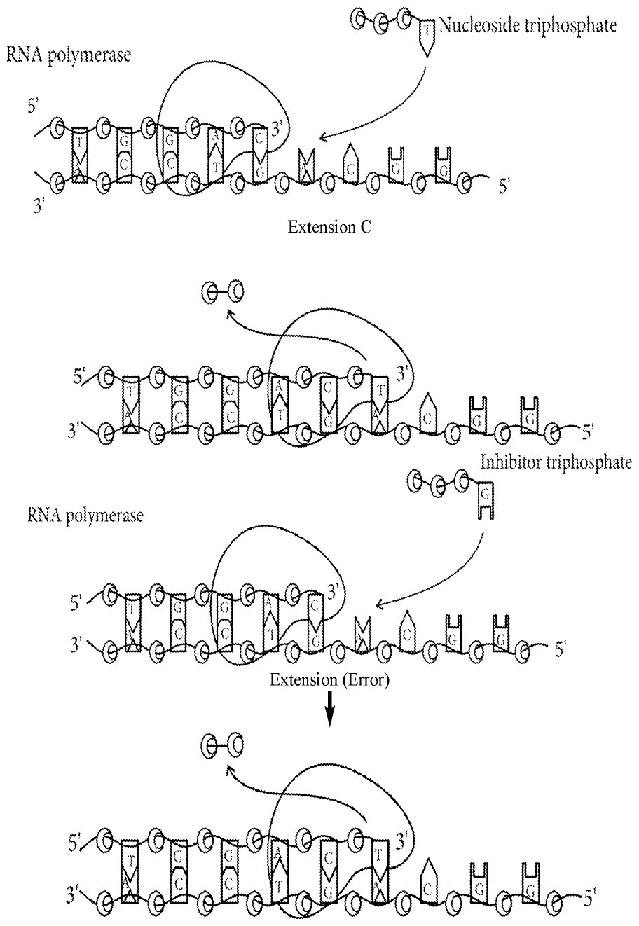

FIG. 2 is a schematic illustration of the structure of the N-terminal NiRAN domain and β hairpin of RdRp. The interacting residues in the palm and fingers subdomain of the RdRp domain and the NiRAN domain are identified by the labels.

FIG. 3 is a schematic illustration showing one embodiment of how an inhibitor triphosphate can interfere with RNA synthesis. An RNA polymerase is an enzyme that synthesizes RNA from a DNA template. As shown in FIG. 3, when a growing RNA chain comes into contact with an RNA polymerase and a naturally-occurring nucleoside triphosphate, the RNA chain is extended. However, when an unnatural inhibitor triphosphate is present, there is an error when the RNA polymerase seeks to add the inhibitor triphosphate to the growing RNA chain.

To measure the ability of modified nucleoside triphosphate inhibitors to disrupt RNA synthesis, a 0.1 μM RdRP complex (reconstituted from three individual nsp proteins) was prepared by mixing the three proteins, then incubating them on ice for 30 minutes.

The complex was buffered using 25 mM Tris-HCl (pH 8). To this buffered solution of the RdRP complex was added 50 μM of a 17-mer RNA primer and 1 μM of a 43-mer RNA template, and the solution was incubated on ice for around 15 minutes.

Once the complex was formed, 0.1 μM hot GTP was added, followed by addition of NTP mixtures (50 mM ATP, CTP and TTP; 25 mM GTP) to provide the nucleoside triphosphates needed for RNA synthesis. Then, either control (water) or an inhibitor triphosphate was added. Two inhibitor triphosphates were evaluated, namely, Remdesivir and

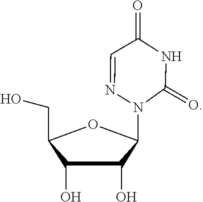

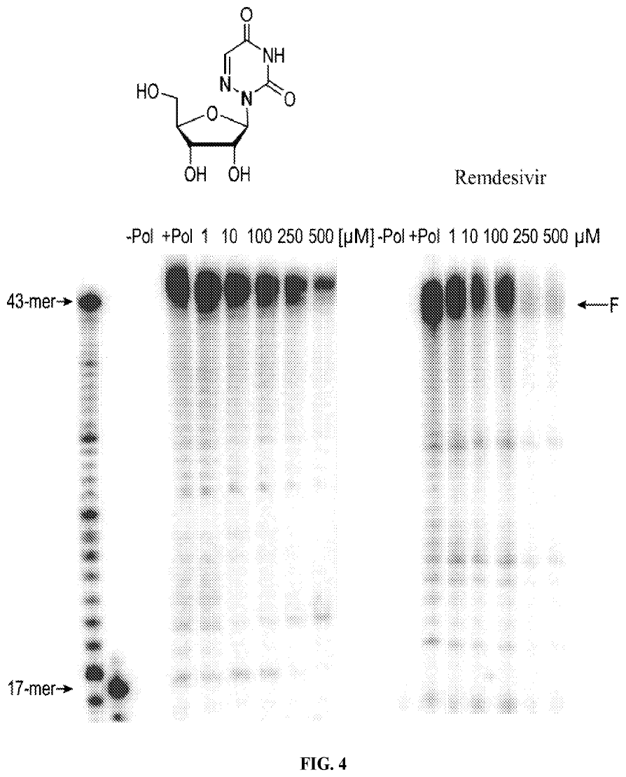

The total reaction mixture, with a volume of around 10 μL, was maintained for 10 mins at 30° C., followed by addition of 5 mM MnCl₂, then the mixture was maintained for 30 more mins at 30° C. The results of polymerase inhibition and/or inhibition of RNA synthesis are shown in FIG. 4.

As shown in FIG. 4, when either

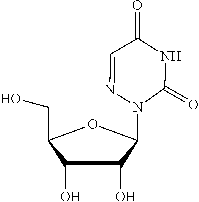

or Remdesivir was added, RNA synthesis was inhibited in a dosage dependent manner. At concentrations of 1, 10 and 100 μM, RNA synthesis was not significantly inhibited. However, significant inhibition was observed at concentrations of 250 and 500 μM. Because the virus does not typically persist for long periods of time, this level of drug concentration can be safely tolerated for the limited periods of time in which it is to be administered.

The present disclosure is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications are cited herein, the disclosures of which are incorporated by reference in their entireties.

We claim:

1. A method for treating or preventing a Coronaviridae, Flaviviridae, Picornaviridae, Bunyaviridae, or Togaviridae infection, comprising administering a treatment or preventative amount of a compound of Formula (A) or Formula (A1) to a patient in need of treatment or prevention thereof: 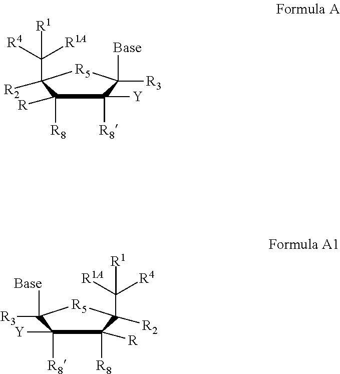
or a pharmaceutically acceptable salt or prodrug thereof, wherein:
Y and R are, independently, selected from the group consisting of H, OH, halo, an optionally substituted O-linked amino acid, substituted or unsubstituted C₁₋₆ alkyl, C₁₋₆ haloalkyl, C₁₋₆ alkoxy, substituted or unsubstituted C₂₋₆ alkenyl, substituted or unsubstituted C₂₋₆ alkynyl, substituted or unsubstituted C₃₋₆ cycloalkyl, cyano, cyanoalkyl, azido, azidoalkyl, OR′, SR′, wherein each R′ is independently a —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, C₁₋₆ alkyl, C₁₋₆ haloalkyl, C₁₋₆ alkoxy, C₂₋₆ alkenyl, C₂₋₆ alkynyl, and C₃₋₆ cycloalkyl, wherein the groups can be substituted with one or more substituents selected from the group consisting of halogen (fluoro, chloro, bromo or iodo), hydroxyl, amino, alkylamino, arylamino, alkoxy, nitro, and cyano,
R¹ is and R^{1A} are, independently, H, CH₃, CH₂F, CHF₂, or CF₃, wherein, when R¹ is Me, the carbon to which it is attached may be wholly or partially R or S or any mixture thereof, or R¹ and R^{1A} can combine to form a C₃₋₇ cycloalkyl ring;
R² is H, CN, N₃, F, CH₂-halogen, CH₂—N₃, O—CH₂—P—(OH)₃, substituted or unsubstituted C₁₋₈alkyl, substituted or unsubstituted C₂₋₈ alkenyl or substituted or unsubstituted C₂₋₈ alkynyl;
R³ is H, substituted or unsubstituted C₁₋₈alkyl, substituted or unsubstituted C₂₋₈ alkenyl, substituted or unsubstituted C₂₋₈ alkynyl, or N₃ when R⁵ is O, and
R³ is selected from the group consisting of H, F, N₃, substituted or unsubstituted (C₁₋₈)alkyl, substituted or unsubstituted (C₂₋₈)alkenyl, substituted or unsubstituted (C₂₋₈)alkynyl, O—(C₁₋₈) alkyl and N₃ when R⁵ is CH₂, Se, CHF, CF₂, —C(CH₃)—, —C(cyclopropyl)-, C═CF₂ or C═CH₂,
R⁵ is O, CH₂, Se, CHF, CF₂, —C(CH₃)—, —C(cyclopropyl)-, C═CF₂ or C═CH₂,
R⁸ and R^{8′} are independently selected from the group consisting of H, OH, halo, an optionally substituted O-linked amino acid, substituted or unsubstituted C₁₋₆ alkyl, C₁₋₆ haloalkyl, C₁₋₆ alkoxy, substituted or unsubstituted C₂₋₆ alkenyl, substituted or unsubstituted C₂₋₆ alkynyl, substituted or unsubstituted C₃₋₆ cycloalkyl, cyano, cyanoalkyl, azido, azidoalkyl, OR′, SR′, wherein each R′ is independently a —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, C₁₋₆ alkyl, C₁₋₆ haloalkyl, C₁₋₆ alkoxy, C₂₋₆ alkenyl, C₂₋₆ alkynyl, C₃₋₆ cycloalkyl, wherein the groups can be substituted with one or more substituents selected from the group consisting of halogen (fluoro, chloro, bromo or iodo), hydroxyl, amino, alkylamino, arylamino, alkoxy, nitro, and cyano,
R⁴ is OH, an optionally substituted O-linked amino acid, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, —O—C(O)O—C₃₋₆ cycloalkyl, OC₁₋₆ alkyl, OC₁₋₆ haloalkyl, OC₁₋₆ alkoxy, OC₂₋₆ alkenyl, OC₂₋₆ alkynyl, OC₃₋₆ cycloalkyl, O—P(O)R⁶R⁷, O—CH₂—P—(OH)₃, O—CH₂—P—(OH)₃, or a mono-, di-, or triphosphate, wherein, when chirality exists at the phosphorous center of R⁴, it may be wholly or partially Rₚ or Sₚ or any mixture thereof,
R⁶ and R⁷ are independently selected from the group consisting of:
(a) OR″ where R″ selected from the group consisting of H, 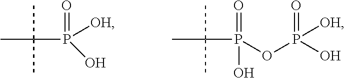
Li, Na, K, substituted or unsubstituted C₁₋₂₀alkyl, substituted or unsubstituted C₃₋₆cycloalkyl, C₁₋₄(alkyl)aryl, benzyl, C₁₋₆ haloalkyl, C₂₋₃(alkyl)OC₁₋₂₀alkyl, aryl, and heteroaryl, such as phenyl and pyridinyl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
where R¹⁶ is independently H, substituted or unsubstituted C₁₋₂₀ alkyl, the carbon chain derived from a fatty alcohol or C₁₋₂₀ alkyl substituted with a C₁₋₆ alkyl, C₁₋₆ alkoxy, di(C₁₋₆ alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆ alkyl, alkoxy, di(C₁₋₆ alkyl)-amino, fluoro, C₃₋₁₀ cycloalkyl, or cycloalkyl;
(b) the ester of a D- or L-amino acid 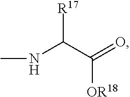
R¹⁷ and R¹⁸ independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl;
Base is selected from the group consisting of: 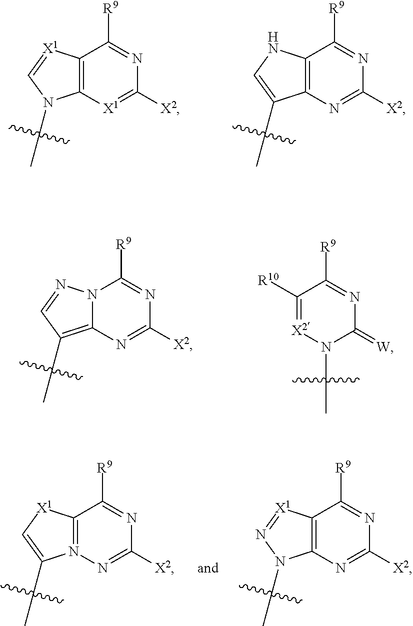
X¹ is CH, C—(C₁₋₆)alkyl, C—(C₂₋₆)alkenyl, C—(C₂₋₆)alkynyl, C—(C₃₋₇)cycloalkyl, C—(C₁₋₆) haloalkyl, C—(C₁₋₆)hydroxyalkyl, C—OR²², C—N(R²²)₂, C-halo, C—CN or N,
X^{1′} is CH, C—(C₁₋₆)alkyl, C—(C₂₋₆)alkenyl, C—(C₂₋₆)alkynyl, C-halo, C—CN or N
R⁹ and X² are independently H, OH, NH₂, halo (i.e., F, Cl, Br, or I), SH, NHOH, O(C₁₋₁₀)alkyl, O(C₂₋₁₀)alkene, O(C₂₋₁₀)alkyne, O(C₃₋₇)cycloalkyl, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, —O—C(O)O—C₃₋₆ cycloalkyl, S(C₁₋₁₀)alkyl, S(C₂₋₁₀)alkene, S(C₂₋₁₀)alkyne, S(C₃₋₇)cycloalkyl, an optionally unsaturated NH(C₁₋₁₀)alkyl, an optionally unsaturated N((C₁₋₁₀)alkyl)₂, NH(C₃₋₇)cycloalkyl, an optionally unsaturated NH(CO)(C₁₋₂₀)alkyl, an optionally unsaturated NH(CO)O(C₁₋₂₀)alkyl, NHOH, an optionally unsaturated NHO(CO)(C₁₋₂₀)alkyl, or an optionally unsaturated NHO(CO)NH(C₁₋₂₀)alkyl, (C₁₋₃)alkyl,
R^{9′} is OH, NH₂, SH, NHOH, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, or —O—C(O)O—C₃₋₆ cycloalkyl,
R¹⁰ is H or F,
X^{2′} is N or CH, and
W is O or S.

2. The method of claim 1, wherein R⁵ is O.

3. The method of claim 2, wherein R² is H or substituted or unsubstituted C₂₋₈ alkynyl.

4. The method of claim 1, wherein R³ is H.

5. The method of claim 1, wherein R¹ is and R^{1A} are H.

6. The method of claim 1, wherein R⁸ and R^{8′} are OH.

7. The method of claim 1, wherein R⁴ is OH or O—P(O)R⁶R⁷.

8. The method of claim 1, wherein Base is

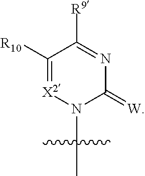

9. The method of claim 8, wherein R^{9′} is OH, NH₂, or NHOH

10. The method of claim 1, wherein Base is

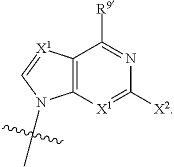

11. The method of claim 10, wherein X² is NH₂, OH or SH.

12. A method for treating or preventing a Coronaviridae, Flaviviridae, Picornaviridae, Bunyaviridae, or Togaviridae infection, comprising administering a treatment or preventative amount of a compound of Formula (B) or (B1) to a patient in need of treatment or prevention thereof: 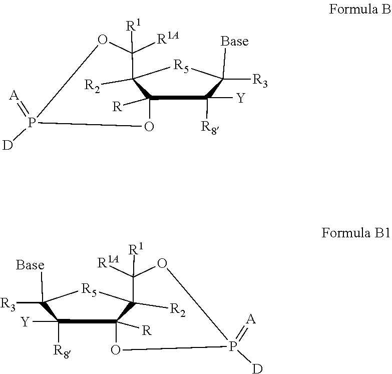
or a pharmaceutically acceptable salt or prodrug thereof, wherein:
Base, Y, R, R¹, R^{1A}, R², R³, R⁵, and R^{8′} are as defined in Formula A,
A is O or S, and
D is selected from the group consisting of:
(a) OR¹⁵ where R¹⁵ is selected from the group consisting of H, substituted or unsubstituted C₁₋₂₀alkyl, substituted or unsubstituted C₃₋₆cycloalkyl, C₁₋₄(alkyl)aryl, benzyl, C₁₋₆haloalkyl, C₂₋₃(alkyl)OC₁₋₂₀ alkyl, aryl, and heteroaryl, such as phenyl and pyridinyl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
(b) the ester of a D- or L-amino acid 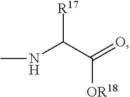
R¹⁷ and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl;
wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl; and
(c) 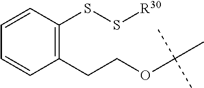
where R³⁰ is selected from the group consisting of substituted or unsubstituted C₁₋₂₀alkyl, substituted or unsubstituted C₃₋₆ cycloalkyl, substituted or unsubstituted (C₂₋₁₀)alkene, substituted or unsubstituted (C₂₋₁₀)alkyne, C₁₋₄(alkyl)aryl, aryl, heteroaryl, and C₁₋₆ haloalkyl.

13. The method of claim 12, wherein R⁵ is O.

14. The method of claim 12, wherein R² is H or substituted or unsubstituted C₂₋₈ alkynyl.

15. The method of claim 12, wherein R³ is H.

16. The method of claim 12, wherein R^{8′} is OH.

17. The method of claim 12, wherein Y is H.

18. The method of claim 12, wherein R¹ and R^{1A} are H.

19. The method of claim 12, wherein A is O.

20. A method for treating or preventing a Coronaviridae, Flaviviridae, Picornaviridae, Bunyaviridae, or Togaviridae infection, comprising administering a treatment or preventative amount of a compound of Formula (C) or (C1) to a patient in need of treatment or prevention thereof: 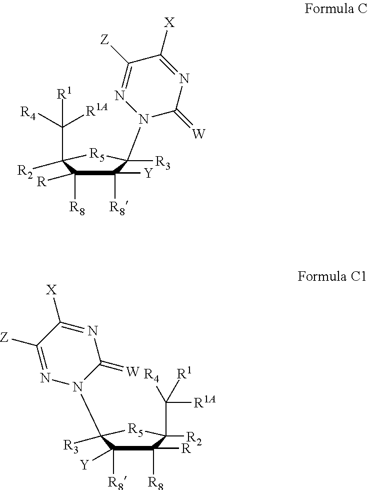
or a pharmaceutically acceptable salt or prodrug thereof, wherein:
R, R¹, R^{1A}, R², R³, R⁵, R⁸, R^{8′} and Y are as defined in Formula A,
X is OH, NH₂, SH, NHOH, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, or —O—C(O)O—C₃₋₆ cycloalkyl,
Z is H or F, and
W is O or S.

21. The method of claim 20, wherein R⁵ is O.

22. The method of claim 20, wherein R² is H or substituted or unsubstituted C₂₋₈ alkynyl.

23. The method of claim 20, wherein R³ is H.

24. The method of claim 20, wherein R⁸ and R^{8′} are OH.

25. The method of claim 20, wherein Y is H.

26. The method of claim 20, wherein R is H.

27. The method of claim 20, wherein Z is H.

28. The method of claim 20, wherein X is OH, NH₂ or NHOH.

29. The method of claim 20, wherein W is O.

30. The method of claim 20, wherein R¹ and R^{1A} are H.

31. The method of claim 20, wherein R⁴ is OH or O—P(O)R⁶R⁷.

32. A method for treating or preventing a Coronaviridae, Flaviviridae, Picornaviridae, Bunyaviridae, or Togaviridae infection, comprising administering a treatment or preventative amount of a compound of Formula (D) or (D1) to a patient in need of treatment or prevention thereof: 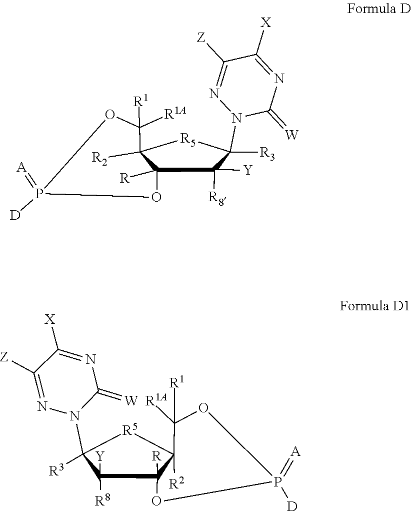
or a pharmaceutically acceptable salt or prodrug thereof, wherein R, R¹, R^{1A}, R², R³, R⁵, R^{8′} and Y are as defined in Formula A, and A and D are as defined in Formula C.

33. The method of claim 32, wherein R⁵ is O.

34. The method of claim 32, wherein R² is H or substituted or unsubstituted C₂₋₈ alkynyl.

35. The method of claim 32, wherein R³ is H.

36. The method of claim 32, wherein R^{8′} is OH.

37. The method of claim 32, wherein Y is H.

38. The method of claim 32, wherein R is H.

39. The method of claim 32, wherein Z is H.

40. The method of claim 32, wherein X is OH, NH₂ or NHOH.

41. The method of claim 32, wherein W is O.

42. The method of claim 32, wherein R¹ and R^{1A} are H.

43. The method of claim 32, wherein R⁴ is OH or O—P(O)R⁶R⁷.

44. A method for treating or preventing a Coronaviridae, Flaviviridae, Picornaviridae, Bunyaviridae, or Togaviridae infection, comprising administering a treatment or preventative amount of a compound of Formula (E) or (E1) to a patient in need of treatment or prevention thereof: 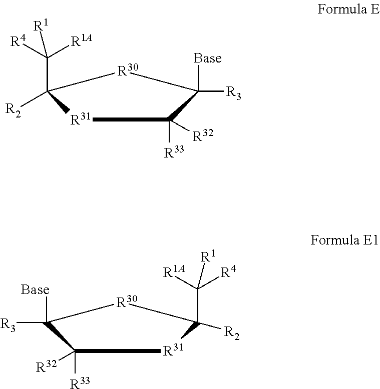
or a pharmaceutically acceptable salt or prodrug thereof, wherein:
Base, R¹, R^{1A}, R², R³, and R⁴ are as defined in Formula A,
R³⁰ is O or CH₂,
R³¹ is O or S,
R³¹ is O when R³⁰ is S, and
R³² and R³³ are independently H, F, C₁-C₃ alkyl, C₂-C₃ alkene, or C₂-C₃ alkyne.

45. The method of claim 44, wherein R³⁰ is O.

46. The method of claim 44, wherein R³¹ is O.

47. The method of claim 44, wherein R³² and R³³ are, independently, H or F.

48. The method of claim 44, wherein R³ is H.

49. The method of claim 44, wherein R² is N₃ or substituted or unsubstituted C₂₋₈ alkynyl.

50. The method of claim 44, wherein R¹ and R^{1A} are H.

51. The method of claim 44, wherein R⁴ is OH or O—P(O)R⁶R⁷.

52. The method of claim 44, wherein Base is

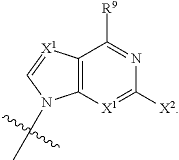

53. The method of claim 52, wherein X¹ is N.

54. A method for treating or preventing a Coronaviridae, Flaviviridae, Picornaviridae, Bunyaviridae, or Togaviridae infection, comprising administering a treatment or preventative amount of a compound of Formula (F) or (F1) to a patient in need of treatment or prevention thereof: 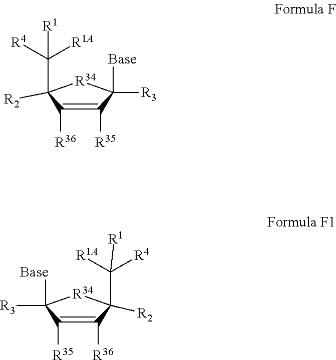
or a pharmaceutically acceptable salt or prodrug thereof, wherein:
Base, R¹, R^{1A}, R², R³, and R⁴ are as defined in Formula A,
R³⁴ is O or CH₂, and
R³⁵ and R³⁶ are independently H, F or CH₃.

55. The method of claim 54, wherein R³⁵ and R³⁶ are H.

56. The method of claim 54, wherein R³⁴ is CH₂.

57. The method of claim 54, wherein R⁴ is OH or O—P(O)R⁶R⁷.

58. The method of claim 54, wherein R³ is H.

59. The method of claim 54, wherein R² is H or substituted or unsubstituted C₂₋₈ alkynyl.

60. The method of claim 54, wherein R¹ and R^{1A} are H.

61. A method for treating or preventing a Coronaviridae, Flaviviridae, Picornaviridae, Bunyaviridae, or Togaviridae infection, comprising administering a treatment or preventative amount of a compound having one of the following formulas to a patient in need of treatment or prevention thereof: 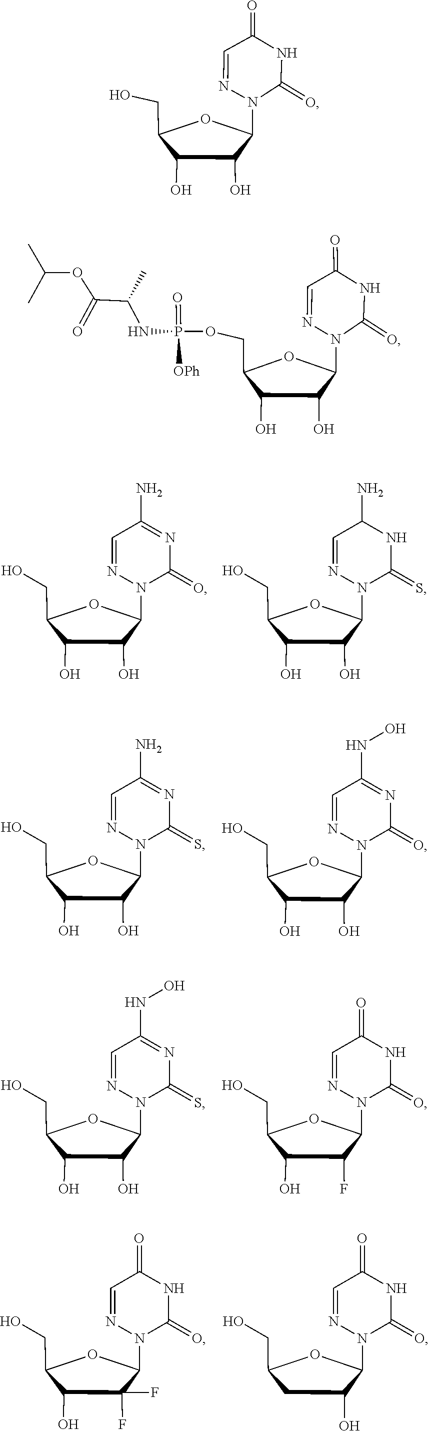 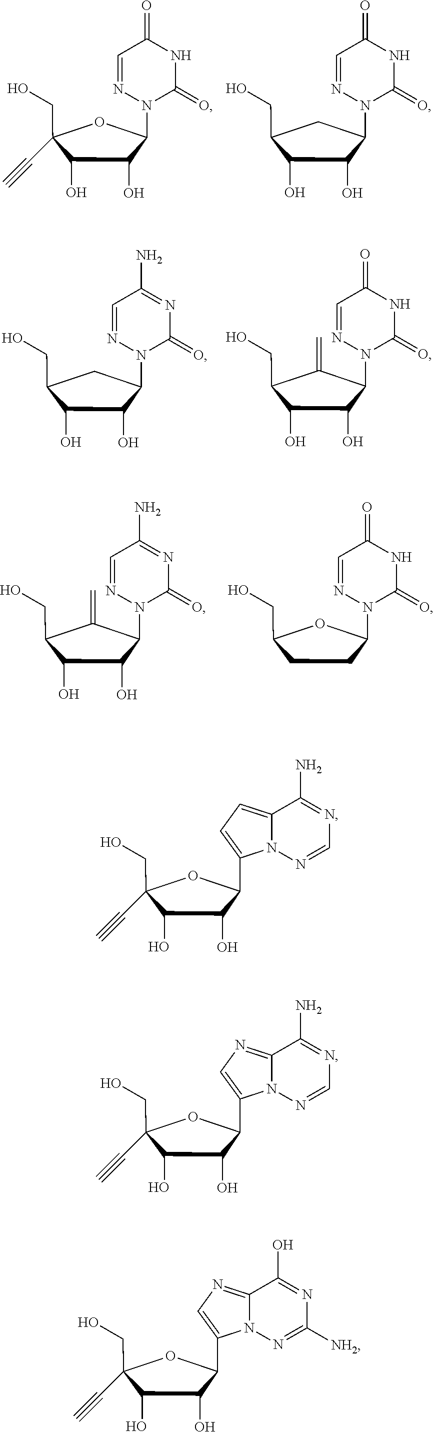 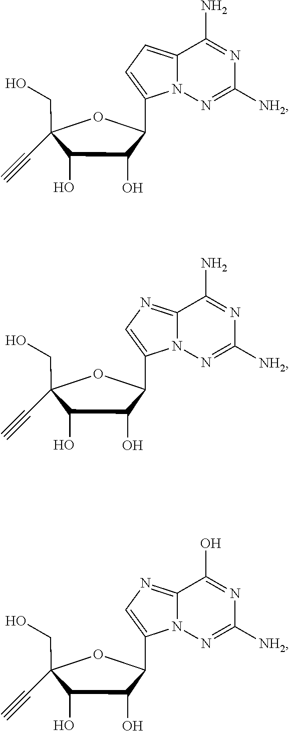 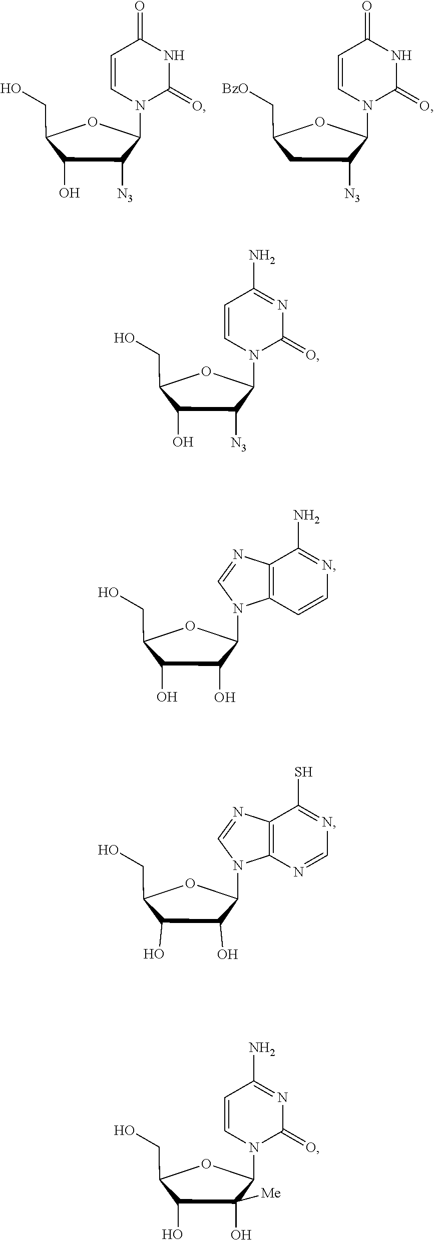 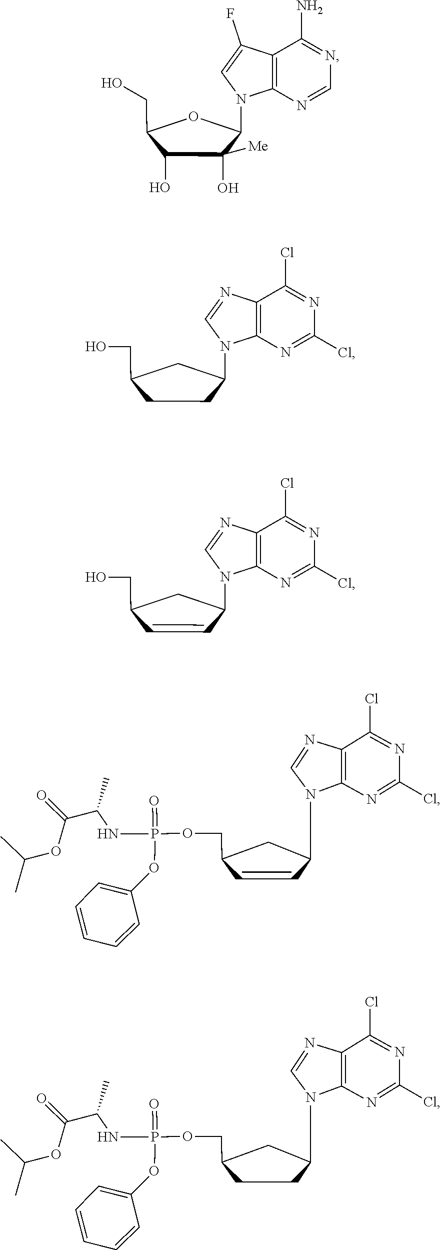 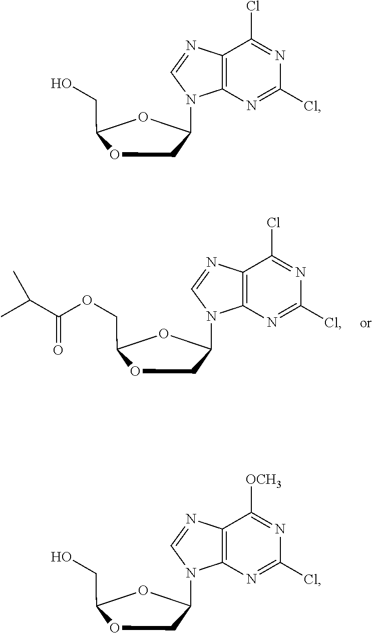
or a pharmaceutically acceptable salt or prodrug thereof.

62. A method for treating or preventing a Coronaviridae, Flaviviridae, Picornaviridae, Bunyaviridae, or Togaviridae infection, comprising administering a treatment or preventative amount of a compound of one of the following formulas to a patient in need of treatment or prevention thereof: 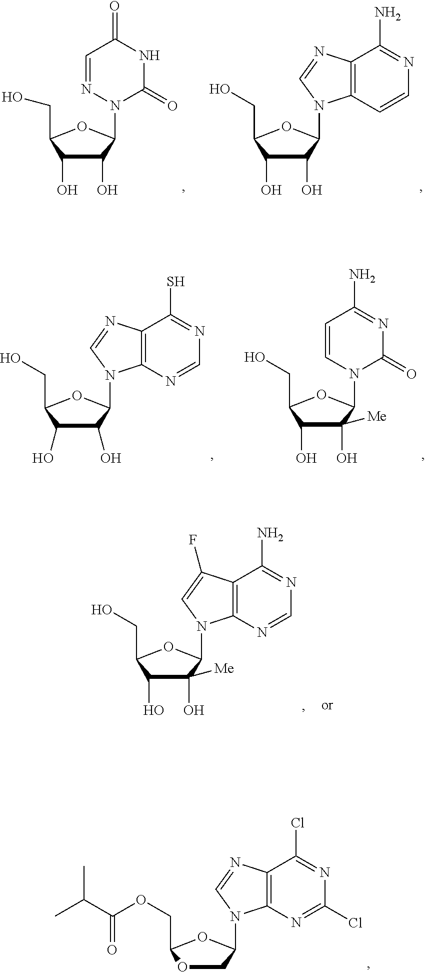
or a pharmaceutically-acceptable salt or prodrug thereof.

63. A method for treating or preventing a Coronaviridae, Flaviviridae, Picornaviridae, Bunyaviridae, or Togaviridae infection, comprising administering a treatment or preventative amount of a compound of one of the following formulas to a patient in need of treatment or prevention thereof: 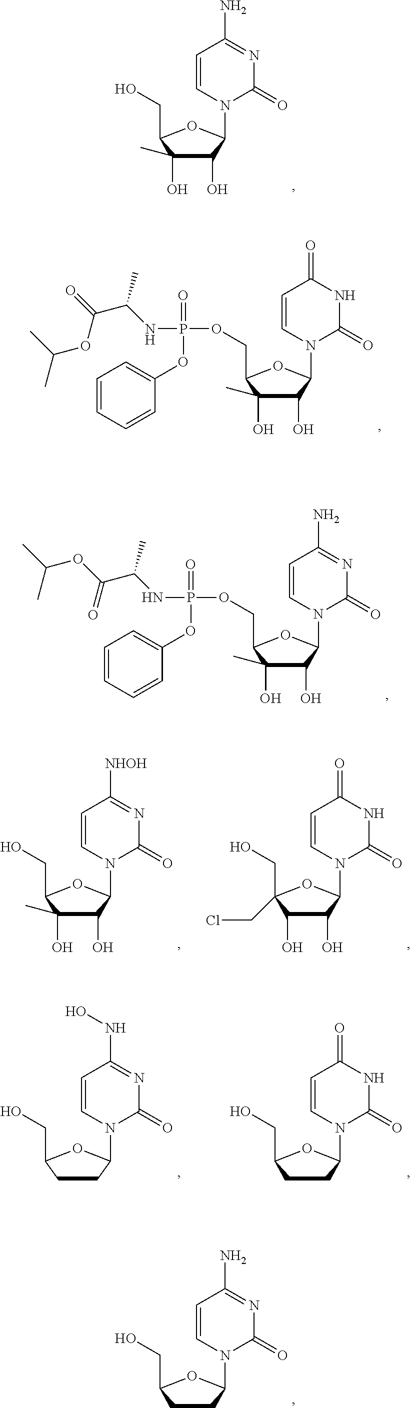
or a pharmaceutically-acceptable salt or prodrug thereof.

64. The method of any of claims 1-63, wherein the compounds can be present in the β-D or β-L configuration.

65. The method of any of claims 1-63, wherein the virus is a Coronavirus.

66. The method of claim 64, wherein the Coronavirus is SARS-CoV2, MERS, SARS, or OC-43.

67. The method of claim 66, wherein the Coronavirus is SARS-CoV2.

68. The method of any of claims 1-63, wherein the compound is co-administered with one or more additional active compounds selected from the group consisting of fusion inhibitors, entry inhibitors, protease inhibitors, polymerase inhibitors, antiviral nucleosides, viral entry inhibitors, viral maturation inhibitors, JAK inhibitors, angiotensin-converting enzyme 2 (ACE2) inhibitors, SARS-CoV-specific human monoclonal antibodies, including CR3022, and agents of distinct or unknown mechanism.

69. The method of claim 68, wherein the compound is administered with remdesivir, N-hydroxy cytidine, or a pharmaceutically-acceptable salt or prodrug thereof.

70. The method of claim 68, wherein the additional active compound is a JAK inhibitor, and the JAK inhibitor is Jakafi, Tofacitinib, or Baricitinib, or a pharmaceutically-acceptable salt or prodrug thereof.

71. The method of claim 68, wherein the one or more additional active agents comprise an anticoagulant or a platelet aggregation inhibitor.

72. The method of claim 68, wherein the one or more additional active agents comprise an ACE-2 inhibitor, a CYP-450 inhibitor, or NOX inhibitor.

73. The use of a compound of any of claims 1-63 in the preparation of a medicament for use in treating or preventing a Coronaviridae, Flaviviridae, Picornaviridae, Bunyaviridae, or Togaviridae infection.

74. The use of claim 73, wherein the infection is a Coronaviridae infection.

75. The use of claim 74, wherein the Coronavirus is SARS-CoV2, MERS, SARS, or OC-43.

76. The use of claim 74, wherein the Coronavirus is SARS-CoV2.

77. The use of claim 73, wherein the medicament further comprises one or more additional active compounds selected from the group consisting of fusion inhibitors, entry inhibitors, protease inhibitors, polymerase inhibitors, antiviral nucleosides, viral entry inhibitors, viral maturation inhibitors, JAK inhibitors, angiotensin-converting enzyme 2 (ACE2) inhibitors, SARS-CoV-specific human monoclonal antibodies, including CR3022, and agents of distinct or unknown mechanism.

78. The use of claim 73, wherein the medicament further comprises remdesivir, N-hydroxy cytidine, or a pharmaceutically-acceptable salt or prodrug thereof.

79. The use of claim 73, wherein the medicament further comprises a JAK inhibitor, and the JAK inhibitor is Jakafi, Tofacitinib, or Baricitinib, or a pharmaceutically-acceptable salt or prodrug thereof.

80. The use of claim 73, wherein the medicament further comprises an anticoagulant or a platelet aggregation inhibitor.

81. The use of claim 73, wherein the medicament further comprises an ACE-2 inhibitor, a CYP-450 inhibitor, or a NOX inhibitor.

82. The use of claim 73, wherein the medicament is is a transdermal composition or a nanoparticulate composition.

83. A method for treating or preventing a Coronaviridae, Flaviviridae, Picornaviridae, Bunyaviridae, or Togaviridae infection, comprising administering a treatment or preventative amount of a compound of Formula (A) or Formula (A1) to a patient in need of treatment or prevention thereof: 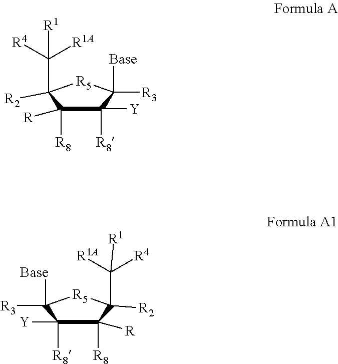
or a pharmaceutically acceptable salt or prodrug thereof, wherein:
Y and R are, independently, selected from the group consisting of H, OH, halo, an optionally substituted O-linked amino acid, substituted or unsubstituted C₁₋₆ alkyl, C₁₋₆ haloalkyl, C₁₋₆ alkoxy, substituted or unsubstituted C₂₋₆ alkenyl, substituted or unsubstituted C₂₋₆ alkynyl, substituted or unsubstituted C₃₋₆ cycloalkyl, cyano, cyanoalkyl, azido, azidoalkyl, OR′, SR′, wherein each R′ is independently a —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, C₁₋₆ alkyl, C₁₋₆ haloalkyl, C₁₋₆ alkoxy, C₂₋₆ alkenyl, C₂₋₆ alkynyl, and C₃₋₆ cycloalkyl, wherein the groups can be substituted with one or more substituents selected from the group consisting of halogen (fluoro, chloro, bromo or iodo), hydroxyl, amino, alkylamino, arylamino, alkoxy, nitro, and cyano,
R¹ is and R^{1A} are, independently, H, CH₃, CH₂F, CHF₂, or CF₃, wherein, when R¹ is Me, the carbon to which it is attached may be wholly or partially R or S or any mixture thereof, or R¹ and R^{1A} can combine to form a C₃₋₇ cycloalkyl ring;
R² is H, CN, N₃, F, CH₂-halogen, CH₂—N₃, O—CH₂—P—(OH)₃, substituted or unsubstituted C₁₋₈alkyl, substituted or unsubstituted C₂₋₈ alkenyl or substituted or unsubstituted C₂₋₈ alkynyl;
R³ is H, substituted or unsubstituted C₁₋₈ alkyl, substituted or unsubstituted C₂₋₈ alkenyl, substituted or unsubstituted C₂₋₈ alkynyl, or N₃ when R⁵ is O, and R³ is selected from the group consisting of H, F, N₃, substituted or unsubstituted (C₁₋₈)alkyl, substituted or unsubstituted (C₂₋₈)alkenyl, substituted or unsubstituted (C₂₋₈)alkynyl, O—(C₁₋₈) alkyl and N₃ when R⁵ is CH₂, Se, CHF, CF₂, —C(CH₃)—, —C(cyclopropyl)-, C═CF₂ or C═CH₂,
R⁵ is O, CH₂, Se, CHF, CF₂, —C(CH₃)—, —C(cyclopropyl)-, C═CF₂ or C═CH₂,
R⁸ and R^{8′} are independently selected from the group consisting of H, OH, halo, an optionally substituted O-linked amino acid, substituted or unsubstituted C₁₋₆ alkyl, C₁₋₆ haloalkyl, C₁₋₆ alkoxy, substituted or unsubstituted C₂₋₆ alkenyl, substituted or unsubstituted C₂₋₆ alkynyl, substituted or unsubstituted C₃₋₆ cycloalkyl, cyano, cyanoalkyl, azido, azidoalkyl, OR′, SR′, wherein each R′ is independently a —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, C₁₋₆ alkyl, C₁₋₆ haloalkyl, C₁₋₆ alkoxy, C₂₋₆ alkenyl, C₂₋₆ alkynyl, C₃₋₆ cycloalkyl, wherein the groups can be substituted with one or more substituents selected from the group consisting of halogen (fluoro, chloro, bromo or iodo), hydroxyl, amino, alkylamino, arylamino, alkoxy, nitro, and cyano,
R⁴ is OH, an optionally substituted O-linked amino acid, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, —O—C(O)O—C₃₋₆ cycloalkyl, OC₁₋₆ alkyl, OC₁₋₆ haloalkyl, OC₁₋₆ alkoxy, OC₂₋₆ alkenyl, OC₂₋₆ alkynyl, OC₃₋₆ cycloalkyl, O—P(O)R⁶R⁷, O—CH₂—P—(OH)₃, O—CH₂—P—(OH)₃, or a mono-, di-, or triphosphate, wherein, when chirality exists at the phosphorous center of R⁴, it may be wholly or partially Rₚ or Sₚ or any mixture thereof,
R⁶ and R⁷ are independently selected from the group consisting of:
(a) OR¹⁵ where R¹⁵ selected from the group consisting of H, 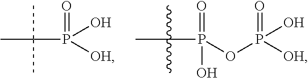
Li, Na, K, substituted or unsubstituted C₁₋₂₀alkyl, substituted or unsubstituted C₃₋₆cycloalkyl, C₁₋₄(alkyl)aryl, benzyl, C₁₋₆ haloalkyl, C₂₋₃(alkyl)OC₁₋₂₀alkyl, aryl, and heteroaryl, such as phenyl and pyridinyl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
where R¹⁶ is independently H, substituted or unsubstituted C₁₋₂₀ alkyl, the carbon chain derived from a fatty alcohol or C₁₋₂₀ alkyl substituted with a C₁₋₆ alkyl, C₁₋₆ alkoxy, di(C₁₋₆ alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆ alkyl, alkoxy, di(C₁₋₆ alkyl)-amino, fluoro, C₃₋₁₀ cycloalkyl, or cycloalkyl;
(b) the ester of a D- or L-amino acid 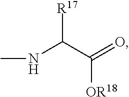
R¹⁷ and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl;
Base is 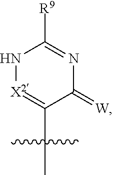
X¹ is CH, C—(C₁₋₆)alkyl, C—(C₂₋₆)alkenyl, C—(C₂₋₆)alkynyl, C—(C₃₋₇)cycloalkyl, C—(C₁₋₆) haloalkyl, C—(C₁₋₆)hydroxyalkyl, C—OR²², C—N(R²²)₂, C-halo, C—CN or N,
X^{1′} is CH, C—(C₁₋₆)alkyl, C—(C₂₋₆)alkenyl, C—(C₂₋₆)alkynyl, C-halo, C—CN or N
R⁹ and X² are independently H, OH, NH₂, halo (i.e., F, Cl, Br, or I), SH, NHOH, O(C₁₋₁₀)alkyl, O(C₂₋₁₀)alkene, O(C₂₋₁₀)alkyne, O(C₃₋₇)cycloalkyl, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, —O—C(O)O—C₃₋₆ cycloalkyl, S(C₁₋₁₀)alkyl, S(C₂₋₁₀)alkene, S(C₂₋₁₀)alkyne, S(C₃₋₇)cycloalkyl, an optionally unsaturated NH(C₁₋₁₀)alkyl, an optionally unsaturated N((C₁₋₁₀)alkyl)₂, NH(C₃₋₇)cycloalkyl, an optionally unsaturated NH(CO)(C₁₋₂₀)alkyl, an optionally unsaturated NH(CO)O(C₁₋₂₀)alkyl, NHOH, an optionally unsaturated NHO(CO)(C₁₋₂₀)alkyl, or an optionally unsaturated NHO(CO)NH(C₁₋₂₀)alkyl, (C₁₋₃)alkyl,
R^{9′} is OH, NH₂, SH, NHOH, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, or —O—C(O)O—C₃₋₆ cycloalkyl,
R¹⁰ is H or F,
X^{2′} is N or CH, and
W is O or S.

84. The method of claim 83, wherein R⁵ is O, R² is H or substituted or unsubstituted C₂₋₈ alkynyl, R³ is H, R¹ is and R^{1A} are H, R⁸ and R^{8′} are OH, R⁴ is OH or O—P(O)R⁶R⁷, R^{9′} is OH, NH₂, or NHOH, and/or X² is NH₂, OH or SH.

85. A method for treating or preventing a Coronaviridae, Flaviviridae, Picornaviridae, Bunyaviridae, or Togaviridae infection, comprising administering a treatment or preventative amount of a compound of Formula (B) or (B1) to a patient in need of treatment or prevention thereof: 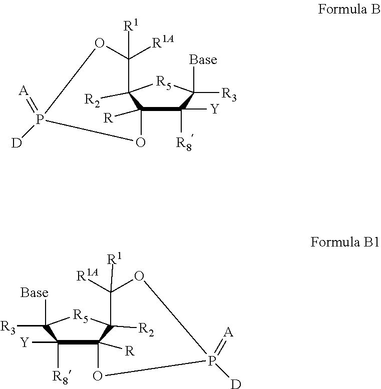
or a pharmaceutically acceptable salt or prodrug thereof, wherein:
Base, Y, R, R¹, R^{1A}, R², R³, R⁵, and R^{8′} are as defined in Formula A,
A is O or S, and
D is selected from the group consisting of:
(a) OR¹⁵ where R¹⁵ is selected from the group consisting of H, substituted or unsubstituted C₁₋₂₀alkyl, substituted or unsubstituted C₃₋₆cycloalkyl, C₁₋₄(alkyl)aryl, benzyl, C₁₋₆haloalkyl, C₂₋₃(alkyl)OC₁₋₂₀ alkyl, aryl, and heteroaryl, such as phenyl and pyridinyl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
(b) the ester of a D- or L-amino acid 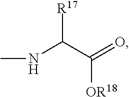
R¹⁷ and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl; and
(c) 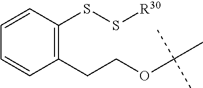
where R³⁰ is selected from the group consisting of substituted or unsubstituted C₁₋₂₀alkyl, substituted or unsubstituted C₃₋₆ cycloalkyl, substituted or unsubstituted (C₂₋₁₀)alkene, substituted or unsubstituted (C₂₋₁₀)alkyne, C₁₋₄(alkyl)aryl, aryl, heteroaryl, and C₁₋₆ haloalkyl.

86. The method of claim 85, wherein R⁵ is O, R² is H or substituted or unsubstituted C₂₋₈ alkynyl, R³ is H, R^{8′} is OH, Y is H, R¹ and R^{1A} are H, and/or A is O.

87. A method for treating or preventing a Coronaviridae, Flaviviridae, Picornaviridae, Bunyaviridae, or Togaviridae infection, comprising administering a treatment or preventative amount of a compound of Formula (C) or (C1) to a patient in need of treatment or prevention thereof: 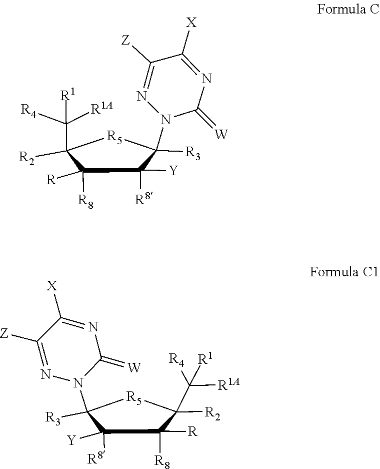
or a pharmaceutically acceptable salt or prodrug thereof, wherein:
R, R¹, R^{1A}, R², R³, R⁵, R⁸, R^{8′} and Y are as defined in Formula A,
X is OH, NH₂, SH, NHOH, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, or —O—C(O)O—C₃₋₆ cycloalkyl,
Z is H or F, and
W is O or S.

88. The method of claim 87, wherein R⁵ is O, R² is H or substituted or unsubstituted C₂₋₈ alkynyl, R³ is H, R⁸ and R^{8′} are OH, Y is H, R is H, Z is H, X is OH, NH₂ or NHOH, W is O, R¹ and R^{1A} are H, and/or R⁴ is OH or O—P(O)R⁶R⁷.

89. A method for treating or preventing a Coronaviridae, Flaviviridae, Picornaviridae, Bunyaviridae, or Togaviridae infection, comprising administering a treatment or preventative amount of a compound of Formula (D) or (D1) to a patient in need of treatment or prevention thereof: 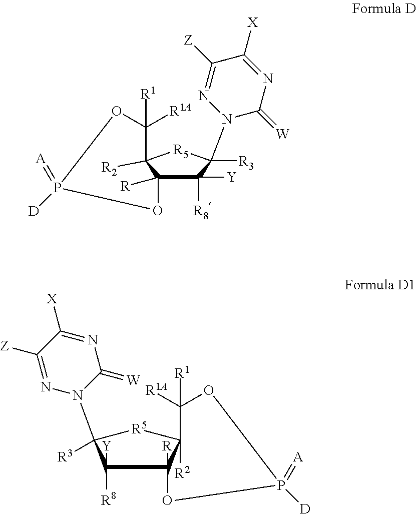
or a pharmaceutically acceptable salt or prodrug thereof, wherein R, R¹, R^{1A}, R², R³, R⁵, R^{8′} and Y are as defined in Formula A, and A and D are as defined in Formula C.

90. The method of claim 89, wherein R⁵ is O, R² is H or substituted or unsubstituted C₂₋₈ alkynyl, R³ is H, R^{8′} is OH, Y is H, R is H, Z is H, X is OH, NH₂ or NHOH, W is O, R¹ and R^{1A} are H, and/or R⁴ is OH or O—P(O)R⁶R⁷.

91. A method for treating or preventing a Coronaviridae, Flaviviridae, Picornaviridae, Bunyaviridae, or Togaviridae infection, comprising administering a treatment or preventative amount of a compound of Formula (E) or (E1) to a patient in need of treatment or prevention thereof: 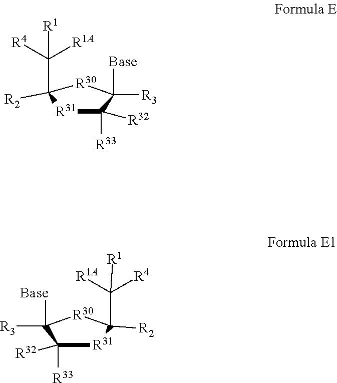
or a pharmaceutically acceptable salt or prodrug thereof, wherein:
Base, R¹, R^{1A}, R², R³, and R⁴ are as defined in Formula A,
R³⁰ is O or CH₂,
R³¹ is O or S,
R³¹ is O when R³⁰ is S, and
R³² and R³³ are independently H, F, C₁-C₃ alkyl, C₂-C₃ alkene, or C₂-C₃ alkyne.

92. The method of claim 91, wherein R³⁰ is O, R³¹ is O, R³² and R³³ are, independently, H or F, R³ is H, R² is N₃ or substituted or unsubstituted C₂₋₈ alkynyl, R¹ and R^{1A} are H, R⁴ is OH or O—P(O)R⁶R⁷, and/or X¹ is N.

93. A method for treating or preventing a Coronaviridae, Flaviviridae, Picornaviridae, Bunyaviridae, or Togaviridae infection, comprising administering a treatment or preventative amount of a compound of Formula (F) or (F1) to a patient in need of treatment or prevention thereof: 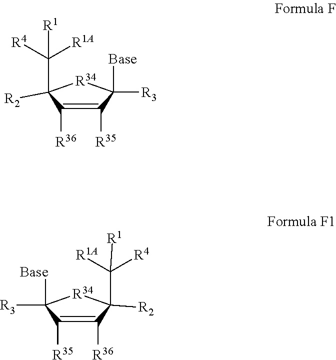
or a pharmaceutically acceptable salt or prodrug thereof, wherein:
Base, R¹, R^{1A}, R², R³, and R⁴ are as defined in Formula A,
R³⁴ is O or CH₂, and
R³⁵ and R³⁶ are independently H, F or CH₃.

94. The method of claim 93, wherein R³⁵ and R³⁶ are H, R³⁴ is CH₂, R⁴ is OH or O—P(O)R⁶R⁷, R³ is H, R² is H or substituted or unsubstituted C₂₋₈ alkynyl, and/or R¹ and R^{1A} are H.

95. The method of any of claims 83-94, wherein the compounds can be present in the β-D or β-L configuration.

96. The method of any of claims 83-94, wherein the virus is a Coronavirus.

97. The method of claim 96, wherein the Coronavirus is SARS-CoV2, MERS, SARS, or OC-43.

98. The method of claim 97, wherein the Coronavirus is SARS-CoV2.

99. The method of any of claims 83-94, wherein the compound is co-administered with one or more additional active compounds selected from the group consisting of fusion inhibitors, entry inhibitors, protease inhibitors, polymerase inhibitors, antiviral nucleosides, viral entry inhibitors, viral maturation inhibitors, JAK inhibitors, angiotensin-converting enzyme 2 (ACE2) inhibitors, SARS-CoV-specific human monoclonal antibodies, including CR3022, and agents of distinct or unknown mechanism.

100. The method of claim 99, wherein the compound is administered with remdesivir, N-hydroxy cytidine, or a pharmaceutically-acceptable salt or prodrug thereof.

101. The method of claim 99, wherein the additional active compound is a JAK inhibitor, and the JAK inhibitor is Jakafi, Tofacitinib, or Baricitinib, or a pharmaceutically-acceptable salt or prodrug thereof.

102. The method of claim 99, wherein the one or more additional active agents comprise an anticoagulant or a platelet aggregation inhibitor.

103. The method of claim 99, wherein the one or more additional active agents comprise an ACE-2 inhibitor, a CYP-450 inhibitor, or NOX inhibitor.

104. The use of a compound of any of claims 83-94 in the preparation of a medicament for use in treating or preventing a Coronaviridae, Flaviviridae, Picornaviridae, Bunyaviridae, or Togaviridae infection.

105. The use of claim 104, wherein the infection is a Coronaviridae infection.

106. The use of claim 105, wherein the Coronavirus is SARS-CoV2, MERS, SARS, or OC-43.

107. The use of claim 106, wherein the Coronavirus is SARS-CoV2.

108. The use of claim 104, wherein the medicament further comprises one or more additional active compounds selected from the group consisting of fusion inhibitors, entry inhibitors, protease inhibitors, polymerase inhibitors, antiviral nucleosides, viral entry inhibitors, viral maturation inhibitors, JAK inhibitors, angiotensin-converting enzyme 2 (ACE2) inhibitors, SARS-CoV-specific human monoclonal antibodies, including CR3022, and agents of distinct or unknown mechanism.

109. The use of claim 108, wherein the medicament further comprises remdesivir, N-hydroxy cytidine, or a pharmaceutically-acceptable salt or prodrug thereof.

110. The use of claim 108, wherein the medicament further comprises a JAK inhibitor, and the JAK inhibitor is Jakafi, Tofacitinib, or Baricitinib, or a pharmaceutically-acceptable salt or prodrug thereof.

111. The use of claim 108, wherein the medicament further comprises an anticoagulant or a platelet aggregation inhibitor.

112. The use of claim 108, wherein the medicament further comprises an ACE-2 inhibitor, a CYP-450 inhibitor, or a NOX inhibitor.

113. The use of claim 108, wherein the medicament is a transdermal composition or a nanoparticulate composition.

114. The method of any of claims 1-72 or 83-103, wherein the compound is administered in combination with an NS5A inhibitor.

115. The method of claim 114, wherein the NS5A inhibitor is daclastavir.

116. The use of any of claims 73-82 or 104-113, wherein the compound is administered in combination with an NS5A inhibitor.

117. The use of claim 116, wherein the NS5A inhibitor is daclastavir.

118. A method for treating or preventing a Coronaviridae, Flaviviridae, Picornaviridae, Bunyaviridae, or Togaviridae infection, comprising administering a treatment or preventative amount of a compound of Formula (A) or Formula (A1) to a patient in need of treatment or prevention thereof: 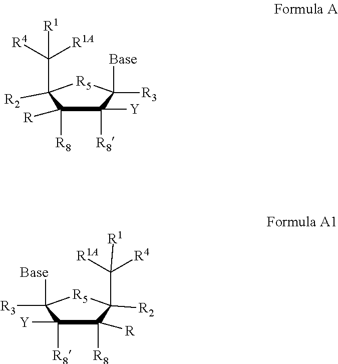
or a pharmaceutically acceptable salt or prodrug thereof, wherein:
Y and R are, independently, selected from the group consisting of H, OH, halo, an optionally substituted O-linked amino acid, substituted or unsubstituted C₁₋₆ alkyl, C₁₋₆ haloalkyl, C₁₋₆ alkoxy, substituted or unsubstituted C₂₋₆ alkenyl, substituted or unsubstituted C₂₋₆ alkynyl, substituted or unsubstituted C₃₋₆ cycloalkyl, cyano, cyanoalkyl, azido, azidoalkyl, OR′, SR′, wherein each R′ is independently a —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, C₁₋₆ alkyl, C₁₋₆ haloalkyl, C₁₋₆ alkoxy, C₂₋₆ alkenyl, C₂₋₆ alkynyl, and C₃₋₆ cycloalkyl, wherein the groups can be substituted with one or more substituents selected from the group consisting of halogen, hydroxyl, amino, alkylamino, arylamino, alkoxy, nitro, and cyano,
R¹ is and R^{1A} are, independently, H, CH₃, CH₂F, CHF₂, or CF₃, wherein, when R¹ is Me, the carbon to which it is attached may be wholly or partially R or S or any mixture thereof, or R¹ and R^{1A} can combine to form a C₃₋₇ cycloalkyl ring;
R² is H, CN, N₃, F, CH₂-halogen, CH₂—N₃, O—CH₂—P—(OH)₃, substituted or unsubstituted C₁₋₈ alkyl, substituted or unsubstituted C₂₋₈ alkenyl or substituted or unsubstituted C₂₋₈ alkynyl;
R³ is H, substituted or unsubstituted C₁₋₈ alkyl, substituted or unsubstituted C₂₋₈ alkenyl, substituted or unsubstituted C₂₋₈ alkynyl, or N₃ when R⁵ is O, and
R³ is selected from the group consisting of H, F, N₃, substituted or unsubstituted (C₁₋₈)alkyl, substituted or unsubstituted (C₂₋₈)alkenyl, substituted or unsubstituted (C₂₋₈)alkynyl, O—(C₁₋₈) alkyl and N₃ when R⁵ is CH₂, Se, CHF, CF₂, —C(CH₃)—, —C(cyclopropyl)-, C═CF₂ or C═CH₂,
R⁵ is O, CH₂, Se, CHF, CF₂, —C(CH₃)—, —C(cyclopropyl)-, C═CF₂ or C═CH₂,
R⁸ and R^{8′} are independently selected from the group consisting of H, OH, halo, an optionally substituted O-linked amino acid, substituted or unsubstituted C₁₋₆ alkyl, C₁₋₆ haloalkyl, C₁₋₆ alkoxy, substituted or unsubstituted C₂₋₆ alkenyl, substituted or unsubstituted C₂₋₆ alkynyl, substituted or unsubstituted C₃₋₆ cycloalkyl, cyano, cyanoalkyl, azido, azidoalkyl, OR′, SR′, wherein each R′ is independently a —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, C₁₋₆ alkyl, C₁₋₆ haloalkyl, C₁₋₆ alkoxy, C₂₋₆ alkenyl, C₂₋₆ alkynyl, C₃₋₆ cycloalkyl, wherein the groups can be substituted with one or more substituents selected from the group consisting of halogen, hydroxyl, amino, alkylamino, arylamino, alkoxy, nitro, and cyano,
R⁴ is OH, an optionally substituted O-linked amino acid, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, —O—C(O)O—C₃₋₆ cycloalkyl, OC₁₋₆ alkyl, OC₁₋₆ haloalkyl, OC₁₋₆ alkoxy, OC₂₋₆ alkenyl, OC₂₋₆ alkynyl, OC₃₋₆ cycloalkyl, O—P(O)R⁶R⁷, O—CH₂—P—(OH)₃, O—CH₂—P—(OH)₃, or a mono-, di-, or triphosphate, wherein, when chirality exists at the phosphorous center of R⁴, it may be wholly or partially Rₚ or Sₚ or any mixture thereof,
R⁶ and R⁷ are independently selected from the group consisting of:
(a) OR¹⁵ where R¹⁵ selected from the group consisting of H, 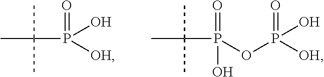
Li, Na, K, substituted or unsubstituted C₁₋₂₀alkyl, substituted or unsubstituted C₃₋₆cycloalkyl, C₁₋₄(alkyl)aryl, benzyl, C₁₋₆ haloalkyl, C₂₋₃(alkyl)OC₁₋₂₀alkyl, aryl, and heteroaryl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
where R¹⁶ is independently H, substituted or unsubstituted C₁₋₂₀ alkyl, the carbon chain derived from a fatty alcohol or C₁₋₂₀ alkyl substituted with a C₁₋₆ alkyl, C₁₋₆ alkoxy, di(C₁₋₆ alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆ alkyl, alkoxy, di(C₁₋₆ alkyl)-amino, fluoro, C₃₋₁₀ cycloalkyl, or cycloalkyl;
(b) the ester of a D- or L-amino acid 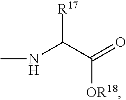
R¹⁷ and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl;
Base is selected from the group consisting of: 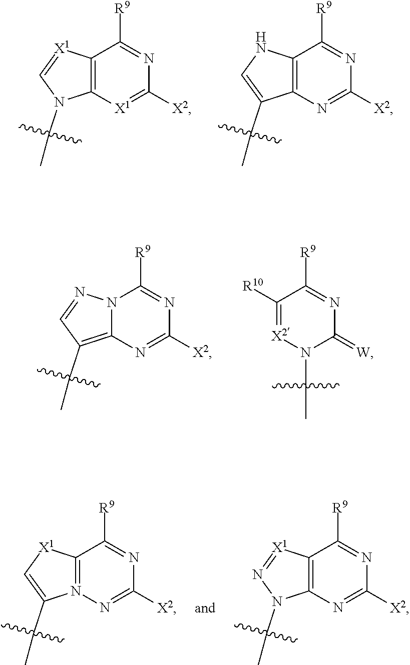
X¹ is CH, C—(C₁₋₆)alkyl, C—(C₂₋₆)alkenyl, C—(C₂₋₆)alkynyl, C—(C₃₋₇)cycloalkyl, C—(C₁₋₆) haloalkyl, C—(C₁₋₆)hydroxyalkyl, C—OR²², C—N(R²²)₂, C-halo, C—CN or N,
X¹ is CH, C—(C₁₋₆)alkyl, C—(C₂₋₆)alkenyl, C—(C₂₋₆)alkynyl, C-halo, C—CN or N
R⁹ and X² are independently H, OH, NH₂, halo (i.e., F, Cl, Br, or I), SH, NHOH, O(C₁₋₁₀)alkyl, O(C₂₋₁₀)alkene, O(C₂₋₁₀)alkyne, O(C₃₋₇)cycloalkyl, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, —O—C(O)O—C₃₋₆ cycloalkyl, S(C₁₋₁₀)alkyl, S(C₂₋₁₀)alkene, S(C₂₋₁₀)alkyne, S(C₃₋₇)cycloalkyl, an optionally unsaturated NH(C₁₋₁₀)alkyl, an optionally unsaturated N((C₁₋₁₀)alkyl)₂, NH(C₃₋₇)cycloalkyl, an optionally unsaturated NH(CO)(C₁₋₂₀)alkyl, an optionally unsaturated NH(CO)O(C₁₋₂₀)alkyl, NHOH, an optionally unsaturated NHO(CO)(C₁₋₂₀)alkyl, or an optionally unsaturated NHO(CO)NH(C₁₋₂₀)alkyl, (C₁₋₃)alkyl,
R^{9′} is OH, NH₂, SH, NHOH, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, or —O—C(O)O—C₃₋₆ cycloalkyl,
R¹⁰ is H or F,
X^{2′} is N or CH, and
W is O or S.
or a compound of Formula (B) or (B1): 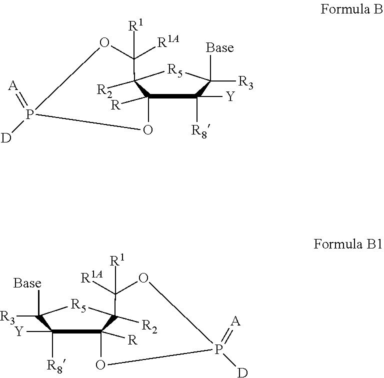
or a pharmaceutically acceptable salt or prodrug thereof, wherein:
Base, Y, R, R¹, R^{1A}, R², R³, R⁵, and R^{8′} are as defined in Formula A,
A is O or S, and
D is selected from the group consisting of:
(a) OR¹⁵ where R¹⁵ is selected from the group consisting of H, substituted or unsubstituted C₁₋₂₀alkyl, substituted or unsubstituted C₃₋₆cycloalkyl, C₁₋₄(alkyl)aryl, benzyl, C₁₋₆ haloalkyl, C₂₋₃(alkyl)OC₁₋₂₀ alkyl, aryl, and heteroaryl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
(b) the ester of a D- or L-amino acid 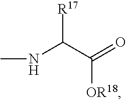
R¹⁷ and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl; and
(c) 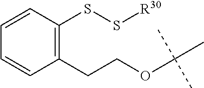
where R³⁰ is selected from the group consisting of substituted or unsubstituted C₁₋₂₀alkyl, substituted or unsubstituted C₃₋₆ cycloalkyl, substituted or unsubstituted (C₂₋₁₀)alkene, substituted or unsubstituted (C₂₋₁₀)alkyne, C₁₋₄(alkyl)aryl, aryl, heteroaryl, and C₁₋₆ haloalkyl,
or a compound of Formula (C) or (C1): 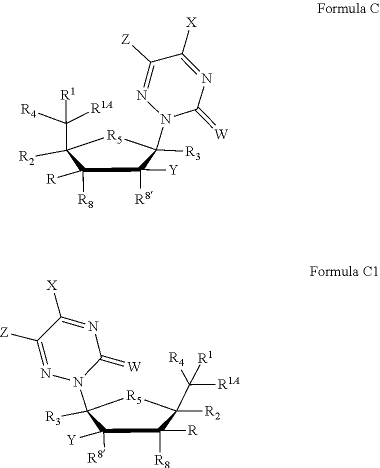
or a pharmaceutically acceptable salt or prodrug thereof, wherein:
R, R¹, R^{1A}, R², R³, R⁵, R⁸, R^{8′} and Y are as defined in Formula A,
X is OH, NH₂, SH, NHOH, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, or —O—C(O)O—C₃₋₆ cycloalkyl,
Z is H or F, and
W is O or S,
or a compound of Formula (D) or (D1): 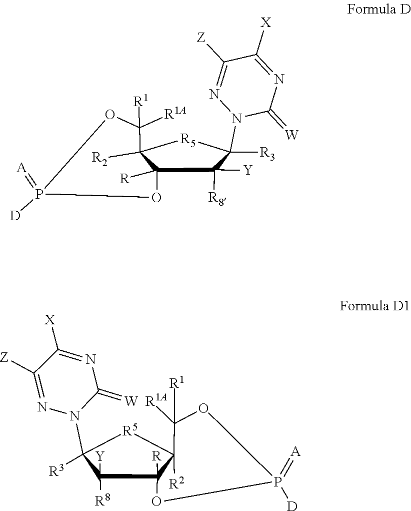
or a pharmaceutically acceptable salt or prodrug thereof, wherein R, R¹, R^{1A}, R², R³, R⁵, R^{8′} and Y are as defined in Formula A, and A and D are as defined in Formula C,
or a compound of Formula E or E1: 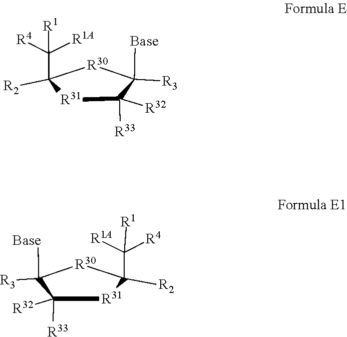
or a pharmaceutically acceptable salt or prodrug thereof, wherein:
Base, R¹, R^{1A}, R², R³, and R⁴ are as defined in Formula A,
R³⁰ is O or CH₂,
R³¹ is O or S,
R³¹ is O when R³⁰ is S, and
R³² and R³³ are independently H, F, C₁-C₃ alkyl, C₂-C₃ alkene, or C₂-C₃ alkyne,
or a compound of Formula (F) or (F1): 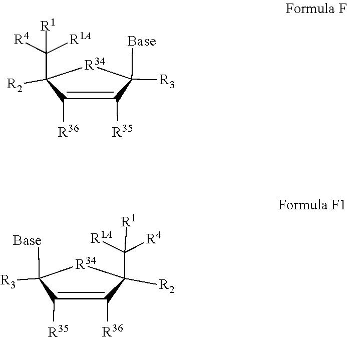
or a pharmaceutically acceptable salt or prodrug thereof, wherein:
Base, R¹, R^{1A}, R², R³, and R⁴ are as defined in Formula A,
R³⁴ is O or CH₂, and
R³⁵ and R³⁶ are independently H, F or CH₃,
or a compound selected from the group consisting of: 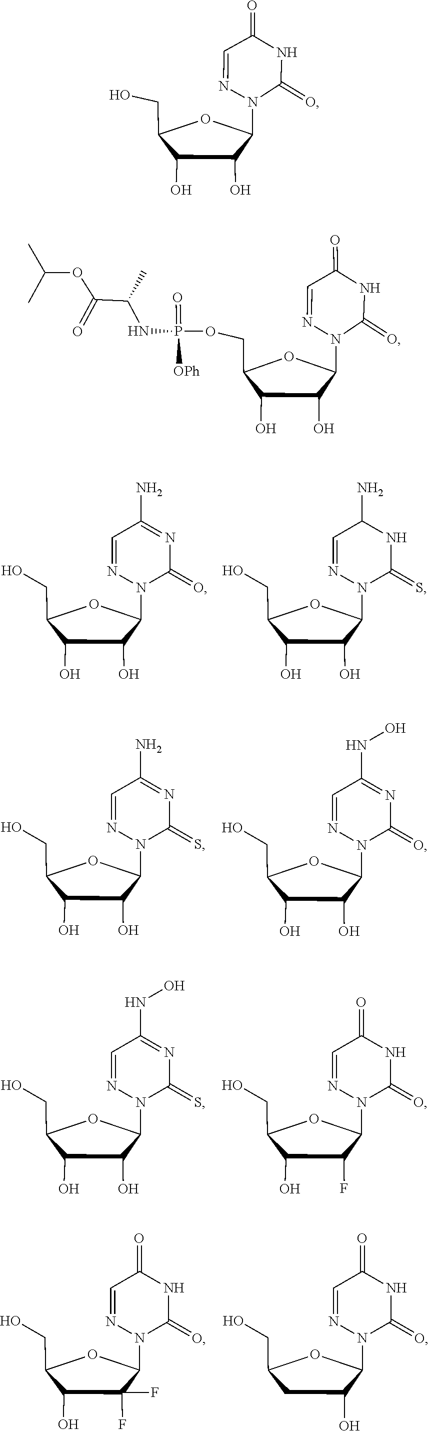 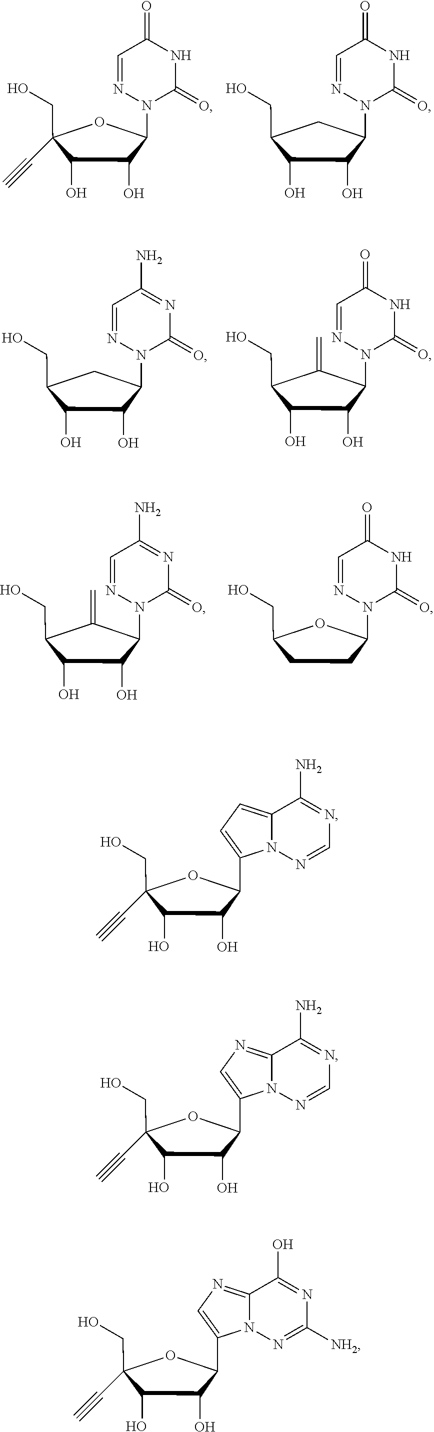 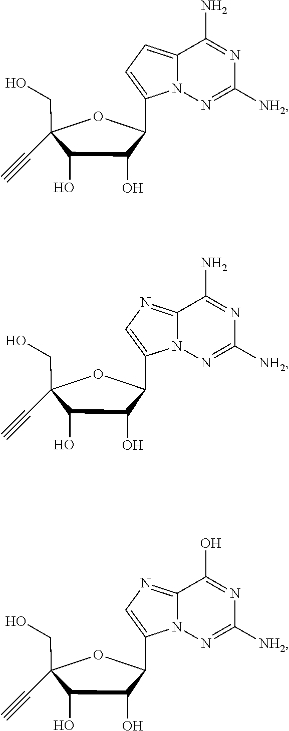 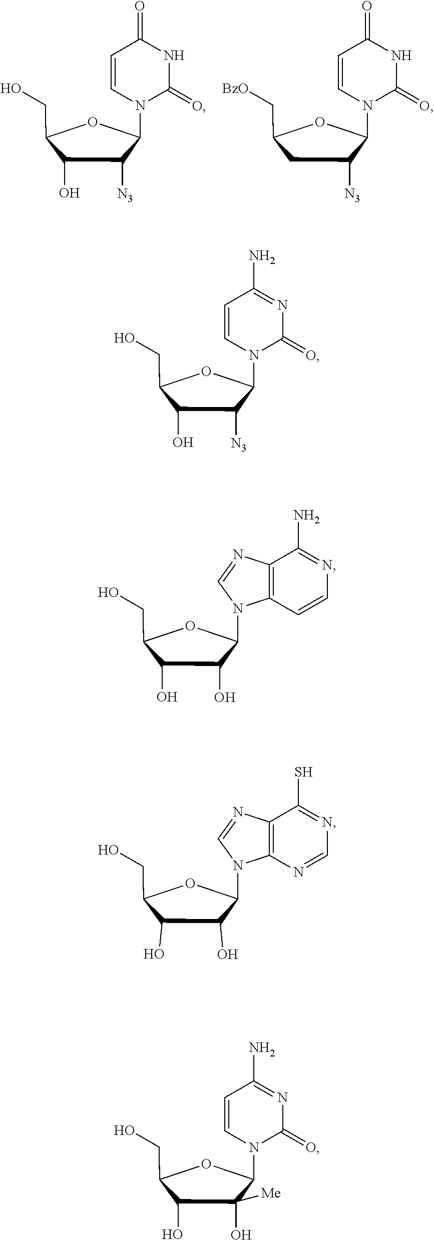 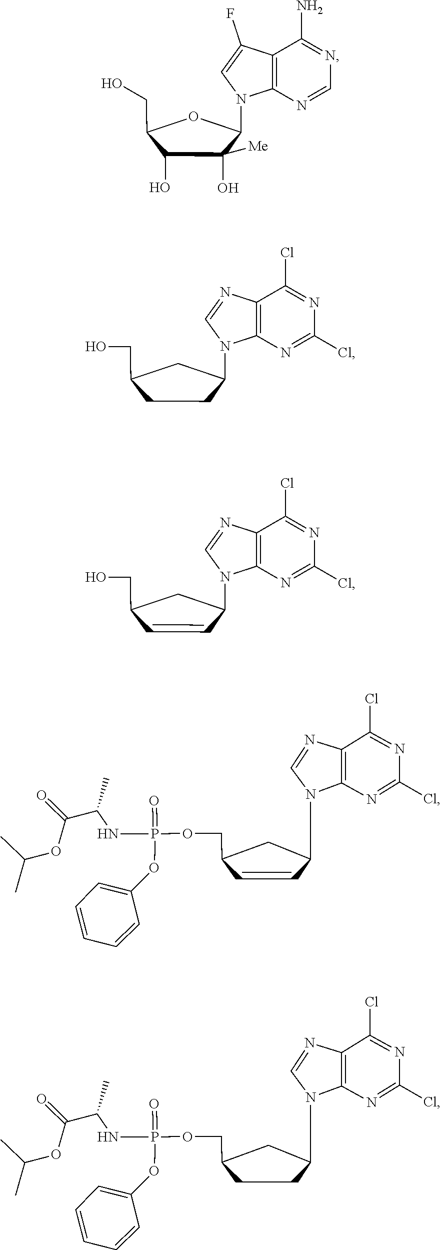 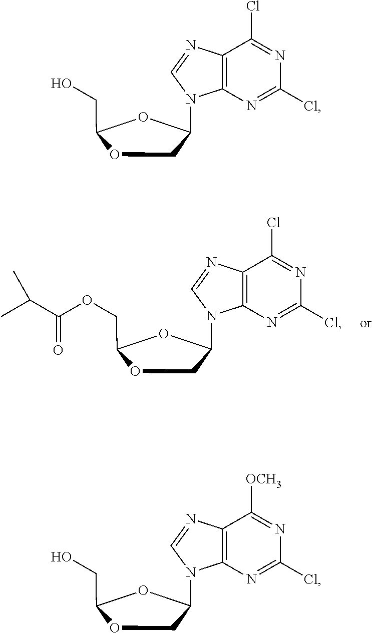
or a pharmaceutically-acceptable salt or prodrug thereof.

119. The method of claim 118, wherein the compounds are of Formula (A) or (A1), and R⁵ is O, R² is H or substituted or unsubstituted C₂₋₈ alkynyl, R³ is H, R is and R^{1A} are H, R⁸ and R^{8′} are OH, R⁴ is OH or O—P(O)R⁶R⁷, R^{9′} is OH, NH₂, or NHOH, and/or X² is NH₂, OH or SH.

120. The method of claim 118, wherein the compounds are of Formula (B) or (B1), and R⁵ is O, R² is H or substituted or unsubstituted C₂₋₈ alkynyl, R³ is H, R′ is OH, Y is H, R¹ and R^{1A} are H, and/or A is O.

121. The method of claim 118, wherein the compounds are of Formula (C) or (C1), and R⁵ is O, R² is H or substituted or unsubstituted C₂₋₈ alkynyl, R³ is H, R⁸ and R^{8′} are OH, Y is H, R is H, Z is H, X is OH, NH₂ or NHOH, W is O, R¹ and R^{1A} are H, and/or R⁴ is OH or O—P(O)R⁶R⁷.

122. The method of claim 118, wherein the compounds are of Formula (D) or (D1), and R⁵ is O, R² is H or substituted or unsubstituted C₂₋₈ alkynyl, R³ is H, R^{8′} is OH, Y is H, R is H, Z is H, X is OH, NH₂ or NHOH, W is O, R¹ and R^{1A} are H, and/or R⁴ is OH or O—P(O)R⁶R⁷.

123. The method of claim 118, wherein the compounds are of Formula (E) or (E1), and R³⁰ is O, R³¹ is O, R³² and R³³ are, independently, H or F, R³ is H, R² is N₃ or substituted or unsubstituted C₂₋₈ alkynyl, R¹ and R^{1A} are H, R⁴ is OH or O—P(O)R⁶R⁷, and/or X¹ is N.

124. The method of claim 118, wherein R³⁵ and R³⁶ are H, R³⁴ is CH₂, R⁴ is OH or O—P(O)R⁶R⁷, R³ is H, R² is H or substituted or unsubstituted C₂₋₈ alkynyl, and/or R¹ and R^{1A} are H.

125. The method of claim 118, wherein the compounds can be present in the β-D or β-L configuration.

126. The method of claim 118, wherein the virus is a Coronavirus.

127. The method of claim 126, wherein the Coronavirus is SARS-CoV2, MERS, SARS, or OC-43.

128. The method of claim 127, wherein the Coronavirus is SARS-CoV2.

129. The method of claim 118, wherein the compound is co-administered with one or more additional active compounds selected from the group consisting of fusion inhibitors, entry inhibitors, protease inhibitors, polymerase inhibitors, antiviral nucleosides, viral entry inhibitors, viral maturation inhibitors, JAK inhibitors, angiotensin-converting enzyme 2 (ACE2) inhibitors, SARS-CoV-specific human monoclonal antibodies, including CR3022, and agents of distinct or unknown mechanism.

130. The method of claim 129, wherein the compound is administered with remdesivir, N-hydroxy cytidine, or a pharmaceutically-acceptable salt or prodrug thereof.

131. The method of claim 129, wherein the additional active compound is a JAK inhibitor.

132. The method of claim 131, wherein the JAK inhibitor is Jakafi, Tofacitinib, or Baricitinib, or a pharmaceutically-acceptable salt or prodrug thereof.

133. The method of claim 129, wherein the one or more additional active agents comprise an anticoagulant or a platelet aggregation inhibitor.

134. The method of claim 129, wherein the one or more additional active agents comprise an ACE-2 inhibitor, a CYP-450 inhibitor, or NOX inhibitor.

134. The method of claim 129, wherein the medicament further comprises an anticoagulant or a platelet aggregation inhibitor.

135. The method of claim 134, wherein the medicament further comprises an ACE-2 inhibitor, a CYP-450 inhibitor, or a NOX inhibitor.

136. The method of claim 129, wherein the compound is administered in combination with an NS5A inhibitor.

137. The method of claim 136, wherein the NS5A inhibitor is daclastavir.

138. The method of claim 118, wherein the compound is administered in a transdermal composition or a nanoparticulate composition.

139. A method for treating or preventing a Coronaviridae, Flaviviridae, Picornaviridae, Bunyaviridae, or Togaviridae infection, comprising administering a treatment or preventative amount of a compound of Formula (A) or Formula (A1) to a patient in need of treatment or prevention thereof: 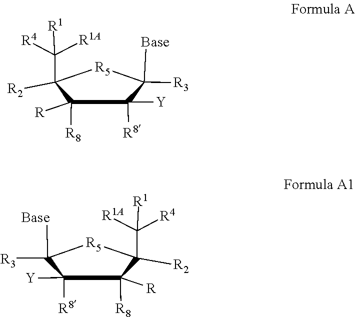
or a pharmaceutically acceptable salt or prodrug thereof, wherein:
Y and R are, independently, selected from the group consisting of H, OH, halo, an optionally substituted O-linked amino acid, substituted or unsubstituted C₁₋₆ alkyl, C₁₋₆ haloalkyl, C₁₋₆ alkoxy, substituted or unsubstituted C₂₋₆ alkenyl, substituted or unsubstituted C₂₋₆ alkynyl, substituted or unsubstituted C₃₋₆ cycloalkyl, cyano, cyanoalkyl, azido, azidoalkyl, OR′, SR′, wherein each R′ is independently a —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, C₁₋₆ alkyl, C₁₋₆ haloalkyl, C₁₋₆ alkoxy, C₂₋₆ alkenyl, C₂₋₆ alkynyl, and C₃₋₆ cycloalkyl, wherein the groups can be substituted with one or more substituents selected from the group consisting of halogen, hydroxyl, amino, alkylamino, arylamino, alkoxy, nitro, and cyano,
R¹ is and R^{1A} are, independently, H, CH₃, CH₂F, CHF₂, or CF₃, wherein, when R¹ is Me, the carbon to which it is attached may be wholly or partially R or S or any mixture thereof, or R¹ and R^{1A} can combine to form a C₃₋₇ cycloalkyl ring;
R² is H, CN, N₃, F, CH₂-halogen, CH₂—N₃, O—CH₂—P—(OH)₃, substituted or unsubstituted C₁₋₈ alkyl, substituted or unsubstituted C₂₋₈ alkenyl or substituted or unsubstituted C₂₋₈ alkynyl;
R³ is H, substituted or unsubstituted C₁₋₈ alkyl, substituted or unsubstituted C₂₋₈ alkenyl, substituted or unsubstituted C₂₋₈ alkynyl, or N₃ when R⁵ is O, and
R³ is selected from the group consisting of H, F, N₃, substituted or unsubstituted (C₁₋₈)alkyl, substituted or unsubstituted (C₂₋₈)alkenyl, substituted or unsubstituted (C₂₋₈)alkynyl, O—(C₁₋₈) alkyl and N₃ when R⁵ is CH₂, Se, CHF, CF₂, —C(CH₃)—, —C(cyclopropyl)-, C═CF₂ or C═CH₂,
R⁵ is O, CH₂, Se, CHF, CF₂, —C(CH₃)—, —C(cyclopropyl)-, C═CF₂ or C═CH₂,
R⁸ and R^{8′} are independently selected from the group consisting of H, OH, halo, an optionally substituted O-linked amino acid, substituted or unsubstituted C₁₋₆ alkyl, C₁₋₆ haloalkyl, C₁₋₆ alkoxy, substituted or unsubstituted C₂₋₆ alkenyl, substituted or unsubstituted C₂₋₆ alkynyl, substituted or unsubstituted C₃₋₆ cycloalkyl, cyano, cyanoalkyl, azido, azidoalkyl, OR′, SR′, wherein each R′ is independently a —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, C₁₋₆ alkyl, C₁₋₆ haloalkyl, C₁₋₆ alkoxy, C₂₋₆ alkenyl, C₂₋₆ alkynyl, C₃₋₆ cycloalkyl, wherein the groups can be substituted with one or more substituents selected from the group consisting of halogen, hydroxyl, amino, alkylamino, arylamino, alkoxy, nitro, and cyano,
R⁴ is OH, an optionally substituted O-linked amino acid, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, —O—C(O)O—C₃₋₆ cycloalkyl, OC₁₋₆ alkyl, OC₁₋₆ haloalkyl, OC₁₋₆ alkoxy, OC₂₋₆ alkenyl, OC₂₋₆ alkynyl, OC₃₋₆ cycloalkyl, O—P(O)R⁶R⁷, O—CH₂—P—(OH)₃, O—CH₂—P—(OH)₃, or a mono-, di-, or triphosphate, wherein, when chirality exists at the phosphorous center of R⁴, it may be wholly or partially Rₚ or Sₚ or any mixture thereof,
R⁶ and R⁷ are independently selected from the group consisting of:
(a) OR¹⁵ where R¹⁵ selected from the group consisting of H, 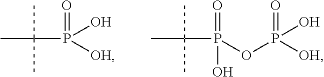
Li, Na, K, substituted or unsubstituted C₁₋₂₀alkyl, substituted or unsubstituted C₃₋₆cycloalkyl, C₁₋₄(alkyl)aryl, benzyl, C₁₋₆ haloalkyl, C₂₋₃(alkyl)OC₁₋₂₀alkyl, aryl, and heteroaryl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
where R¹⁶ is independently H, substituted or unsubstituted C₁₋₂₀ alkyl, the carbon chain derived from a fatty alcohol or C₁₋₂₀ alkyl substituted with a C₁₋₆ alkyl, C₁₋₆ alkoxy, di(C₁₋₆ alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆ alkyl, alkoxy, di(C₁₋₆ alkyl)-amino, fluoro, C₃₋₁₀ cycloalkyl, or cycloalkyl;
(b) the ester of a D- or L-amino acid 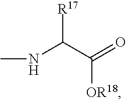
R¹⁷ and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl;
Base is 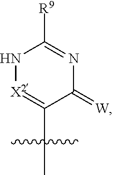
R⁹ is H, OH, NH₂, halo, SH, NHOH, O(C₁₋₁₀)alkyl, O(C₂₋₁₀)alkene, O(C₂₋₁₀)alkyne, O(C₃₋₇)cycloalkyl, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, —O—C(O)O—C₃₋₆ cycloalkyl, S(C₁₋₁₀)alkyl, S(C₂₋₁₀)alkene, S(C₂₋₁₀)alkyne, S(C₃₋₇)cycloalkyl, an optionally unsaturated NH(C₁₋₁₀)alkyl, an optionally unsaturated N((C₁₋₁₀)alkyl)₂, NH(C₃₋₇)cycloalkyl, an optionally unsaturated NH(CO)(C₁₋₂₀)alkyl, an optionally unsaturated NH(CO)O(C₁₋₂₀)alkyl, NHOH, an optionally unsaturated NHO(CO)(C₁₋₂₀)alkyl, or an optionally unsaturated NHO(CO)NH(C₁₋₂₀)alkyl, (C₁₋₃)alkyl,
X^{2′} is N or CH, and
W is O or S.
or a compound of Formula (B) or (B1): 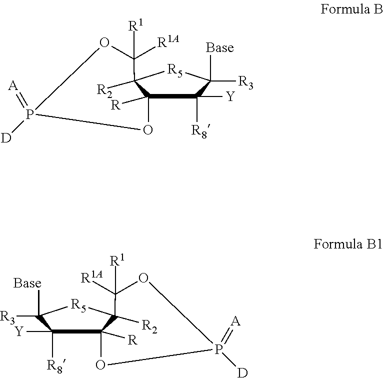
or a pharmaceutically acceptable salt or prodrug thereof, wherein:
Base, Y, R, R¹, R^{1A}, R², R³, R⁵, and R^{8′} are as defined in Formula A,
A is O or S, and
D is selected from the group consisting of:
(a) OR¹⁵ where R¹⁵ is selected from the group consisting of H, substituted or unsubstituted C₁₋₂₀alkyl, substituted or unsubstituted C₃₋₆cycloalkyl, C₁₋₄(alkyl)aryl, benzyl, C₁₋₆ haloalkyl, C₂₋₃(alkyl)OC₁₋₂₀ alkyl, aryl, and heteroaryl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
(b) the ester of a D- or L-amino acid 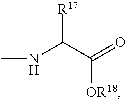
R¹⁷ and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl; and
(c) 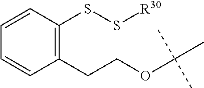
where R³⁰ is selected from the group consisting of substituted or unsubstituted C₁₋₂₀alkyl, substituted or unsubstituted C₃₋₆ cycloalkyl, substituted or unsubstituted (C₂₋₁₀)alkene, substituted or unsubstituted (C₂₋₁₀)alkyne, C₁₋₄(alkyl)aryl, aryl, heteroaryl, and C₁₋₆ haloalkyl,
or a compound of Formula (C) or (C1): 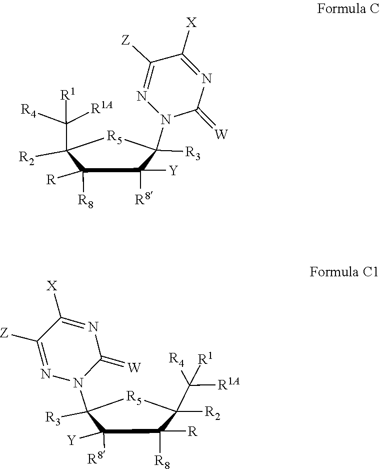
or a pharmaceutically acceptable salt or prodrug thereof, wherein:
R, R¹, R^{1A}, R², R³, R⁵, R⁸, R^{8′} and Y are as defined in Formula A,
X is OH, NH₂, SH, NHOH, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, or —O—C(O)O—C₃₋₆ cycloalkyl,
Z is H or F, and
W is O or S,
or a compound of Formula (D) or (D1): 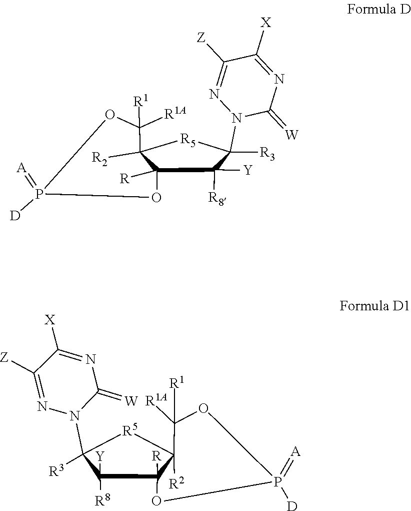
or a pharmaceutically acceptable salt or prodrug thereof, wherein R, R¹, R^{1A}, R², R³, R⁵, R^{8′} and Y are as defined in Formula A, and A and D are as defined in Formula C,
or a compound of Formula E or E1: 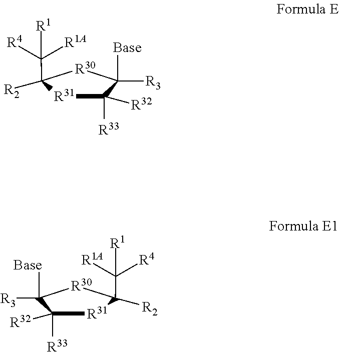
or a pharmaceutically acceptable salt or prodrug thereof, wherein:
Base, R¹, R^{1A}, R², R³, and R⁴ are as defined in Formula A,
R³⁰ is O or CH₂,
R³¹ is O or S,
R³¹ is O when R³ is S, and
R³² and R³³ are independently H, F, C₁-C₃ alkyl, C₂-C₃ alkene, or C₂-C₃ alkyne,
or a compound of Formula (F) or (F1): 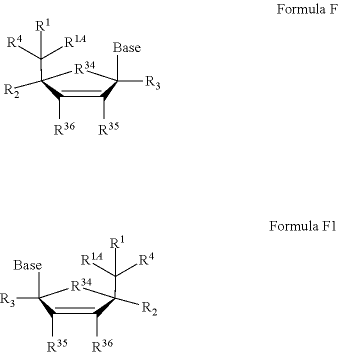
or a pharmaceutically acceptable salt or prodrug thereof, wherein:
Base, R¹, R^{1A}, R², R³, and R⁴ are as defined in Formula A,
R³⁴ is O or CH₂, and
R³⁵ and R³⁶ are independently H, F or CH₃,
or a pharmaceutically-acceptable salt or prodrug thereof.
